SEQUENCE LISTING (42 sequences)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 4 aa, group 3 coronavirus infectious bronchitis virus
  misc_feature (3)..(4): Xaa can be any naturally occurring amino acid
KKXX

SEQ ID NO: 3 - protein, 42 aa, group 3 coronavirus infectious bronchitis virus
GCCGCCCGCFGIPLMSKCGKKSSYYTFDNDVVIEQYRPKKSV

SEQ ID NO: 4 - protein, 5 aa, group 1 coronaviruses
  misc_feature (2)..(2): Xaa can be any naturally occurring amino acid
  misc_feature (4)..(5): Xaa can be any naturally occurring amino acid
KXHXX

SEQ ID NO: 5 - protein, 42 aa, Artificial Sequence
  Synthetic polypeptide
GCCGCCCGCFGIPLMSKCGKKSSYYTFDNDVVIEQYRPAASV

SEQ ID NO: 6 - protein, 42 aa, Artificial Sequence
  Synthetic polypeptide
GCCGCCCGCFGIPLMSKCGKKSSAATFDNDVVIEQYRPAASV

SEQ ID NO: 7 - protein, 44 aa, Artificial Sequence
  Synthetic polypeptide
GCCGCCCGCFGIIPLMSKCGKKSSAATTFDNDVVTEQYRPAASV

SEQ ID NO: 8 - protein, 50 aa, Artificial Sequence
  Synthetic polypeptide
GCCGCCCGCFGIIPLMSKCGKKSSAATTFDNDVVTEQYRPAASVHHHHHH

SEQ ID NO: 9 - DNA, 38355 bp, Artificial Sequence
  Synthetic nucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcggatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctttcaaaggaggtacccaccatgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgacatcagcggaatcaatgccagcgtcgtgaacatccagaaagagatcgaccggctgaacgaggtggccaagaatctgaacgagagcctgatcgaccttcaagaactggggaagtacaaaacttatattaaggcgcctgcgtatgtggcgttagccatagcttttgccactattatcttcatcttaatacttggatggttgtttttcatgactggttgttgtggttgttgttgtggatgctttggcattattcctttaatgagtaagtgtggtaagaaatcttctgctgccacgacttttgataatgatgtggtaactgaacaatacagacctgccgcgtctgtctagcactgagatatcctagacccagctttcttgtacaaagtggttgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatccgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctggatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatccgcatgcgatatcgagctctcccgggaattcggatctgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaaggatccgaattcccgggagagctcgatatcgcatgcggatttaaattaattaa

SEQ ID NO: 10 - protein, 9 aa, SARS-CoV-2
YIKWPWYIW

SEQ ID NO: 11 - protein, 6 aa, Artificial Sequence
  Synthetic polypeptide
  misc_feature (2)..(2): Xaa can be any naturally occurring amino acid
  misc_feature (4)..(5): Xaa can be any naturally occurring amino acid
WXWXXW

SEQ ID NO: 12 - protein, 6 aa, Artificial Sequence
  Synthetic polypeptide
  misc_feature (2)..(2): Xaa can be any naturally occurring amino acid
  misc_feature (4)..(5): Xaa can be any naturally occurring amino acid
AXAXXA

SEQ ID NO: 13 - protein, 6 aa, Artificial Sequence
  Synthetic polypeptide
WPWYIW

SEQ ID NO: 14 - protein, 6 aa, Artificial Sequence
  Synthetic polypeptide
APAYIA

SEQ ID NO: 15 - protein, 6 aa, Artificial Sequence
  Synthetic polypeptide
APAYVA

SEQ ID NO: 16 - protein, 76 aa, Artificial Sequence
  Synthetic polypeptide
KTYIKAPAYVALAIAFATIIFILILGWLFFMTGCCGCCCGCFGIIPLMSKCGKKSSAATTFDNDVVTEQYRPAASV

SEQ ID NO: 17 - protein, 1281 aa, Artificial sequence
  Synthetic polypeptide
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYKTYIKAPAYVALAIAFLTIIFILILGWVFFMTGCCGCCCGCFGIIPLMSKCSKKSSAATTFDNDVVTEQYRPAASV

SEQ ID NO: 18 - protein, 1281 aa, Artificial Sequence
  Synthetic polypeptide
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKLYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYKTYIKAPAYVALAIAFATIIFILILGWLFFMTGCCGCCCGCFGIIPLMSKCGKKSSAATTFDNDVVTEQYRPAASV

SEQ ID NO: 19 - DNA, 20 bp, Artificial Sequence
  SwTMF2aEnv4b_1_F primer
ggtacccaccatgttcgtgt

SEQ ID NO: 20 - DNA, 20 bp, Artificial Sequence
  SwTMF2aEnv4b_1_R primer
atctgaacaccttgtcgggg

SEQ ID NO: 21 - DNA, 20 bp, Artificial Sequence
  SwTMF2aEnv4b_2_F primer
cgtggtcatcaaagtgtgcg

SEQ ID NO: 22 - DNA, 20 bp, Artificial Sequence
  SwTMF2aEnv4b_2_R primer
tccatcaggaaaggctggga

SEQ ID NO: 23 - DNA, 20 bp, Artificial Sequence
  SwTMF2aEnv4b_3_F primer
tggatcctctgagcgagaca

SEQ ID NO: 24 - DNA, 19 bp, Artificial Sequence
  SwTMF2aEnv4b_3_R primer
cgaaggggcacagattggt

SEQ ID NO: 25 - DNA, 20 bp, Artificial Sequence
  SwTMF2aEnv4b_4_F primer
ggctgttccggaagtccaat

SEQ ID NO: 26 - DNA, 20 bp, Artificial Sequence
  SwTMF2aEnv4b_4_R primer
tgatagcccacgccatttgt

SEQ ID NO: 27 - DNA, 20 bp, Artificial Sequence
  SwTMF2aEnv4b_5_F primer
tcagctgacacctacatggc

SEQ ID NO: 28 - DNA, 20 bp, Artificial Sequence
  SwTMF2aEnv4b_5_R primer
atggggatgtcgcactcgta

SEQ ID NO: 29 - DNA, 20 bp, Artificial Sequence
  SwTMF2aEnv4b_6_F primer
acttcggcggcttcaatttc

SEQ ID NO: 30 - DNA, 20 bp, Artificial Sequence
  SwTMF2aEnv4b_6_R primer
gcaatgtcgcccagacaatc

SEQ ID NO: 31 - DNA, 20 bp, Artificial Sequence
  SwTMF2aEnv4b_7_F primer
actgaacaccctggtcaagc

SEQ ID NO: 32 - DNA, 20 bp, Artificial Sequence
  SwTMF2aEnv4b_7_R primer
tcagtctgtcgatctgcacc

SEQ ID NO: 33 - DNA, 20 bp, Artificial Sequence
  SwTMF2aEnv4b_8_F primer
accttcgtgtctggcaactg

SEQ ID NO: 34 - DNA, 37 bp, Artificial Sequence
  SIBV_FOR primer
aggaactggataagtactttaagaaccacacaagccc

SEQ ID NO: 35 - DNA, 20 bp, Artificial Sequence
  SwTMF2aEnv4b_8_R primer
gccggtcgatctctttctgg

SEQ ID NO: 36 - DNA, 39 bp, Artificial Sequence
  SIBV_REV primer
gaaagctgggtctaggatatctcagtgctagacagacgc

SEQ ID NO: 37 - protein, 17 aa, Artificial Sequence
  V5 tag
GKPIPNPLLGLDSTRTG

SEQ ID NO: 38 - DNA, 966 bp, Artificial Sequence
  AmpR promoter and gene
cgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 39 - DNA, 38355 bp, Artificial Sequence
  Synthetic nucleotide
ttaattaatttaaatccgcatgcgatatcgagctctcccgggaattcggatccttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcagatccgaattcccgggagagctcgatatcgcatgcggatttaaattaattaacatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagtgacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaaatgtttttttgttattttattttgtgtttatgcagaaacccgcagacatgtttgagagaaaaatggtgtctttttctgtggtggttccggagcttacctgcctttatctgcatgagcatgactacgatgtgctttcttttttgcgcgaggctttgcctgattttttgagcagcaccttgcattttatatcgccgcccatgcaacaaagcttacatcggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagatatattgagaaggtggcgagatgagaattatttgggcatggttgaaggtgctggaatgtttatagaggagattcaccctgaagggtttagcctttacgtccacttggacgtgagggccgtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgatctaagcggcgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgattcgaaattttctgcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgtttagtgtaatggttagtgttacaggtttagttttgtctccgtttaagtaaacttgactgacaatgttacttttggcagttttaccgtgagattttggataagctgataggttaggcataaatccaacagcgtttgtataggctgtgccttcagtaagatctccatttctaaagttccaatattctgggtccaggaaggaattgtttagtagcactccattttcgtcaaatcttataataagatgagcactttgaactgttccagatattggagccaaactgcctttaacagccaaaactgaaactgtagcaagtatttgactgccacattttgttaagaccaaagtgagtttagcatctttctctgcatttagtctacagttaggagatggagctggtgtggtccacaaagttagcttatcattatttttgtttcctactgtaatggcacctgtgctgtcaaaactaaggccagttcctagtttaggaaccatagccttgtttgaatcaaattctaggccatggccaatttttgttttgaggggatttgtgtttggtgcattaggtgaaccaaattcaagcccatctcctgcattaatggctatggctgtagcgtcaaacatcaaccccttggcagtgcttaggttaacctcaagctttttggaattgtttgaagctgtaaacaagtaaaggcctttgttgtagttaatatccaagttgtgggctgagtttataaaaagagggccctgtcctagtcttagatttagttggttttgagcatcaaacggataactaacatcaagtataaggcgtctgttttgagaatcaatccttagtcctcctgctacattaagttgcatattgccttgtgaatcaaaacccaaggctccagtaactttagtttgcaaggaagtattattaatagtcacacctggaccagttgctacggtcaaagtgtttaggtcgtctgttacatgcaaaggagccccgtactttagtcctagttttccattttgtgtataaatgggctctttcaagtcaatgcccaagctaccagtggcagtagttagagggggtgaggcagtgatagtaagggtactgctatcggtggtggtgagggggcctgatgtttgcagggctagctttccttctgacactgtgaggggtccttgggtggcaatgctaagtttggagtcgtgcacggttagcggggcctgtgattgcatggtgagtgtgttgcccgcgaccattagaggtgcggcggcagccacagttagggcttctgaggtaactgtgaggggtgcagatatttccaggtttatgtttgacttggtttttttgagaggtgggctcacagtggttacattttgggaggtaaggttgccggcctcgtccagagagaggccgttgcccattttgagcgcaagcatgccattggaggtaactagaggttcggataggcgcaaagagagtaccccagggggactctcttgaaacccattgggggatacaaagggaggagtaagaaaaggcacagttggaggaccggtttccgtgtcatatggatacacggggttgaaggtatcttcagacggtcttgcgcgcttcatctgcaacaacatgaagatagtgggtgcggatggacaggaacaggaggaaactgacattccatttagattgtggagaaagtttgcagccaggaggaagctgcaataccagagctgggaggagggcaaggaggtgctgctgaataaactggacagaaatttgctaactgattttaagtaagtgatgctttattatttttttttattagttaaagggaataagatctttgagaccgcacagggtcttaataagggtgcagagatcctcaggtccttgacaaggtgagtgaatgcagccttcggtttctaccgagtgctgagttatggtaatgggcttttctcccaccatgaccaccaatttctgacgcttggttggcaacttgtagctaaggcggtgtccggtggtattactgtcgtaggtgactttggcctgctttaccagacaaaagataccccttttgcactggtgcaagttaaccatgtcttggagctcttgattcatgcgctgttgctcggccgctgccctgcgtctttctagcaggcgctgctctgtaataattccgtccatttctagagaaacctgaattagaatagcccgtagagttgcttgaattgttcataaaccccacagtagctgcgcctttggcctaataccctaagggttttctaagctcacctcctgttctggtaaacagagttattgaggtctgtccggaaaaagtctggtttacggtcaggcggtaggtgtggtgcagcggccggtgacgcactcgtacgttcccggcaggtaaggagggtggtgttttttctgatggagtagctgagctcggagaggttctctcgtagactcactccgtctgggttgaaactgttgtaaatcacagagggagagatgttaaaagtaccaggtaaggttcgccttggtttgcttgggcgggtgaagacggtggcgtttacaggatggcgataggagccccagtatattttaatttctgtatttattatactcagcacagagatggcaacaaagatcttgatgtaatccagggttaggacagttgcaaatcacagtgagaacacagggtcccctgtcccgctcaactagcagggggcgctgggtaaactcccgaatcaggctacgggcaagctctccctgggcggtaagccggacgccgtgcgccgggccctcgatatgatcctcgggcaattcaaagtagcaaaactcaccggagtcgcgggcaaagcacttgtggcggcgacagtggaccaggtgtttcaggcgcagttgctctgcctctccacttaacattcagtcgtagccgtccgccgagtcctttaccgcgtcaaagttaggaataaattgatccggatagtggccgggaggtcccgagaaggggttaaagtagaccgatggcacaaactcctcaataaattgcagagttccaatgcctccagagcgcggctcagaggacgaggtctgcagagttaggattgcctgacgaggcgtgaatgaagagcggccggcgccgccgatctgaaatgtcccgtccggacggagaccaagcgaggagctcaccgactcgtcgttgagctgaatacctcgccctctgattgtcaggtgagttataccctgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttccaggagaattcggtttcggtgggcgcgtattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacaacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcattaggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcggcgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtgggggtaaataatcacccgagagtgtacaaataaaagcatttgcctttattgaaagtgtctctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaataaggttcatggtggggttgtgatccatgggagtttggggccagttggcaaaggcgtggagaaacatgcagcagaatagtccacaggcggccgagttgggcccctgtacgctttgggtggacttttccagcgttatacagcggtcgggggaagaagcaatggcgctacggcgcaggagtgactcgtactcaaactggtaaacctgcttgagtcgctggtcagaaaagccaaagggctcaaagaggtagcatgtttttgagtgcgggttccaggcaaaggccatccagtgtacgcccccagtctcgcgaccggccgtattgactatggcgcaggcgagcttgtgtggagaaacaaagcctggaaagcgcttgtcataggtgcccaaaaaatatggcccacaaccaagatctttgacaatggctttcagttcctgctcactggagcccatggcggcagctgttgttgatgttgcttgcttctttatgttgtggcgttgccggccgagaagggcgtgcgcaggtacacggtttcgatgacgccgcggtgcggctggtgcacacggaccacgtcaaagacttcaaacaaaacataaagaagggtgggctcgtccatgggatccacctcaaaagtcatgtctagcgcgtgggcggagttggcgtagagaaggttttggcccaggtctgtgagtgcgcccatggacataaagttactggagaatgggatgcgccaaagggtgcgatcgcaaagaaactttttctgggtaatgctgtcaactgcggtcttgcctataagcggataggggaagttagcagggtaggcctgtccttcgcgcatggtgggggcaaggtagccaacaaatccagagttgttgtgttggtgtaggatgcccacctgttggtagtccttgtatttagtatcatccaccacctgacggctcatgggctggaagtttctaaagaaggagtacatgcggtccttgtagctctctgggatatagaagccctggtagccaatgttatagttagctagcatttgtaccaggaaccagtctttggtcatgttacactgggcaacgttgtaaccctccccgtcaactgagcgcttaatttcaaactcgttgggggtaagcaggcggtcattgccaggccagctgacagaagagtcaaaggtaatggccaccttcttaaaggtgtggttgaggtaaaaggttccatctaggtagggtatagagccagagtaggtgtaataagggtcgtagcccgagcccagtgatggggtttccttagtcttaaggcgcgtgaaggcccagccgcggaaagccgcccagttgcgggaggggatggatatgggcacgttggtagcgttggcgggtatagggtagagcatgttggcggcggagagatagtcgttaaaggactggtcgttggtgtcgtttctaagcatggcctcaagcgtggaggcggtgttgtgggccatggggaagaaggtggcgtaaaggcaaatgctatcaaacttaatgctggctccgtcaacccttaggtcatttcctagggagctctgcagaaccatgttaacatccttcctgaagttccactcgtaggtgtatgagcccggcaggagaaggaggtttttaatggcaaagaacttctgaggcacctggatgtggaagggcacatagcgaccattgcccagcaacattgagcggtagcgcaggccagcattgcggtggtggttaaatgggttgacgttgtccatatagtcaagggaccagcgtgctccaaggttaatgtagcagtccactagcccgggagccaccactcgcttgttcatgtagtcgtaggtgtttgggttatcagaaatttttacgttggaaggactgtactttagcttgtcgggcaaatacagcgctatgttggagtacaggaaatttctccacaggttggcatttagattgatttccatggcaaaattatttccaactcttatttcatttttatctgaaaattctgtagcatctttttcccatccattttcctgacctgttttaggttttaccttggtaagagtctctgtattaatcacacctcccagtggaaagcagtaatttggaagttcatcttcagttccatgattttcaataattctaacatctggatcatagctgtcaacagcctgattccacatagaaaagtacctggttctatcaccaatggaatcaagcaaaagctggtatgaaagctctgtgtttctgtcttgcaaatctacaacagcattcaactgcgatgcttggcccgccagaacacccatattacccgtgctgttgtaatacattagaccaataaaattgtccctaaaagcaatgtaattaggcctgttgggcatagattgttggcccattagttctcgtgagttaccttccttaatagtgggcatgtaagaaatatgagtgtctggggtttctatatctacatcttcactgtacaataccactttaggagtcaagttatcaccattgcctgcggctgcctcagtagttgagaaaaattgcatttccacttgactttctagctttccattttgttgctttacaagaatgccttgccctccattttcatttgtgggttttgcatatgaaccgtaacatggtttcattggggtagtctttttaaggactctcccagctgcatgattaatttctgtttcgtaccactgagattctcctatttgaggttcaggttgaaatgttttatcggcatatttaggtgtttgaccttcgacacctatttgaataccctcctttgtaatatttataccagaataaggcgcctgcccaaatacgtgagttttttgctgctcagcttgctcgtctacttcgtcttcgttgtcatcgtcctcttcttctaggtttatttcaagagcagtagcagcttcatcccattcgcaaggatttggggcacccttgggagccagggcgttgtaggcagtgccagagtagggcttaaaagtagggcccctgtccagcacgccgcggatgtcaaagtacgtggaagccatgtccagcacacggttatcacccacagctagggtgaaccgcgccttgtacgagtacgcagtatcctcacggtccacagggatgaaccgcagcgtcaaacgctgggaccggtctgtggtcacgtcgtgcgtaggcgccaccgtggggtttctaaacttgttattcaggctgaagtacgtctcggtggcgcgggcaaactgcaccagcccggggctcaggtactccgaggcgtcctggcccgagatgtgcatgtaagaccactgcggcatcatcgaaggggtagccatcttggaaagcgggcgcgcggcggctcagcagctcctctggcggcgacatggacgcatacatgacacacatacgacacgttagctatccagaagcatcgtcggcgcttcagggattgcacccccagacccacgatgctgttcagtgtgctttgccagttgccactggctacgggccgcaacgatcgcggaccgctggcggcgcggcgcagggacgcgcggctaggacgggttacaacaacggcggtcgggcctggcagcacaggtttctgctgggtgtcggcggggggaggcaggtccagcgttacgggtgtgtgctggcccagcactccggtagccatgggcgcgatgggacgggtggtgggcaggccttgctttagtgcctcctcgtacgagggaggctcgtctatttgcgtcaccagagtttcttccctgtcggggcgcggacgcttttcgccacgcccctctggagacactgtctccacggccggtggaggctcctctacgggagggcggggatcaagcttactgttaatcttattttgcactgcctggttggccaggtccaccaccccgctaatgccagaggccaggccatctaccaccttttgttggaaattttgctctttcaacttatccctcagcatctggcctgtgctgctgttccaggccttgctgccatagttcttaacggtggaaccgaaatttttaatgccgctccacagcgagccccagctgaaggcgccaccgctcatattgctggtgccgatatcttgccagtttcccatgaacgggcgcgagccgtgtcgcggggccagagacgcaaagttgatgtcttccattctacaaaatagttacaggaccaagcgagcgtgagagtccagactttttattttgatttttccacatgcaacttgtttttaatcagtgtctctgcgcctgcaaggccacggatgcaattccgggcacggcgccaatcgccgcggcgatcagtggaataaggaggggcaggataccgccgcgcatgcgacggtgcgacgcgcgccgccgccggtggtgcgcacgacgcatgccgcccgtcaggccgtggccggccatgcccctcctacggtgcattcttcctcggaatcccggcaccgggaaacggaggcggcaggtgagggccatatctgcaagaaccacaaagaccggcttttaaacgatgctggggtggtagcgcgctgttggcagcaccagggtcctgcctccttcgcgagccaccctgcgcacggaaatcggggccagcacgggctggcgacggcgacggcggcggcgggttccagtggtggttcggcgtcgggtagttgctcgtcttctggggcggtaggtgtagccacgatagccgggggtaggcgcaatggaaggatgtagggcatattcgggcagtagcgcgctggcggcgccgtacttcctcgaacggcgcgggcgccggggggctgaaacgcgaaacatccacgggtccgtttgcacctccgtagaggtcttggacgcggccgcagcgaccgcctgcaccgcggcatccgccaccgctgaggcaaccggggacgtttgtgtctccatgccctctgtggcggtggcaatactggtgctactggtagtgggtatctgaacgtccacggtctgcacgcccagtcccggcgccacctgcttgattggccgcacgcggacctcgggctccagcccaggttccacggtcattttttccaagacatcttccagtcgctggcgcttgggtaccatcagctgcacggtgggtgccaagtcaccagactcgcgctttaggccgcgcttttcttcggacggtgcaagcgcgggcagcacctgctgcagtgttacgggctttaggctaggtgttgggttgccctcgtccagcggcaacgccagcatgtccttatgccgctttccgtaggcaaactccccgaggcgctcgttggcctgctcaagcaggtcctcgtcgccgtacacctcatcatacacgcgcttgtaggtgcgggtggagcgctcaccgggcgtaaagactacggtggtgccgggtcgcaaaacacgttttacgcgtcgacctttccactgtacccgtcgcctgggcgcggtagcgtgcagcagttccacctcgtcgtcaagttcatcatcatcatctttctttttctttttgacccgctttagctttcggggcttgtaatcctgctcttccttcttcggggggccatagatctccggcgcgatgacctggagcatctcttctttgattttgcgcttggacatagcttcgttgcgcgccgccgccgctggatacatacaacagtacgagtctaagtagttttttcttgcaatctagttgcgcggggggcgggtgcgcacgggcacgcgcaggccgctaaccgagtcgcgcacccaatacacgttgcccctgcgaccctgagtcatagcactaatggccgcggctgctgcggcggccgctcgtcgcctggacctggggggcacagtgacaatacccgcggccagccttcgagcggcccgcatggccgcccgtcggccggtgcgacgtgcgcggttaagcagggccgccgccgcgcgttgggcggcagtgccgggtcggcggcggtggcgacgtgctacgcgcctccgccgtctcttcattttagcatagcgccgggctccgcgcaccacggtctgaatggccgcgtccactgtggacactggtggcggcgtgggcgtgtagttgcgcgcctcctccaccaccgcgtcgatggcgtcatcgacggtggtgcgcccagtgcggccgcgtttgtgcgcgccccagggcgcgcggtagtgcccgcgcacgcgcactgggtgttggtcggagcgcttcttggccccgccaaacatcttgcttgggaagcgcaggccccagcctgtgttattgctgggcgatataaggatggacatgcttgctcaaaaagtgcggctcgataggacgcgcggcgagactatgcccagggccttgtaaacgtaggggcaggtgcggcgtctggcgtcagtaatggtcactcgctggactcctccgatgctgttgcgcagcggtagcgtcccgtgatctgtgagagcaggaacgttttcactgacggtggtgatggtgggggctggcgggcgcgccaaaatctggttctcgggaaagcgattgaacacgtgggtcagagaggtaaactggcggatgagttgggagtagacggcctggtcgttgtagaagctcttggagtgcacgggcaacagctcggcgcccaccaccggaaagttgctgatctggcgcgtggagcggaaggtcacggggtcttgcatcatgtctggcaacgaccagtagacctgctccgagccgcaggttacgtcaggagtgcaaagcagggtccatgagcggattccggtctgagggtcgccgtagttgtatgcaaggtaccagctgcggtactgggtgaaggtgctgtcattgcttattaggttgtaactgcgtttcttgctgtcctctgtcaggggtttgatcaccggtttcttctgaggcttctcgacctcgggttgcgcagcgggggcggcagcttcggccgctgcttcggcctcagcgcgcttctcctcagcccgtgtggcaaaggtgtcgccgcgaatggcatgatcgttcatgtcctccaccggctgcattgccgcggctgccgcgttggagttctcttccgcgccgctgccactgctgttgctgccgcctgcgccacccccgccctgttcggtgtcatctttcaagctcgcctggtaggcgtccacatccaacagtgcgggaatgttaccaccctccagatcatcgtaggtgatcctaaagccctcctggaagggttgccgcttgcggatgcccaacaagttgctcaggcggctgtgggtgaagtccaccccgcatcctggcagcaaaatgatgtctggatggaaggcttcgtttgtatataccccaggcatgacaagaccagtgacggggtcaaaccccagtctgaagttgcgggtgtcaaactttaccccgatgtcgctttccagaaccccgttctgtctgcccactttcaagtagtgctccacgatcgcgttgttcataaggtctatggtcatggtctcggagtagttgccctcgggcagcgtgaactccacccactcgtatttcagctccacctgattgtccttagtaggcaagcgcgacaccatcacccgcgccttaaacttattggtaaacatgaactcgttcacatttggcatgttggtatgcaggatggttttcaggtcgccgccccagtgcgaccggtcgtcaagattgatggtctgtgtgcttgcctcccccgggctgtagtcattgttttgaatgaccgtggtcagaaagttgctgtggtcgttctggtagttcagggatgccacatccgttgacttgttgtccaccaggtacacacgggtggtgtcgaataggggtgccaactcagagtaacggatgctgtttctccccccggtaggccgcaggtaccgcggaggcacaaacggcgggtccaggggagcatcgaagggagaacccagcgccgccgccactggcgccgcgctcaccacactctcgtaggagggaggaggaccttcctcatacatcgccgcgcgccgcatactaaggggaatacaagaaaaccaacgctcggtgccatggccttggtgagttttttattttgcatcatgcttttttttttttaaaacattctccccagcctggggcgaaggtgcgcaaacgggttgccactccctcccaaatccaggacgctgctgtcgtctgccgagtcatcgtcctcccacaccagaccccgctgacggtcgtgcctttgacgacgggtgggcgggcgcgggcctggcacgtccctgtgctcctgcgcgtacgtcttccatctactcatcttgtccactaggctctctatcccgttgttgggaaatgccggaggcaggtttttttcgcgctgcggctgcagcagcgagttgtttaggtactcctcctcgcccagcaggcgcgggcgggtggtgcgagtgctggtaagagaccctatcaagcttggaaatgggctactagcatctgaccgcggggccgcagcgcctagatcggacaagctgcttggcctgcggaagctttcctttcgcagcgccgcctctgcctgctcgcgctgttgcaactctagcagggtctgcggttgcggggaaaacacgctgtcgtctatgtcgtcccagaggaatccatcgttaccctcgggcacctcgaatcccccggtgtagaaaccagggggcggtagccagtgcgggttcaagatggcattggtgaaatactcggggttcacggcggccgcgcgatgcaagtagtccattaggcggttgataaacggccggtttgaggcatacatgcccggttccatgttgcgcgcggtcatgtccagcgccacgctgggcgttaccccgtcgcgcatcaggttaaggctcacgctctgctgcacgtagcgcaaaatgcgctcctcctcgctgtttaaactgtgcaacgaggggatcttctgccgccggttggtcagcaggtagtttagggttgcctccaggctgcccgtgtcctcctgccccagcgcgcggctgacacttgtaatctcctggaaagtatgctcgtccacatgcgcctgacctatggcctcgcggtacagtgtcagcaagtgacctaggtatgtgtcccgggacacgctgccactgtccgtgaagggcgctattagcagcagcaacaggcgcgagttgggcgtcagcaagctagacacggtcgcgcggtcgcctgtgggagcccgcaccccccacagcccctgcaagtttttgaaagcctggctcaggtttacggtctgcaggccttgtctactggtctggaaaaaatagtctggcccagactggtacacctcactttgcggtgtctcagtcaccattagccgcagtgcgctcacaaagttggtgtagtcctcctgtccccgcggcacgttggcgggctgtgtactcaggaaggcgtttagtgcaaccatggagcccaggttgccctgctgctgcgcgcgctcacgctgcgccacggcctcgcgcacatcccccaccagccggtccaggttggtctgcacgttgccgctgttgtaacgagccacgcgctgaagcagcgcgtcgtagaccaggccggcctcgtcgggccggatggccctgttttcggccagcgcgtttacgatcgccagcaccttctcgtgcgtggggtttgcgcgcgccgggaccaccgcttccagaattgcggagagccggttggcctgcggctgctgccggaacgcgtcaggattgcgcgcagtcagcgacatgatgcggtccatgacctggcgccagtcgtccgtggagttaaggccggacggctggctctgcagcgccgcccgcaccgccgggtccgttgcgtcttgcatcatctgatcagaaacatcaccgcttagtactcgccgtcctctggctcgtactcatcgtcctcgtcatattcctccacgccgccgacgttgccagcgcgcgcgggtgccaccgccagcccaggtccggccccagctgcctccagggcgcgtcggcttggggcccagcgcaggtcagcgcccgcgtcaaagtaggactcggcctctctatcgccgctgcccgtgccagccagggccctttgcaggctgtgcatcagctcgcggtcgctgagctcgcgccgccggctcacgctcacggccttgtggatgcgctcgttgcgataaacgcccaggtcgtcgctcaaggtaagcaccttcagcgccatgcgcatgtagaacccctcgatctttacctccttgtctatgggaacgtaaggggtatggtatatcttgcgggcgtaaaacttgcccaggctaagcatggaatagttgatggcggccaccttgtcagccaggctcaagctgcgctcctgcaccactatgctctgcaggatgtttatcaaatcgagcagccagcggccctcgggctctactatgtttagcagcgcatccctgaatgcctcgttgtccctgctgtgctgcactataaggaacagctgcgccatgagcggcttgctatttgggttttgctccagcgcgcttacaaagtcccacagatgcatcagtcctatagccacctcctcgcgcgccacaagcgtacgcacgtggttgttaaagcttttttgaaagttaatctcctggttcaccgtctgctcgtatgcggttaccaggtcggcggccgccacgtgtgcgcgcgcgggactaatcccggttcgcgcgtcgggctcaaagtcctcctcgcgcagcaaccgctcgcgattcaggccatgccgcagctcgcgccctgcgtggaactttcgatcccgcatctcctcgggctcctctccctcgcggtcgcgaaacaggttctgccgcggcacgtacgcctcacgcgtatcacgcttcagctgcacccttgggtgccgctcaggagagggcgctcctagccgcgccaggccctcgccctcctccaagtccaggtagtgccgggcccggcgccgcgggggttcgtaatcaccatctgctgccgcgtcaaccgcggatgtcgcccctcctgacgcggtaggaggaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggtccgagactcgaaccgggggtcccgcgactcaacccttggaaaataaccctccggctacagggagcgagccacttaatgctttcgctttccagcctaaccgcttacgctgcgcgcggccagtggccaaaaaagctagcgcagcagccgccgcgcctggaaggaagccaaaaggagcactcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacggtctagagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatatcatcgccttatgttggaagatctcgcccccggagccccggccaccctacgctggcccctctaccgccagccgccgccgcactttttggtgggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatggtgcttctgtcgaccatccgccgtctcaagaccgcctactttaattacatcatcagcagcacctccgccagaaacaaccccgaccgccgcccgctgccgcccgccacggtgctcagcctaccttgcgactgtgactggttagacgcctttctcgagaggttttccgatccggtcgatgcggactcgctcaggtccctcggcggcggagtacctacacaacaattgttgagatgcatcgttagcgccgtatccctgccgcatggcagccccccgccaacccataaccgggacatgacgggcggcgtcttccaactgcgcccccgcgagaacggccgcgccgtcaccgagaccatgcgccgtcgccgcggggagatgatcgagcgctttgtcgaccgcctcccggtgcgccgtcgtcgccgccgtgtcccccctcccccaccgccgccagaagaagaagaaggggaggcccttatggaagaggagattgaagaagaagaagaggcccctgtagcctttgagcgcgaggtgcgcgacactgtcgccgagctcatccgtcttctggaggaggagttaaccgtgtcggcgcgcaactcccagtttttcaacttcgccgtggacttctacgaggccatggagcgccttgaggccttgggggatatcaacgaatccacgttgcgacgctgggttatgtacttcttcgtggcagaacacaccgccaccaccctcaactacctctttcagcgcctgcgaaactacgccgtcttcgcccggcacgtggagctcaatctcgcgcaggtggtcatgcgcgcccgcgatgccgaagggggcgtggtctacagccgcgtctggaacgagggaggcctcaacgccttctcgcagctcatggcccgcatttccaacgacctcgccgccaccgtggagcgagccggacgcggagatctccaggaggaagagatcgagcagttcatggccgagatcgcctatcaagacaactcaggagacgtgcaggagattttgcgccaggccgccgtcaacgacaccgaaattgattctgtcgaactctctttcaggttcaagctcaccgggcccgtcgtcttcacgcagaggcgccagattcaggagatcaaccgccgcgtcgtcgcgttcgccagcaacctacgcgcgcagcaccagctcctgcccgcgcgcggcgccgacgtgcccctgccccctctcccggcgggtccggagccccccctacctccgggggctcgcccgcgtcaccgcttttagatgcatcatccaaggacacccccgcggcccaccgcccgccgcgcggtaccgtagtcgcgccgcggggatgcggcctcttgcaagccatcgacgccgccaccaaccagcccctggaaattaggtatcacctggatctagcccgcgccctgacccgtctatgcgaggtaaacctgcaggagctcccgcctgacctgacgccgcgggagctccagaccatggacagctcccatctgcgcgatgttgtcatcaagctccgaccgccgcgcgcggacatctggactttgggctcgcgcggcgtggtggtccgatccaccgtaactcccctcgagcagccagacggtcaaggacaagcagccgaagtagaagaccaccagccaaacccgccaggcgaggggctcaaattcccactctgcttccttgtgcgcggtcgtcaggtcaacctcgtgcaggatgtacagcccgtgcaccgctgccagtactgcgcacgtttttacaaaagccagcacgagtgttcggcccgtcgcagggacttctactttcaccacatcaatagccactcctccaattggtggcgggagatccagttcttcccgatcggctcgcatcctcgcaccgagcgtctctttgtcacctacgatgtagagacctatacttggatgggggcctttgggaagcagctcgtgcccttcatgctggtcatgaagttcggcggagatgagcctctagtgactgccgcgcgagacctagccgcgaaccttggatgggaccgctgggaacaagacccgcttaccttctactgcatcaccccagaaaaaatggccataggtcgccagtttaggacctttcgcgaccacctgcaaatgctaatggcccgtgacctgtggagctcattcgtcgcttccaaccctcatcttgcagactgggccctttcagagcacgggctcagctcccctgaagagctcacctacgaggaacttaaaaaattgccttccatcaagggcatcccgcgcttcttggaactttacattgtgggccacaacatcaacggctttgacgagatcgtgctcgccgcccaggtaattaacaaccgttccgaggtgccgggacccttccgcatcacacgcaactttatgcctcgcgcgggaaagatactcttcaacgatgtcaccttcgccctgccaaatccgcgttccaaaaagcgcacggactttttgctctgggagcagggcggatgcgacgacactgacttcaaataccagtacctcaaagtcatggtcagggacacctttgcgctcacccacacctcgctccggaaggccgcgcaggcatacgcgctacccgtagaaaagggatgctgcgcctaccaggccgtcaaccagttctacatgctaggctcttaccgttcggaggccgacgggtttccgatccaagagtactggaaagaccgcgaagagtttgtcctcaaccgcgagctgtggaaaaaaaagggacaggataagtatgacatcatcaaggaaaccctggactactgcgccctagacgtgcaggtcaccgccgagctggtcaacaagctgcgcgactcctacgcctccttcgtgcgtgacgcggtaggtctcacagacgccagcttcaacgtcttccagcgtccaaccatatcatccaactcacatgccatcttcaggcagatagtcttccgagcagagcagcccgcccgtagcaacctcggtcccgacctcctcgctccctcgcacgaactatacgattacgtgcgcgccagcatccgcggtggaagatgctaccctacatatcttggaatactcagagagcccctctacgtttacgacatttgcggcatgtacgcctccgcgctcacccaccccatgccatggggtcccccactcaacccatacgagcgcgcgcttgccgcccgcgcatggcagcaggcgctagacttgcaaggatgcaagatagactacttcgacgcgcgcctgctgcccggggtctttaccgtggacgcagaccccccggacgagacgcagctagaccccctaccgccattctgctcgcgcaagggcggccgcctctgctggaccaacgagcgcctacgcggagaggtagccaccagcgttgaccttgtcaccctgcacaaccgcggttggcgcgtgcacctggtgcccgacgagcgcaccaccgtctttcccgaatggcggtgcgttgcgcgcgaatacgtgcagctaaacatcgcggccaaggagcgcgccgatcgcgacaaaaaccaaaccctgcgctccatcgccaagttgctgtccaacgccctctacgggtcgtttgccaccaagcttgacaacaaaaagattgtcttttctgaccagatggatgcggccaccctcaaaggcatcaccgcgggccaggtgaatatcaaatcctcctcgtttttggaaactgacaatcttagcgcagaagtcatgcccgcttttcagagggagtactcaccccaacagctggccctcgcagacagcgatgcggaagagagtgaggacgaacgcgcccccacccccttttatagccccccttcaggaacacccggtcacgtggcctacacctacaaaccaatcaccttccttgatgccgaagagggcgacatgtgtcttcacaccctggagcgagtggaccccctagtggacaacgaccgctacccctcccacttagcctccttcgtgctggcctggacgcgagcctttgtctcagagtggtccgagtttctatacgaggaggaccgcggaacaccgctcgaggacaggcctctcaagtctgtatacggggacacggacagccttttcgtcaccgagcgtggacaccggctcatggaaaccagaggtaagaaacgcatcaaaaagcatgggggaaacctggtttttgaccccgaacggccagagctcacctggctcgtggaatgcgagaccgtctgcggggcctgcggcgcggatgcctactccccggaatcggtatttctcgcgcccaagctctacgccctcaaaagtctgcactgcccctcgtgcggcgcctcctccaagggcaagctgcgcgccaagggccacgccgcggaggggctggactatgacaccatggtcaaatgctacctggccgacgcgcagggcgaagaccggcagcgcttcagcaccagcaggaccagcctcaagcgcaccctggccagcgcgcagcccggagcgcaccccttcaccgtgacccagactacgctgacgaggaccctgcgcccgtggaaagacatgaccctggcccgtctggacgagcaccgactactgccgtacagcgaaagccgccccaacccgcgaaacgaggagatatgctggatcgagatgccgtagagcaggtgaccgagctgtgggaccgcctggaactgcttggtcaaacgctcaaaagcatgcctacggcggacggtctcaaaccgttgaaaaactttgcttccttgcaagaactgctatcgctgggcggcgagcgccttctggcggatttggtcagggaaaacatgcgagtcagggacatgcttaacgaagtggcccccctgctcagggatgacggcagctgcagctctcttaactaccagttgcagccggtaataggtgtgatttacgggcccaccggctgcggtaagtcgcagctgctcaggaacctgctttcttcccagctgatctcccctaccccggaaaccgttttcttcatcgccccgcaggtagacatgatccccccatctgaactcaaagcgtgggaaatgcaaatctgtgagggtaactacgcccctgggccggatggaaccattataccgcagtctggcaccctccgcccgcgctttgtaaaaatggcctatgacgatctcatcctggaacacaactatgacgttagtgatcccagaaatatcttcgcccaggccgccgcccgtgggcccattgccatcattatggacgaatgcatggaaaatcttggaggtcacaagggcgtctccaagttcttccacgcatttccttctaagctacatgacaaatttcccaagtgcaccggatacactgtgctggtggttctgcacaacatgaatccccggagggatatggctgggaacatagccaacctaaaaatacagtccaagatgcatctcatatccccacgtatgcacccatcccagcttaaccgctttgtaaacacttacaccaagggcctgcccctggcaatcagcttgctactgaaagacatttttaggcaccacgcccagcgctcctgctacgactggatcatctacaacaccaccccgcagcatgaagctctgcagtggtgctacctccaccccagagacgggcttatgcccatgtatctgaacatccagagtcacctttaccacgtcctggaaaaaatacacaggaccctcaacgaccgagaccgctggtcccgggcctaccgcgcgcgcaaaacccctaaataaagacagcaagacacttgcttgatccaaatccaaacagagtctggttttttatttatgttttaaaccgcattgggaggggaggaagccttcagggcagaaacctgctggcgcagatccaacagctgctgagaaacgacattaagttcccgggtcaaagaatccaattgtgccaaaagagccgtcaacttgtcatcgcgggcggatgaacgggaagctgcactgcttgcaagcgggctcaggaaagcaaagtcagtcacaatcccgcgggcggtggctgcagcggctgaagcggcggcggaggctgcagtctccaacggcgttccagacacggtctcgtaggtcaaggtagtagagtttgcgggcaggacggggcgaccatcaatgctggagcccatcacattctgacgcaccccggcccatgggggcatgcgcgttgtcaaatatgagctcacaatgcttccatcaaacgagttggtgctcatggcggcggcggctgctgcaaaacagatacaaaactacataagacccccaccttatatattctttcccacccttaagccacgcccacacatttcagtgatctgtcgaatcggatctgctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccgtttaaactcattactaaccggtacgcgtagaatcgagaccgaggagagggttagggataggcttaccttcgaaccgcgggccctctagatcaaccactttgtacaagaaagctgggtctaggatatctcagtgctagacagacgcggcaggtctgtattgttcagttaccacatcattatcaaaagtcgtggcagcagaagatttcttaccacacttactcattaaaggaataatgccaaagcatccacaacaacaaccacaacaaccagtcatgaaaaacaaccatccaagtattaagatgaagataatagtggcaaaagctatggctaacgccacatacgcaggcgccttaatataagttttgtacttccccagttcttgaaggtcgatcaggctctcgttcagattcttggccacctcgttcagccggtcgatctctttctggatgttcacgacgctggcattgattccgctgatgtcgcccaggtccacgtcggggcttgtgtggttcttaaagtacttatccagttcctctttgaagctgtccagctcgggctgcagagggtcgtacacggtattgttcacaatgccgatcacgacgtcgcagttgccagacacgaaggtgttgtcggtggtgatgatctggggctcgtagaagttccgctgggtcacgaaccaatgggtgccgttggacacgaacacgccttctctaggaaagtgggctttgccgtcgtggcagatggctggagcggtggtgaaattcttctcttgagcgggcacgtatgtcacgtgcagaaacaccacgccgtgaggggcagactgagggaagctcatcaggtggtagcccttgccgcaaaagtccactctcttgctctggcccagcacacactcagacatcttggtggcggccagattggcagaggctctaatctcggcggctctgatcagctgctgggtaacgtaggtctgcagggactgcagccttccggtgatcagtctgtcgatctgcacctcggcttccaccttgtccagtctgctcaggatatcgttcagcacagagctgatggcgccgaagttggaggacagctgcttgaccagggtgttcagtgcctgggcattctggttgaccacgtcctgcagctttcccagggcgcttgctgtgctgctcaggctgtcctggatcttgccgatggcgctgttgaactggttggcgatcagcttctggttctcgtacagcacattctgggtcactccgatgccgttgaaccggtaggccatctgcatagcaaaggggatctgcagagcggcgccagctccaaatgtccagccgcttgtgattgtgccggccagcagggcagatgtgtactgggcgatcatctcatcggtcagcagaggaggcagcactgtcagtccgttaaacttctgggcgcaaatcagatccctggcggcaatgtcgcccagacaatcgccatactgcttgatgaagccggcgtcggccagtgtcactttgttgaacagcaggtcctcgatgaagctccgcttgctgggcttgctaggatcgggcagaatctggctgaaattgaagccgccgaagtccttgataggaggggtcttgtagatctgcttcacttgggcgaacacctcttgggtgttcttgtcctgttccacggcgatccctgtcagggctctattcagctgggtgcagaagctgccgtactgcagcagcaggttggagcactcggtggaatcgccgcagatgtacatggtgcagtccacgctggtcttggtcatggacacaggcaggatctctgtggtcacgctgatggtgaagttggtggggatagcgatagagttgttggagtaggccacgctgttctcggcgcccagagacattgtgtaggcaatgatgctctggctggccacagatctggcccgtctggggctgtttgtctgtgtctggtagctggcacagatgccagcgccgatggggatgtcgcactcgtagctattgttcacgtgctcggctccgatcagacagccggctctggtctgaaacacattgctgccggtggagtacacccgccatgtaggtgtcagctgatcggcgtgaatggccacgggcacttcggtacagttcacgtcctggtacagcactgccacctgattgctggtgttggtgccaggggtgatcacagacactccgccgaagctgcaaggggtgatgtccaggatttccagtgtctggggatctctaacggcgtctgtggtatcggcgatatcccggccaaactgctggaatggcaggaacttcttgttgctctctgtcagcacgccggtgccggtcaggccgttgaagttgaagttcacgcatttgttcttcacgagattggtgcttttcttagggccgcacactgtggcaggggcatgcagcagttcgaagctcagcaccaccactctgtagggctgatagcccacgccatttgtgggctgaaagccgtaggactgcagtgggaagtagcagttgaagccttccacgccgttacaaggggtgctgccggcctgatagatctcggtggagatgtcccgctcgaagggcttcagattggacttccggaacagccggtacaggtaattgtagttgccgccgactttggagtccaggttgttgctgttccaggcaatcacacagccggtgaagtcgtcgggcagcttgtagttgtagtcggcgatcttgcctgtctgtccaggggcaatctgccgcacttcatctccccggatcacgaagctgtcggcgtacacgtttgtgaagcacaggtcgttcagcttggtaggggacacgccgtagcacttgaaggtgctgaagctggcggagttgtacagcacggagtagtcggccacgcaattgctgatccgcttccggttccaggcgtacacagaggcgaatctggtggcattgaacacctcgccgaaggggcacagattggtgatattggggaaccgcacgatggattcggtgggctgcacccggaagttgctggtctggtagatgcccttttccacggtgaaggacttcagggtgcactttgtctcgctcagaggatccagagcacaatccacggcgtcggtgatggtgccgttctcgttgtacttcagcaggaaggttctaggctgcaggtagcccacatagtaagcggcggcaccagctgtccatccgctgctgctatcgccaggtgtcaggtagcttctgtgcagggccagcagtgtctgaaaccgggtgatgttgatgccgatgggcagatccaccaggggttccagagcagagaagccctgaggcagatcccgcacgaggttgataggggtgtgcttgctgtagatcttgaagtagccgtcgatgttcttgaacacgaactcgcgcaggttcttgaagttgccctgcttgccttccaggtccatcaggaaaggctgggacacgtactcgaaggtgcagttgttggcgctgctgtacacccggaactcgctttccatccagctcttgttgttcttgtggtagtagacgcccaggaaggggtcgttgcagaactggaactcgcacactttgatgaccacgttggtggcgttgttcacgatcagcaggctctgggtcttgctgtccagtgtggtgccgaagatccagcctctgatgatgttggacttctcggtgctggcaaagtacaccccgtcgttgaagggcagcacggggttgtcgaatctcttggtgccattggtgccggacacgtggatggcgtggaaccaggtcacgttgctgaagaaaggcaggaacaggtcctgggtagagtgcagcacgctggatctgaacaccttgtcggggtagtacacgcctctggtaaagctgttggtgtaggctggaggcagctgtgttctggtggtcaggttcacacactggctggacaccagaggcagcagcaccagaaacacgaacatggtgggtacctcctttgaaagcctgcttttttgtacaaacttgttgatagcttaactagccagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcgggagatccgatgtcgctagcggccgctcgagaattctagaggtaccggatccaagcttcgactgactataataataaaacgccaactttgacccggaacgcggaaaacacctgagaaaaacacctgggcgagtctccacgtaaacggtcaaagtccccgcggccctagacaaatattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataaggtatattattgatgatg

SEQ ID NO: 40 - protein, 286 aa, Artificial Sequence
  Synthetic polypeptide
MSIQHFRVALIPFFAAFCLPVFAHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 41 - protein, 60 aa, Artificial Sequence
  Synthetic polypeptide
IEGRTQRRRHWRRAHHTLVGGRRTFLIHRRAPHTKGNTRKPTLGAMALVSFLFCIMLFFF

SEQ ID NO: 42 - protein, 74 aa, Artificial Sequence
  Synthetic polypeptide
YIKAPAYVALAIAFATIIFILILGWLFFMTGCCGCCCGCFGIIPLMSKCGKKSSAATTFDNDVVTEQYRPAASV

(19) United States
(12) Patent Application Publication
(10) Pub. No.: US 2024/0182528 A1
(43) Pub. Date: Jun. 6, 2024

(54) MODIFIED CHIMERIC CORONAVIRUS SPIKE PROTEIN FOR ENHANCEMENT OF VIRAL TITERS

(71) Applicant: iosBio Ltd, Haywards Heath (GB)

(72) Inventor: Jeffrey DREW, Haywards Heath (GB)

(21) Appl. No.: 18/552,338

(22) PCT Filed: Mar. 25, 2022
(86) PCT No.: PCT/GB2022/050759

§ 371 Date: Sep. 25, 2023

(30) Foreign Application Priority Data
Mar. 26, 2021 (GB) 2104320.3

(51) Int. Cl. C07K 14/005; A61K 39/00; A61K 39/215; A61P 31/14; C12N 15/86

(52) U.S. Cl. CPC C07K 14/005; A61K 39/215; A61P 31/14; C12N 15/86; A61K 2039/5256; A61K 2039/5258; C12N 2710/10334; C12N 2710/10343; C12N 2710/10352; C12N 2770/20022; C12N 2770/20034

(57) ABSTRACT

The disclosure relates to the production of viral particles, in particular adenoviral particles for use in vaccines. Modified chimeric coronavirus spike antigens are disclosed, together with polynucleotides encoding said antigens, viral particles and methods of production/formulation of the viral particles.

### FIELD OF THE INVENTION

The present invention relates to constructs and methods used to improve production of virus particles, especially virus particles expressing an antigen to be used in vaccines. The invention in particular relates to a modified antigen, where mutations have been made to the antigen in order to target the antigen to the plasma membrane and/or to reduce membrane fusion/syncytia formation.

For example, the invention relates to a modified form of the SARS-CoV-2 spike protein. The invention provides a polynucleotide encoding such a modified spike protein, together with virus particles comprising the polynucleotide and a pharmaceutical composition/vaccine comprising the viral particles. The invention also relates to a method of production of the viral particles and vaccine.

### BACKGROUND OF THE INVENTION

SARS-CoV-2 (also known as COVID-19 or 2019-nCoV) has been a significant global health problem since cases first emerged in late 2019. As of March 2021 there have been over 120,000,000 cases worldwide, and over 2,700,000 deaths. Vaccination against SARS-CoV-2 is seen as being critical in overcoming the pandemic.

A number of vaccines have been approved and others are in development, including viral vector vaccines, nucleic acid vaccines (mRNA and DNA), subunit vaccines, virus-like particle vaccines, inactivated vaccines and attenuated vaccines. Vaccines are typically utilising the SARS-CoV-2 spike protein as an antigen.

There remains a need to provide not only vaccines with enhanced immunogenicity, but also vaccines which can be produced more efficiently in order to meet worldwide demand. This is true not only for SARS-CoV-2, but also for vaccines targeting other viral infections.

### SUMMARY OF THE INVENTION

The invention provides a polynucleotide encoding an antigen from a virus, wherein the antigen has been modified (i) to target the antigen to the plasma membrane and/or (ii) to reduce membrane fusion and/or formation of syncytia.

The invention also provides a viral vector comprising a polynucleotide of the invention.

The invention further provides a polypeptide encoded by the polynucleotide of the invention and viral particles comprising the polynucleotide of the invention.

The invention also provides a vaccine comprising the viral particles.

Finally the invention provides a method of producing viral particles of the invention, said method comprising culturing host cells comprising a vector of the invention under conditions permitting production of the virus and harvesting the virus produced by the host cells

### BRIEF DESCRIPTION OF THE FIGURES

FIG. 1 shows an adenoviral vector of the invention encoding a modified SARS-CoV-2 spike protein of the invention.

FIG. 2 is a prism graph showing GEN1 and GEN3 yields (IFU/ml) in infected HEK-293 cells (each flask contained the same cell density and were infected with the same MOI, and were harvested at the same time). GEN1 and GEN3 titre was measured using AdenoX (Takara kit). Bars represent the mean titre from 3 AdenoX dilutions. N.B. AdenoX was performed in a 96-well plate format using a 10-fold dilution series.

FIG. 3 shows immunological responses (IgG) for GEN1 and GEN3 antigens.

### BRIEF DESCRIPTION OF THE SEQUENCE LISTING

SEQ ID NO: 1 shows the sequence of the SARS-CoV-2 spike protein from UniProt entry P0DTC2

SEQ ID NO: 2 shows the IBV canonical dilysine endoplasmic reticulum retrieval signal

SEQ ID NO: 3 shows the IBV cytoplasmic tail sequence

SEQ ID NO: 4 shows a coronavirus dibasic motif sequence

SEQ ID NO: 5 shows an IBV tail where the dilysine motif is replaced with alanines

SEQ ID NO: 6 shows an IBV tail where the dilysine motif is replaced with alanines and where the tyrosines in the internalisation motif are replaced with alanines

SEQ ID NO: 7 shows an exemplary cytoplasmic tail sequence

SEQ ID NO: 8 shows an exemplary cytoplasmic tail sequence

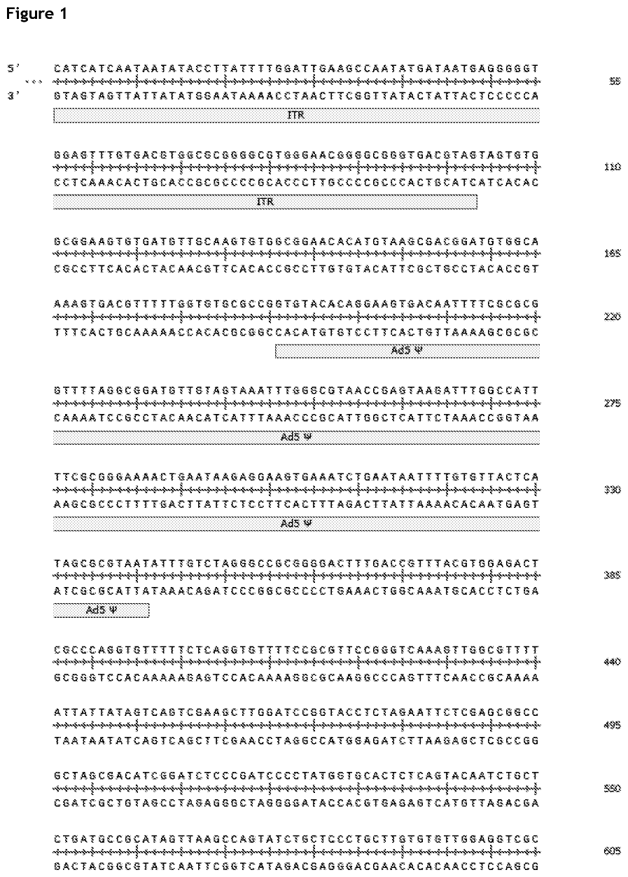

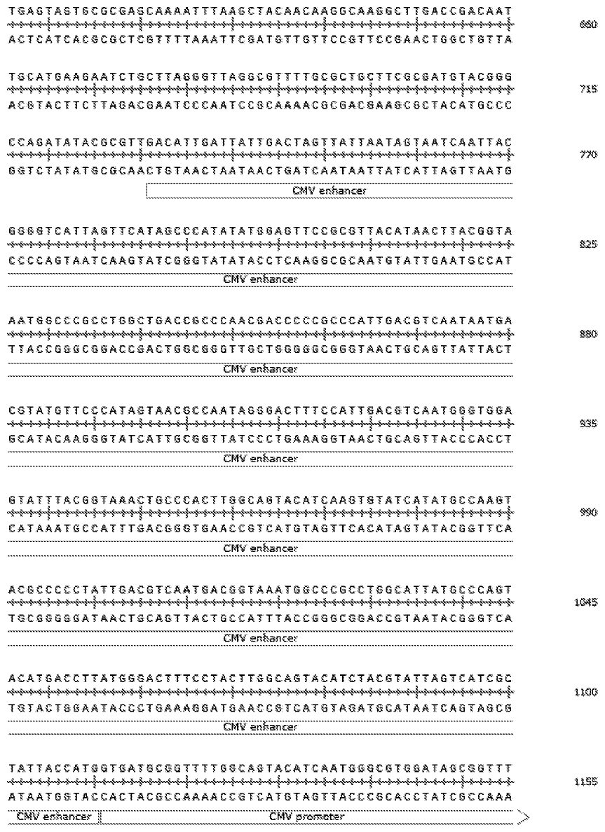

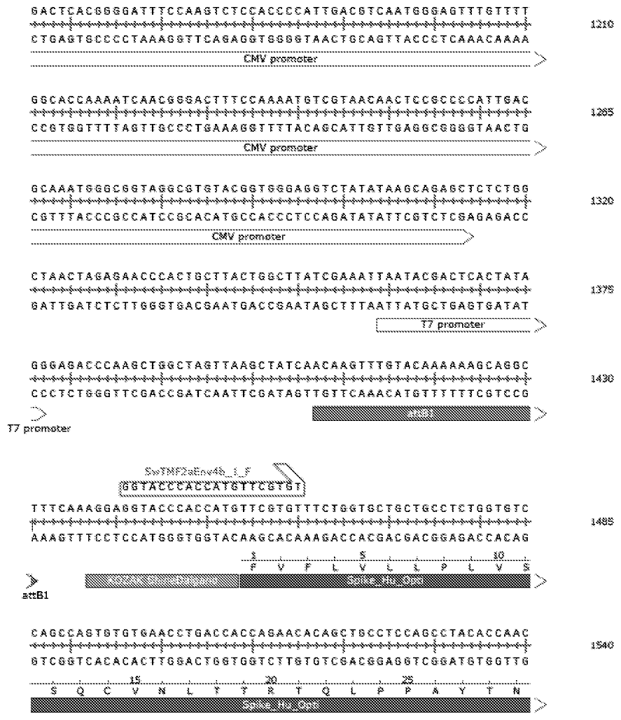

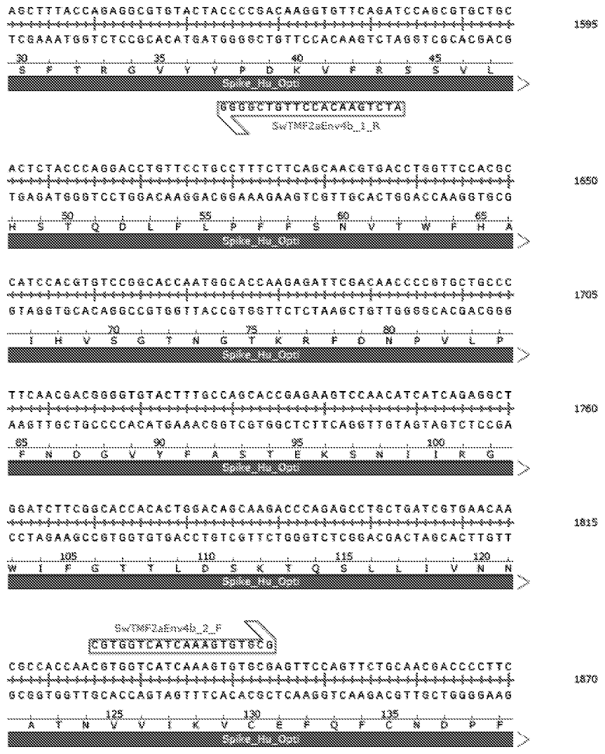

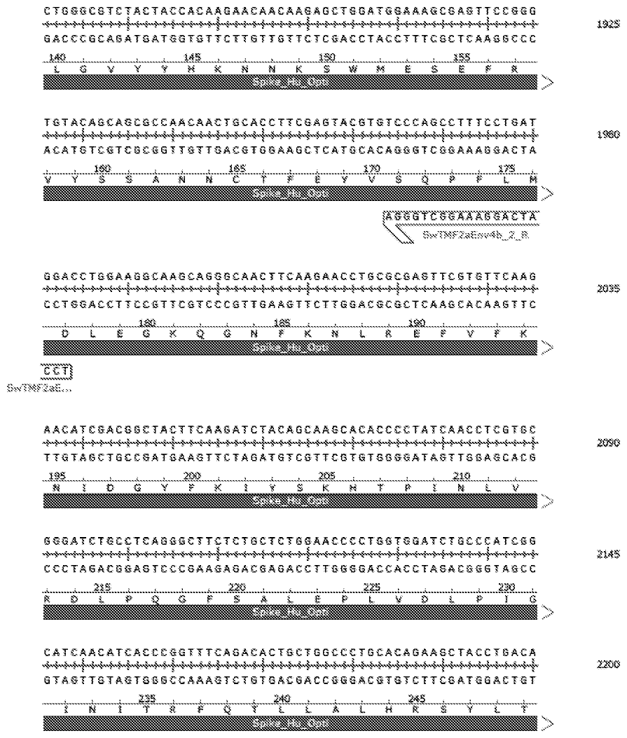

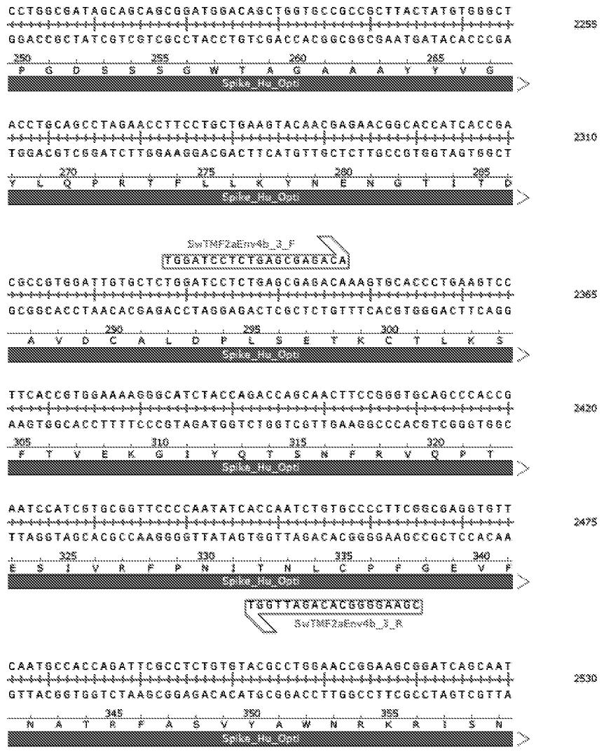

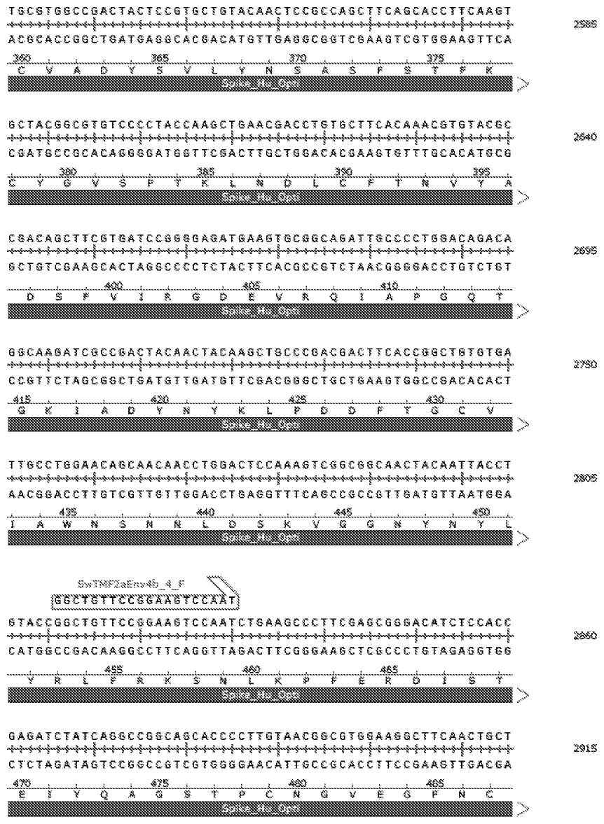

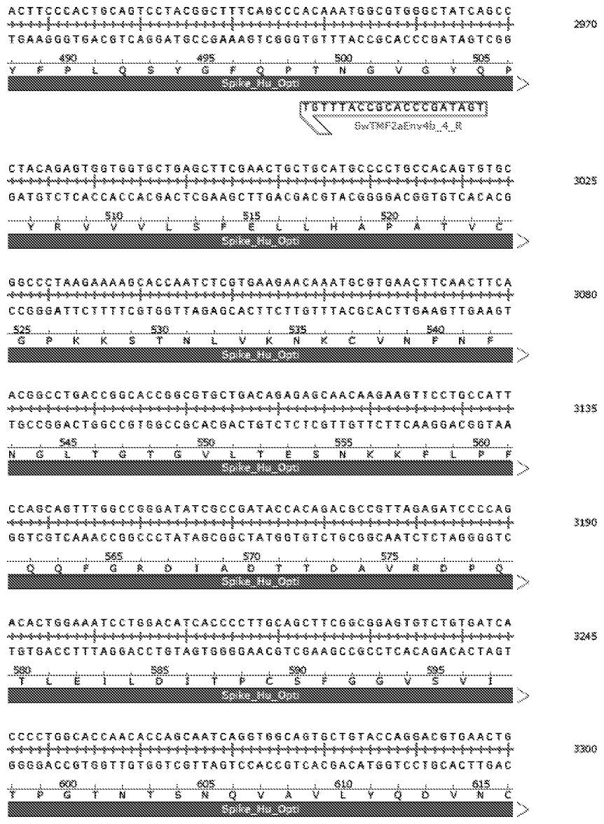

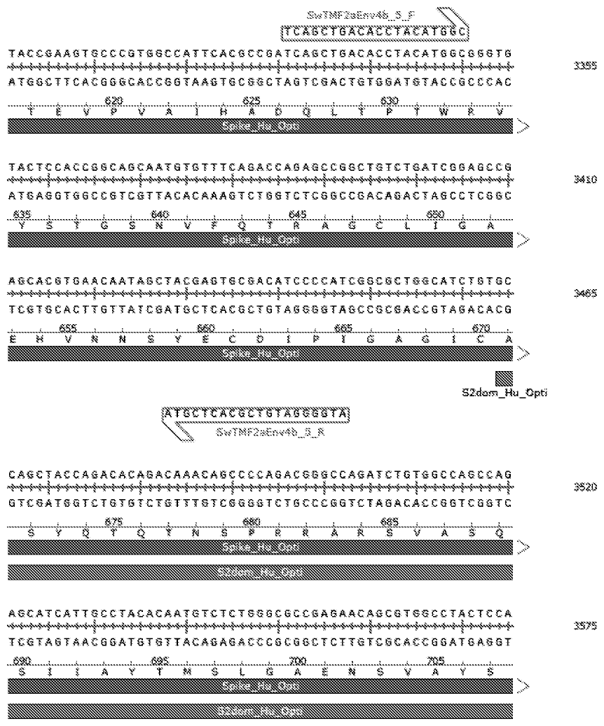

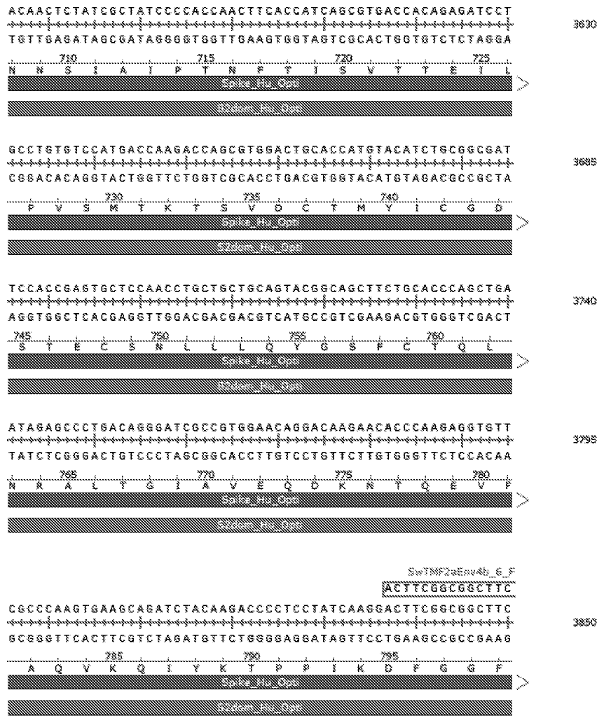

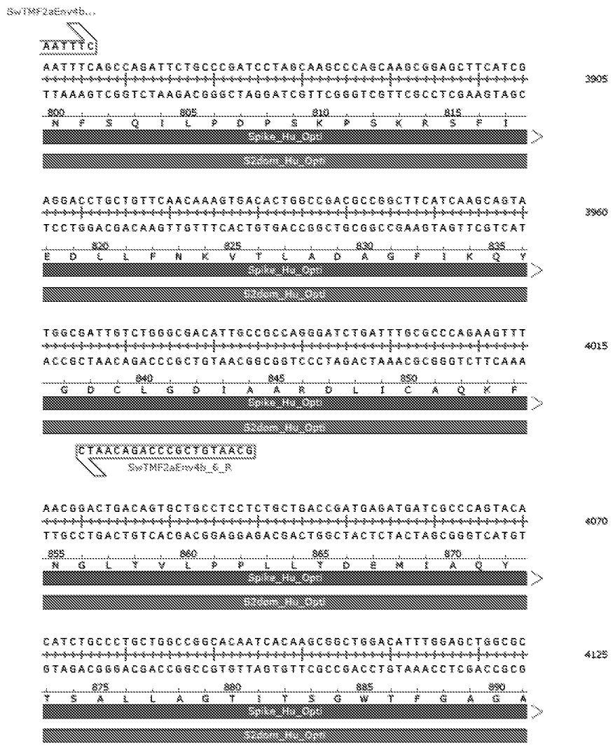

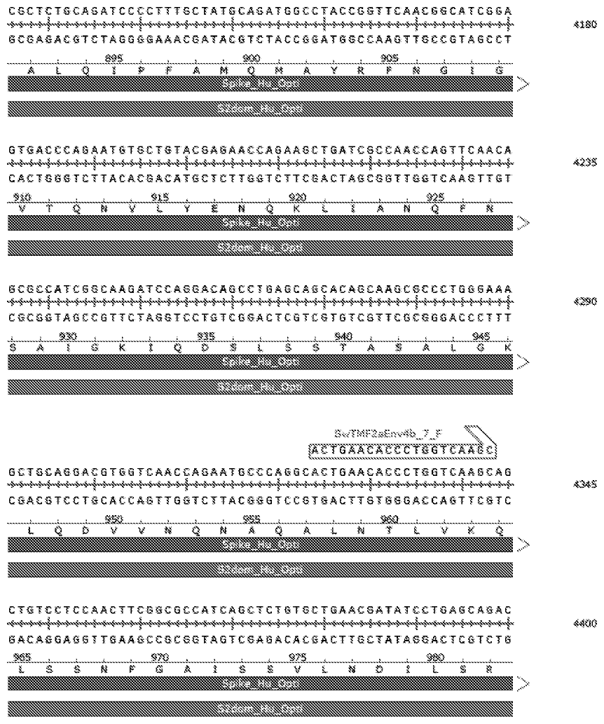

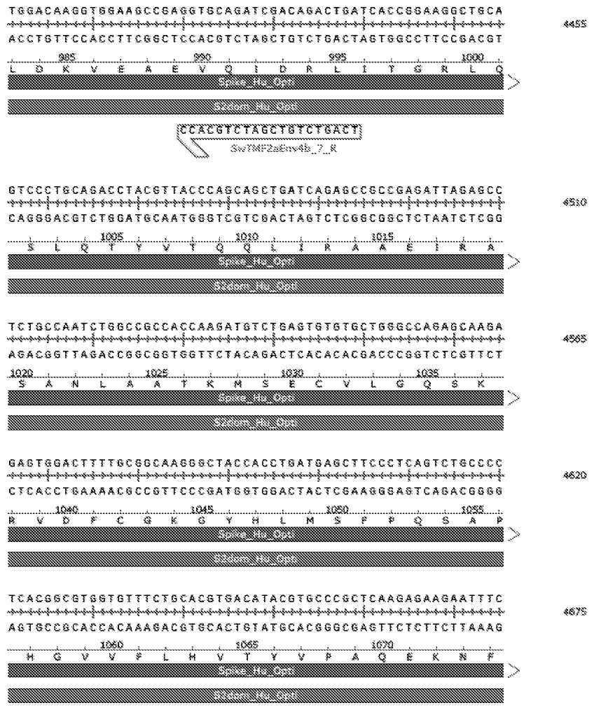

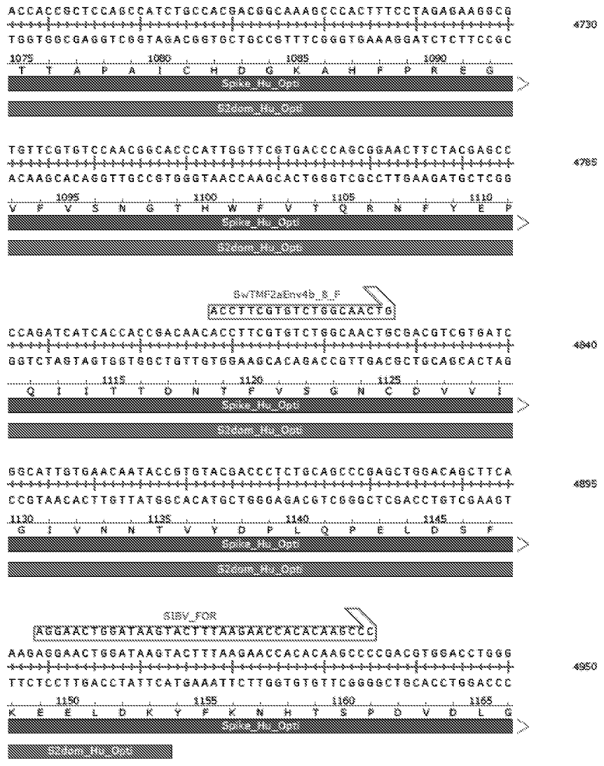

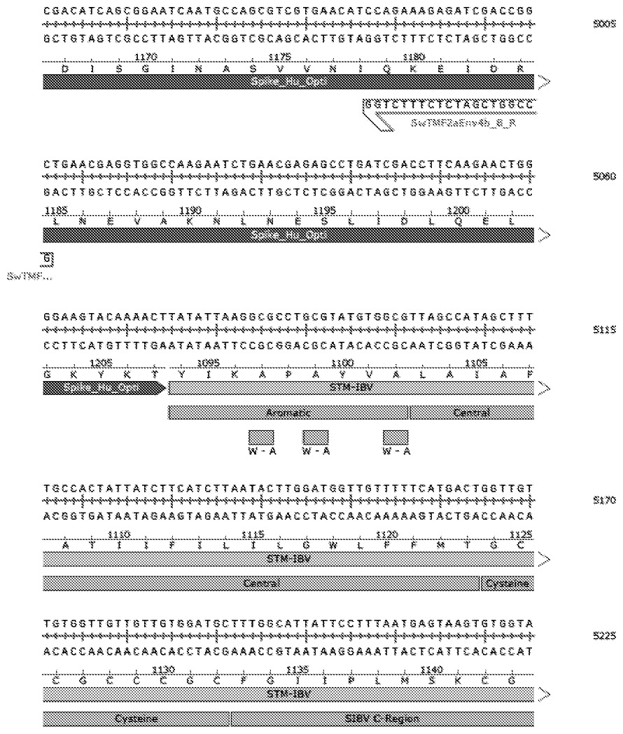

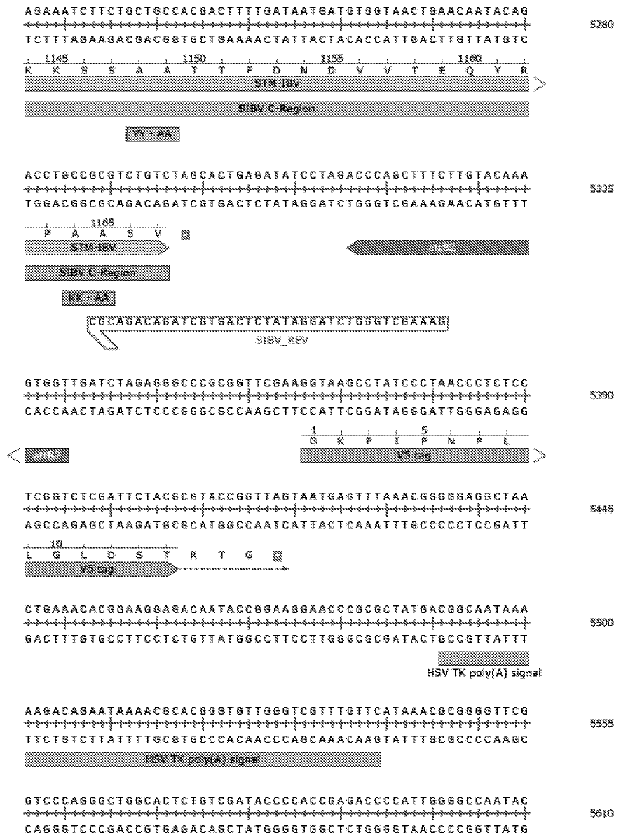

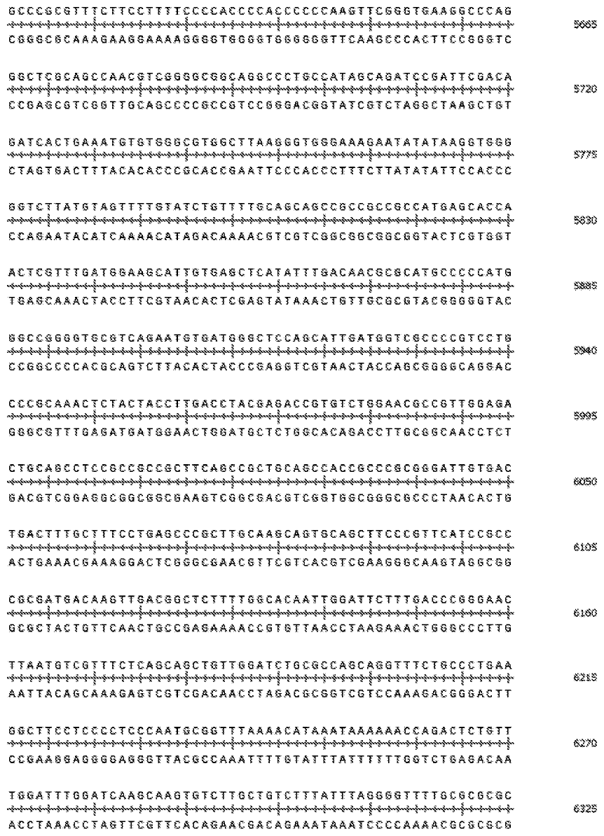

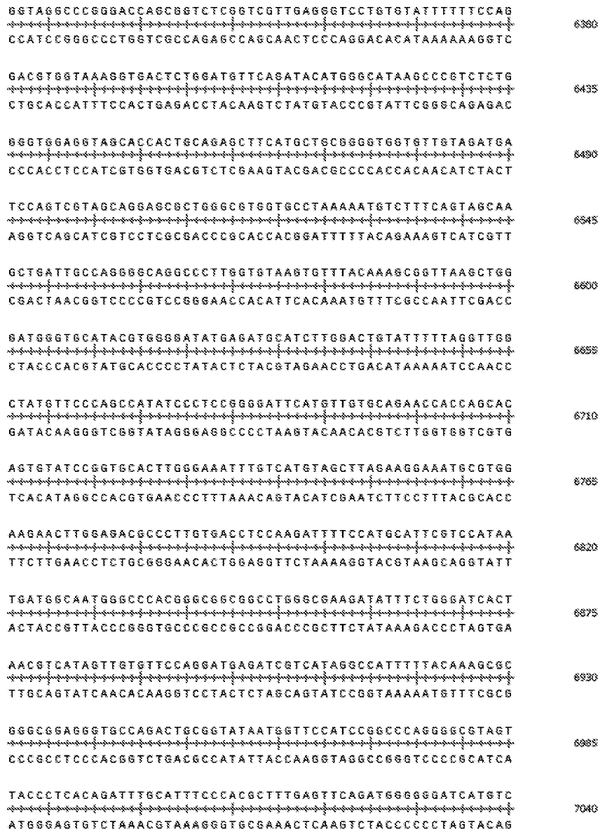

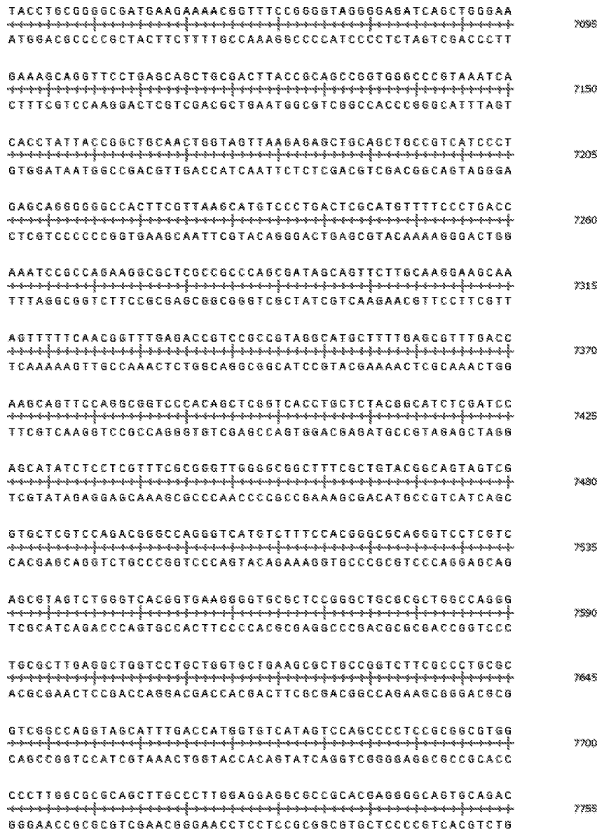

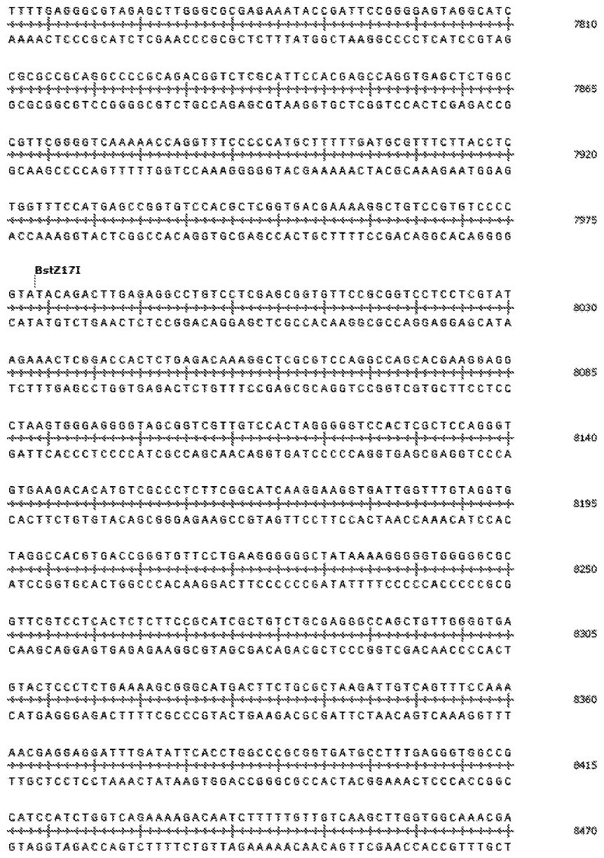

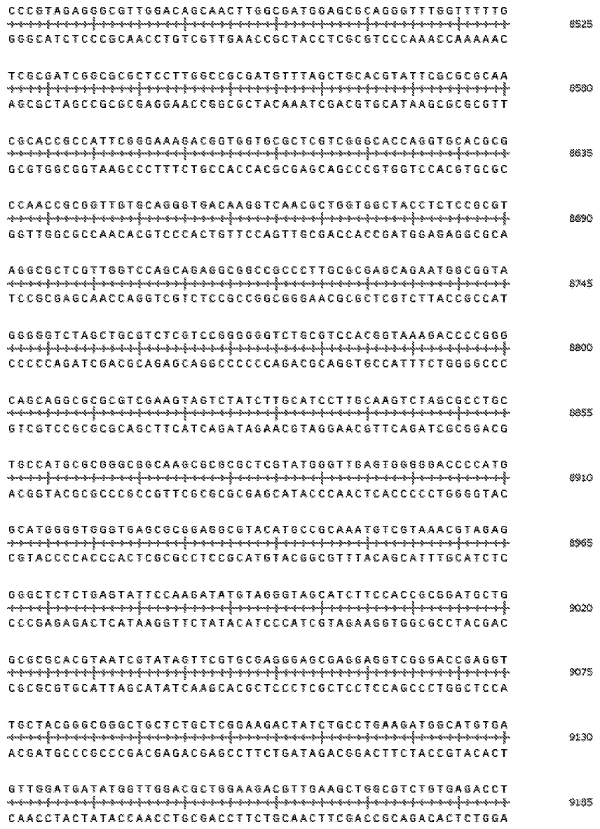

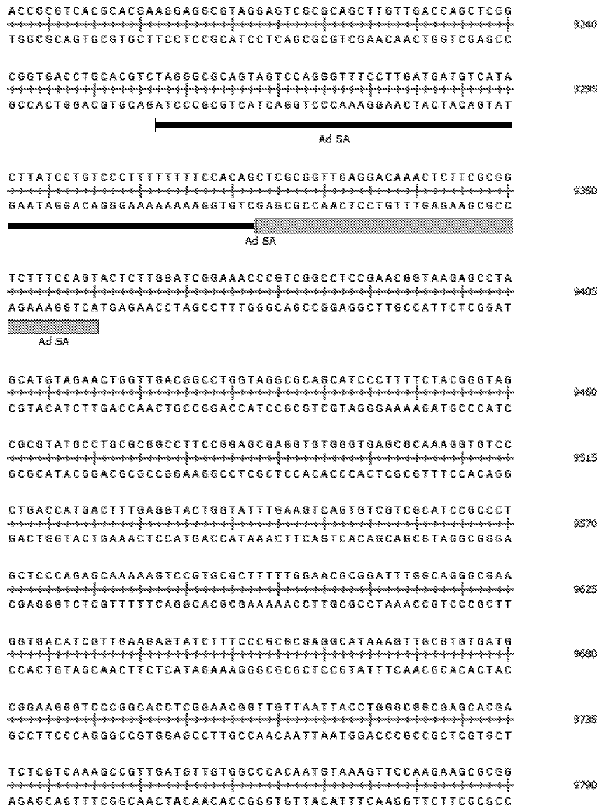

SEQ ID NO: 9 shows the complete vector sequence (nucleic acid) of FIG. 1

SEQ ID NO: 10 shows the aromatic stretch of SARS-CoV-2

SEQ ID NO: 11 shows an exemplary aromatic stretch

SEQ ID NO: 12 shows the aromatic stretch of SEQ ID NO: 11, where the tryptophans are replaced with alanine

SEQ ID NO: 13 shows an exemplary aromatic stretch

SEQ ID NO: 14 shows a modified version of SEQ ID NO: 13

SEQ ID NO: 15 shows a modified version of SEQ ID NO: 13

SEQ ID NO: 16 shows an exemplary sequence of the invention, with modifications to the cytoplasmic tail and transmembrane region

SEQ ID NO: 17 shows a SARS-CoV-2 spike protein sequence of the invention

SEQ ID NO: 18 shows the optimised SARS-CoV-2 spike protein sequence of FIG. 1 including the modified STM-IBV tail.

SEQ ID NO: 19 shows the SwTMF2aEnv4b_1_F sequence of FIG. 1

SEQ ID NO: 20 shows the SwTMF2aEnv4b_1_R sequence of FIG. 1

SEQ ID NO: 21 shows the SwTMF2aEnv4b_2_F sequence of FIG. 1

SEQ ID NO: 22 shows the SwTMF2aEnv4b_2_R sequence of FIG. 1

SEQ ID NO: 23 shows the SwTMF2aEnv4b_3_F sequence of FIG. 1

SEQ ID NO: 24 shows the SwTMF2aEnv4b_3_R sequence of FIG. 1

SEQ ID NO: 25 shows the SwTMF2aEnv4b 4_F sequence of FIG. 1

SEQ ID NO: 26 shows the SwTMF2aEnv4b_4_R sequence of FIG. 1

SEQ ID NO: 27 shows the SwTMF2aEnv4b_5_F sequence of FIG. 1

SEQ ID NO: 28 shows the SwTMF2aEnv4b_5_R sequence of FIG. 1

SEQ ID NO: 29 shows the SwTMF2aEnv4b_6_F sequence of FIG. 1

SEQ ID NO: 30 shows the SwTMF2aEnv4b_6_R sequence of FIG. 1

SEQ ID NO: 31 shows the SwTMF2aEnv4b_7_F sequence of FIG. 1

SEQ ID NO: 32 shows the SwTMF2aEnv4b_7_R sequence of FIG. 1

SEQ ID NO: 33 shows the SwTMF2aEnv4b_8_F sequence of FIG. 1

SEQ ID NO: 34 shows the SIBV_FOR sequence of FIG. 1

SEQ ID NO: 35 shows the SwTMF2aEnv4b_8_R sequence of FIG. 1

SEQ ID NO: 36 shows the SIBV_REV sequence of FIG. 1

SEQ ID NO: 37 shows the V5 tag of FIG. 1

SEQ ID NO: 38 shows the AmpR promoter and gene sequence of FIG. 1

SEQ ID NO: 39 shows the reverse strand of SEQ ID NO: 9

SEQ ID NO: 40 shows the polypeptide sequence of AmpR from FIG. 1

SEQ ID NO: 41 shows the Factor Xa site from FIG. 1

SEQ ID NO: 42 shows SEQ ID NO: 16, but without the initial KT residues

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise.

Thus, for example, reference to “an amino acid sequence” includes two or more such sequences, and the like.

All publications, patents and patent applications cited herein, whether supra or infra, are hereby incorporated by reference in their entirety.

### Polynucleotide and Polypeptide

The invention provides a polynucleotide encoding an antigen from a virus, wherein the antigen has been modified to target the antigen to the plasma membrane and/or to reduce membrane fusion/formation of syncytia. The virus may be any virus where infection proceeds through fusion of the viral membrane with the membrane of a target cell. The virus may be an enveloped virus. The virus may be a virus which forms syncytia.

In the invention the virus is typically a coronavirus (especially a SARS coronavirus, more particularly SARS-CoV-2). The virus could also be an orthomyxoviridae, a paramyxoviridae, a filoviridae, a retroviridae, a togaviridae, a rhabdoviridae, a arenaviridae, a flaviviridae, a bunyaviridae, a poxviridae, a hepadnaviridae or a herpesviridae. The virus could be, for example, influenza virus, respiratory syncytial virus (RSV), HIV, herpes simplex virus (HSV), Ebola virus, human T cell leukemia virus (HTLV), West Nile virus, Dengue fever virus or Measles morbillivirus (MeV). As discussed further below, the invention typically relates to SARS-CoV-2, although the modifications disclosed herein may be applied to other viruses.

Targeting of the antigen to the plasma membrane may be determined using techniques known in the art, for example fluorescent labelling of the antigen and visualisation. The presence of the antigen in the plasma membrane is increased when compared with the unmodified antigen. Likewise, membrane fusion or the formation of syncytia may be observed using standard techniques appropriate to the virus in question.

In the invention, as discussed below, targeting to the plasma membrane is typically increased by modifications in the cytoplasmic tail of the antigen and reductions in membrane fusion/the formation of syncytia are typically achieved by substituting tryptophan residues in the antigen. Such modifications are discussed below with respect to the SARS-CoV-2 spike protein. However, such modifications may equally be applied to antigens from other viruses.

The antigen is typically a viral membrane fusion protein. Three distinct classes of viral membrane fusion proteins have been identified based on structural criteria. The antigen could for example be a Class I, Class II or Class III fusion protein.

In some instances where the virus is an orthomyxoviridae the antigen could be the hemagglutinin (HA) antigen. For a retroviridae the antigen could be Env (a subunit thereof). For a paramyxoviridae the antigen could be F or HN. For coronoviridae the antigen could be the S protein. For filoviridae the antigen could be the GP protein. For arenaviridae the antigen could be GP or SSP. For togaviridae the antigen could be E1/E2. For Flaviviridae the antigen could be E or E1/E2. For bunyaviridae the antigen could be G_{N}/G_{C}. For rhabdoviridae the antigen could be G. For herpesviridae the antigen could be gB, gD or gH/L. For hepadnaviridae the antigen could be S or L.

The antigen may be RSF-V, influenza HA, West Nile Virus/Dengue fever E or HIV gp41.

In the invention, the antigen is typically a coronavirus spike antigen (S), preferably the SARS-CoV-2 spike antigen. Coronaviruses are enveloped viruses that assemble at intracellular membranes of the endoplasmic reticulum-Golgi intermediate compartment (ERGIC) in infected cells. Coronavirus are typically classified into groups 1, 2 or 3 by sequence homology.

Coronaviruses use the homotrimeric spike glycoprotein (S protein) on the envelope to bind to their cellular receptors. Such binding triggers a cascade of events that leads to the fusion between cell and viral membranes for cell entry. The SARS-CoV-2 spike protein is thought to bind to the ACE2 receptor on target cells.

Each monomer of the spike protein is thought to contain a subunit termed S1, which functions to bind the receptor on target cells. and a subunit termed S2, which functions to fuse the membranes of the virus and the host cell. The S1 region comprises an N-terminal domain (NTD) and a receptor binding domain (RBD). The S2 region then comprises a fusion peptide (FP), heptad repeats (HR1 and 2), a transmembrane domain (TM) and a cytoplasmic tail (CT). A protease cleavage site is present at the border between the S1 and S2 subunits.

The sequence of the SARS-CoV-2 spike protein from UniProt entry P0DTC2 is as follows:

(SEQ ID NO: 1)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

A number of variants of the spike protein have already been identified, including D614G (which may be associated with increased viral load). Other mutations include Q493N, Q493Y and N501T.

A summary of the main spike variants as of March 2022 is presented below:

| | | Country | Spike | | | |
|---|---|---|---|---|---|---|
| WHO | | first | mutations | Impact on | Impact on | Impact on |
| label | Lineage | detected | of interest | transmission | immunity | severity |
| Alpha | B.1.1.7 | UK | 69-70del, | Increased | Increased | Increased |
| | | | N501Y, | | | |
| | | | P681H | | | |
| Beta | B.1.351 | South | K417N, | Increased | Increased | Increased |
| | | Africa | E484K, | | | |
| | | | N501Y, | | | |
| | | | D614G, | | | |
| | | | A701V | | | |
| Gamma | P.1 | Brazil | K417T, | Increased | Increased | Increased |
| | | | E484K, | | | |
| | | | N501Y, | | | |
| | | | D614G, | | | |
| | | | H655Y | | | |
| Delta | B.1.617.2 | India | L452R, | Increased | Increased | Increased |
| | | | T478K, | | | |
| | | | D614G, | | | |
| | | | P681R | | | |
| Omicron | B.1.1.529 | South | (x) | Increased | Increased | Reduced |
| | | Africa and | | | | |
| | | Botswana | | | | |

x: A67V, Δ69-70, T95I, G142D, Δ143-145, N211I, Δ212, ins215EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F

Whilst the invention is in general described in relation to the above sequence, the modifications described below may equally be applied to other variants of the spike protein. Such variants may for example have 80, 90, 95, 96, 97, 98, or 99% amino acid sequence identity to the above sequence over the length of the sequence. Such variants may also have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acid substitutions, deletions or insertions relative to the above sequence (in addition to the changes described below).

Where residue numbering is provided for the above sequence, the skilled person would readily be able to identify corresponding residues of a variant sequence using standard techniques, such as use of sequence alignment algorithms.

In connection with amino acid sequences, “sequence identity” refers to sequences which have the stated value when assessed using ClustalW (Thompson et al., 1994, supra) with the following parameters:

Pairwise alignment parameters -Method: accurate, Matrix: PAM, Gap open penalty: 10.00, Gap extension penalty: 0.10;

Multiple alignment parameters -Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: G/P/S/N/D/Q/E/K/R. Sequence identity at a particular residue is intended to include identical residues which have simply been derivatized.

Previous studies have shown that coronavirus S proteins are concentrated in the Golgi region, although some S protein does appear to be transported to the cell surface.

The present inventors investigated modifications to the SARS-CoV-2 S protein in order to encourage cell membrane expression and retention (with the aim of increasing the immunogenicity). Mutations were initially made in the cytoplasmic tail.

Lontok et al (2004) Journal of Virology, 78, 5913-5922 investigates the intracellular targeting signals contributing to localisation of coronavirus spike proteins. In particular, Lontok et al demonstrated that the S protein from the group 3 coronavirus infectious bronchitis virus (IBV) contains a canonical dilysine endoplasmic reticulum retrieval signal (-KKXX-COOH; SEQ ID NO: 2) in its cytoplasmic tail. The IBV cytoplasmic tail has the sequence

GCCGCCCGCFGIPLMSKCGKKSSYYTFDNDVVIEQYRPKKSV (SEQ ID NO: 3). The dilysine motif is highlighted and a potential tyrosine internalisation signal is shown in italics. This dilysine motif causes retention of protein in the ERGIC, but when mutated allows transport of the S protein to the plasma membrane.

Such dibasic motifs have also been shown to exist in other types of coronavirus (e.g. KXHXX-COOH (SEQ ID NO: 4) in S proteins from group 1 coronaviruses).

As shown above the SARS-CoV-2 spike protein includes a KXHXX motif at the C terminus (positions 1269-1273).

The present invention provides a polynucleotide encoding a SARS-CoV-2 spike protein (or an antigen from an alternative virus), in which the cytoplasmic tail has been modified in order to direct the protein to the plasma membrane. For example, the polynucleotide may encode an antigen (SARS-CoV-2 spike protein) where a dibasic motif in the cytoplasmic tail is mutated to other amino acid residues. The dibasic motif is typically mutated to alanine residues, but could for example be a conservative substitution for alanine. For example, the dibasic motif could be mutated to valine, glycine, leucine or isoleucine (or any combination of A, V, G, L or I).

The dibasic motif can be the KXHXX motif of the above sequence, in which the K and H residues are mutated as described above, typically to alanine.

In some instances, the motif could also be e.g. a tribasic or polybasic motif with further basic amino acid residues. Basic amino acids are lysine, arginine and histidine.

For example, influenza hemagglutinin contains a tribasic motif. Such substitution could be to any amino acid residues which have the desired function. Such residues are typically mutated to alanine (or conservative alanine substitutions as described above).

In the invention, the polynucleotide preferably encodes an antigen (SARS-CoV-2 spike protein), where the cytoplasmic tail is replaced with the cytoplasmic tail from IBV and where the dilysine motif (at positions −3 and −4) is replaced with other amino acids. The amino acids are typically alanine residues, but could for example be conservative alanine substitutions as described above (in any combination). The sequence of such a cytoplasmic tail is shown below:

(SEQ ID NO: 5)
GCCGCCCGCFGIPLMSKCGKKSSYYTFDNDVVIEQYRPAASV

In some instances, the two tyrosine residues in the putative tyrosine internalisation sequence may also be mutated, in particular to alanine residues (or possibly any combination of V, G, L or I). A sequence with these modifications is shown below:

(SEQ ID NO: 6)
GCCGCCCGCFGIPLMSKCGKKSSAATFDNDVVIEQYRPAASV

In some instances, the cytoplasmic tail may comprise additional modifications relative to the sequences presented above (typically whilst retaining the alanine mutations described above). The modifications may be substitutions, deletions or insertions.

For example, the cytoplasmic tail may comprise up to 10 additional modifications or up to 5 additional modifications. In some instances, the cytoplasmic tail comprises 1, 2, 3, 4 or 5 modifications. In some instances, substitutions are conservative substitutions.

For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | | | |
|---|---|---|---|
| Ala | aliphatic, hydrophobic, | Met | hydrophobic, neutral |
| | neutral | | |
| Cys | polar, hydrophobic, neutral | Asn | polar, hydrophilic, neutral |
| Asp | polar, hydrophilic, charged | Pro | hydrophobic, neutral |
| | (−) | | |
| Glu | polar, hydrophilic, charged | Gln | polar, hydrophilic, neutral |
| | (−) | | |
| Phe | aromatic, hydrophobic, | Arg | polar, hydrophilic, charged |
| | neutral | | (+) |
| Gly | aliphatic, neutral | Ser | polar, hydrophilic, neutral |
| His | aromatic, polar, | Thr | polar, hydrophilic, neutral |
| | hydrophilic, charged (+) | | |
| Ile | aliphatic, hydrophobic, | Val | aliphatic, hydrophobic, |
| | neutral | | neutral |
| Lys | polar, hydrophilic, charged | Trp | aromatic, hydrophobic, |
| | (+) | | neutral |
| Leu | aliphatic, hydrophobic, | Tyr | aromatic, polar, hydrophobic |
| | neutral | | |

Amino acids used in the sequences of the invention may also be derivatized or modified, e.g. labelled.

In some instances, the cytoplasmic tail has at least 90% identity or at least 95% identity to the sequences shown above over the length of the sequence. Percentage identity is as described above. Once again, the alanine mutations described above are typically retained.

In some instances, the cytoplasmic tail comprises the following sequence:

(SEQ ID NO: 7)
GCCGCCCGCFGIIPLMSKCGKKSSAATTFDNDVVTEQYRPAASV
or
(SEQ ID NO: 8)
GCCGCCCGCFGIIPLMSKCGKKSSAATTFDNDVVTEQYRPAASVHHHHHH

The cytoplasmic tail may also be as shown in FIG. 1 (from position 1124-1167 in the STM-IBV).

In some instances, the cytoplasmic tail may comprise a variant of the above sequences (or the sequence as shown in FIG. 1). Such variants are as described above. Such variants typically retain the AA residues (or alternative substitutions) at positions −3 and −4 and the AA residues (or alternative substitutions) in the putative tyrosine internalisation sequence.

The polynucleotide of the invention may also encode additional mutations in the antigen, for example mutations designed to reduce/prevent membrane fusion and/or syncytial formation once the protein is present on the host cell surface and therefore enhancing antigen availability. Such mutations are described for SARS-CoV in Corver et al (2009), Virology Journal, 6.

As explained in Corver et al, the TM domain of the SARS-CoV spike protein consists of three domains: (1) a highly conserved N-terminal aromatic (tryptophan) rich stretch, (2) a hydrophobic core sequence and (3) a C-terminal cysteine rich domain. These domains are highly conserved in all coronaviruses. The aromatic stretch of the TM domain is in particular thought to be involved in entry of the virus into target cells and Corver et al investigated the effects of mutations in this region on membrane fusion and viral entry.

In the present invention, the polynucleotide may encode mutations in the antigen, especially in the TM domain of the antigen. These mutations may for example be in the aromatic stretch of the TM domain. The aromatic stretch of SARS-CoV-2 is present from residues 1209-1217 (YIKWPWYIW; SEQ ID NO: 10).

In some instances, the aromatic stretch is deleted. In other instances, the tryptophan residues in the aromatic stretch are replaced with other amino acid residues. This replacement amino acid may for example be phenylalanine. Preferably, the replacement amino acid is alanine (or a conservative substitution, such as V, G, L or I). All combinations are contemplated.

In some instances, a single tryptophan residue is mutated. Preferably at least two of the tryptophan residues are mutated. More preferably, all three of the tryptophan residues are mutated. As set out above, the replacement amino acid is preferably alanine but could be any appropriate amino acid (in some instances, V, G, L or I).

For example, as set out above, the aromatic stretch of SARS-CoV-2 is present from residues 1209-1217 of SEQ ID NO: 1 (YIKWPWYIW; SEQ ID NO: 10).

In some instances, a polynucleotide of the invention may encode a protein where residues 1209-1217 of the above sequence (SEQ ID NO: 1) are deleted.

In some instances, a polynucleotide of the invention encodes a W1212 mutation, typically a W1212A mutation (where the numbering is according to the SARS-CoV-2 sequence above). In some instances, a polynucleotide of the invention encodes a W1214 mutation, typically a W1214A mutation. In some instances, a polynucleotide of the invention encodes a W1217 mutation, typically a W1217A mutation. In some instances, a polynucleotide of the invention encodes mutations at two or the three positions (e.g. W1212A/W1214A, W1212A/W1217A or W1214A/W1217A). Typically, a polynucleotides encodes mutations at all three of these positions of the above sequence. (e.g. W1212A/W1214A/W1217A). As discussed above, the replacement amino acid is typically alanine but could be a conservative alanine substitution (all combinations are contemplated).

In the invention the aromatic stretch WXWXXW (SEQ ID NO: 11) may be replaced with AXAXXA (SEQ ID NO: 12) (or with conservative substitutions of A residues, in any combination). For example, the sequence WPWYIW (SEQ ID NO: 13) may be replaced with APAYIA (SEQ ID NO: 14) or in some instances APAYVA (SEQ ID NO: 15).

A polynucleotide of the invention may encode an antigen with the cytoplasmic tail sequences described above, and with the changes in the transmembrane domain described above. Furthermore, as also described above, other changes may be present.

In one instance, the antigen (especially a SARS-CoV-2 spike protein antigen) comprises the sequence

### KTYIKAPAYVALAIAFATIIFILILGWLFFMTGCCGCCCGCFGIIPLMSKCGKKSSAA TTFDNDVVTEQYRPAASV (SEQ ID NO: 16) or

YIKAPAYVALAIAFATIIFILILGWLFFMTGCCGCCCGCFGIIPLMSKCGKKSSAATT FDNDVVTEQYRPAASV (SEQ ID NO: 42). SEQ ID NO: 42 is the same as SEQ ID NO: 16, but without the initial KT residues. In some instances, the polynucleotide encodes the sequence from position 1094 in FIG. 1 (as labelled STM-IBV). In some instances, the antigen may also comprise a variant of these sequences. Such variants may for example have at least 90% identity; or be a variant with up to 10, in some instances up to 5, deletions, substitutions or additions (see above). The variant sequences typically retain the AXAXXA (SEQ ID NO: 12) motif (or alternative substitutions) described above and the AA residues (or alternative amino acids) at the −3 and −4 positions. The variant also typically retains the AA residues (or alternative amino acids) in the tyrosine internalisation sequence. Once again, the A residues could for example be A, V, G, L or I (in any combination).

In some instances, the polynucleotide of the present invention encodes a SARS-CoV-2 spike protein comprising the following sequence:

(SEQ ID NO: 17)
FVFLVLLPLVSSQCVNLTTRTQLPPAYTNSFTRGVYYPDKVFRSSVLHST
QDLFLPFFSNVTWFHAIHVSGTNGTKRFDNPVLPFNDGVYFASTEKSNII
RGWIFGTTLDSKTQSLLIVNNATNVVIKVCEFQFCNDPFLGVYYHKNNKS
WMESEFRVYSSANNCTFEYVSQPFLMDLEGKQGNFKNLREFVFKNIDGYF
KIYSKHTPINLVRDLPQGFSALEPLVDLPIGINITRFQTLLALHRSYLTP
GDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVY
AWNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYNY
LYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPG
TNTSNQVAVLYQDVNCTEVPVAIHADQLTPTWRVYSTGSNVFQTRAGCLI
GAEHVNNSYECDIPIGAGICASYQTQTNSPRRARSVASQSIIAYTMSLGA
ENSVAYSNNSIAIPTNFTISVTTEILPVSMTKTSVDCTMYICGDSTECSN
LLLQYGSFCTQLNRALTGIAVEQDKNTQEVFAQVKQIYKTPPIKDFGGFN
FSQILPDPSKPSKRSFIEDLLFNKVTLADAGFIKQYGDCLGDIAARDLIC
AQKFNGLTVLPPLLTDEMIAQYTSALLAGTITSGWTFGAGAALQIPFAMQ
MAYRFNGIGVTQNVLYENQKLIANQFNSAIGKIQDSLSSTASALGKLQDV
VNQNAQALNTLVKQLSSNFGAISSVLNDILSRLDKVEAEVQIDRLITGRL
QSLQTYVTQQLIRAAEIRASANLAATKMSECVLGQSKRVDFCGKGYHLMS
FPQSAPHGVVFLHVTYVPAQEKNFTTAPAICHDGKAHFPREGVFVSNGTH
WFVTQRNFYEPQIITTDNTFVSGNCDVVIGIVNNTVYDPLQPELDSFKEE
LDKYFKNHTSPDVDLGDISGINASVVNIQKEIDRLNEVAKNLNESLIDLQ
ELGKYKTYIKAPAYVALAIAFLTIIFILILGWVFFMTGCCGCCCGCFGII
PLMSKCSKKSSAATTFDNDVVTEQYRPAASV

In some instances, the polynucleotide encodes a spike protein as shown in FIG. 1 (SEQ ID NO: 18) (including the STM-IBV region).

In some instances, the polynucleotide encodes a spike protein with at least 90% identity to the sequence above, or the sequence in FIG. 1. Percentage identity is also described above. In other instances, the polynucleotide encodes a variant with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acid substitutions, deletions or insertions. In all cases, the variant sequences typically retain: the AXAXXA (SEQ ID NO: 12) motif, the

AA substitutions in the tyrosine internalisation sequence and the AA residues at the −3 and −4 positions of the sequence. Once again, substitutions other than A are also contemplated (see above).

Variant sequences of the invention preferably retain any advantageous properties of the parental sequence. For example, as discussed below, modified antigens of the invention preferably result in increased virus titres when compared with an unmodified sequence.

In some instances, a polynucleotide of the invention may be as shown in FIG. 1.

A polynucleotide of the invention may be RNA or DNA, but is typically DNA.

Polynucleotide sequences encoding an antigen of the invention can be determined by methods well known to those skilled in the art.

The invention also provides polypeptides encoded by polynucleotide sequences of the invention. For example, the invention provides a polypeptide of the spike protein sequence presented above, or the spike protein of FIG. 1. As described above, the polypeptide may also be a variant of these sequences.

### Vector and Virus Particles

The invention provides a viral vector comprising the polynucleotide of the invention, together with viral particles carrying the polynucleotide/vector.

The invention is typically performed using adenoviral particles/vectors. However, other suitable viruses are adeno-associated viruses, lentiviruses and oncolytic viruses such as HSV and Vaccinia viruses. The adenovirus is preferably a type 5 serotype, such as human serotype 5.

In a vector of the invention, a polynucleotide of the invention is inserted into a viral backbone vector (preferably an adenoviral vector). The adenoviral vector used in the invention may be either replication competent or replication competent. Such vectors are well known. For example, in a replication incompetent vector the E1 region may be deleted and replaced with an expression cassette with an exogenous promoter driving expression of the heterologous antigen. Usually, the E3 region is also deleted. Deletion of E3 allows for larger inserts into the E1 region. As discussed below, such vectors may be propagated in appropriate cell lines such as HEK 293 cells which retain and express the E1A and E1B proteins. Later generation vectors also lack the E4 region, and some vectors further lack the E2 region. E2 and E4 vectors must be grown on cell lines that complement the E1, E4 and E2 deletions.
Vectors may also be helper dependent vectors, which lack most or all of the adenoviral genes but retain cis-acting sequences such as the inverted terminal repeats as well as packaging sequences that are required for the genome to be packaged and replicated. These vectors are propagated in the presence of a helper adenovirus, which must be eliminated. Once again, such systems are well known in the art.
In the invention, the adenoviral vector is typically a replication incompetent vector (such as an E1/E3 deletion vector (ΔE1, ΔE3)), but the vector could also be helper-dependent or replication competent. Replication competent vectors are well known. The skilled person would readily be able to select an appropriate vector system.

Expression of the transgene can be driven via operable linkage to an appropriate promoter. Vectors used in the present invention may use other appropriate expression control elements, such as enhancers and regulators. Enhancers are broadly defined as a cis-acting agent, which when operably linked to a promoter/gene sequence, will increase transcription of that gene sequence. Enhancers can function from positions that are much further away from a sequence of interest than other expression control elements (e.g. promoters) and may operate when positioned in either orientation relative to the sequence of interest. Enhancers have been identified from a number of viral sources, including adenoviruses.

Adenoviral vectors may use high activity promoters such as the cytomegalovirus (CMV) promoter, SV40 large T antigen promoter, mouse mammary tumour virus LTR promoter, adenovirus major late promoter (MLP), the mouse mammary tumour virus LTR promoter or the SV40 early promoter.

Vectors of the invention may be produced using any appropriate method known in the art. For example a polynucleotide of the invention may be introduced into a vector using homologous recombination between the backbone vector and a second vector (e.g. a shuttle plasmid) comprising the polynucleotide. Such homologous recombination can be achieved using commercially available products, kits and protocols. In an alternative, a cassette carrying the polynucleotide of the invention can be directly inserted (cloned) into a vector without the need for homologous recombination. Again, such techniques are known in the art.

When the antigen is a SARS-CoV-2 spike protein, a vector of the invention may additionally encode other SARS-CoV-2 antigens, such as the SARS-CoV-2 nucleocapsid. A vector of the invention is shown in FIG. 1.

### Method of Production of Viral Particles

The invention also provides a method of preparing/producing the viral particles. The method comprises culturing host cells comprising a vector of the invention under conditions permitting production and replication of the virus and harvesting the virus produced by the host cells. The method may also comprise formulating the particles into a composition. The method also optionally comprises drying the composition, and packaging the composition into tablets of capsules for oral administration to a patient. These methods are described further below.

Vectors of the invention may be introduced into host cells using any appropriate technique. When the virus is an adenovirus, suitable cells include Human Embryonic Kidney (HEK) 293 cells, or HEK 293 derived cell clones, which are appropriate for the type of adenovirus being used. The cells may be cultured under any appropriate conditions that result in the production of virus particles. Culturing may involve an initial production phase, followed by an amplification phase.

Harvesting and purifying the virus may also be achieved by methods known in the art. For example, virus may be cultured in HEK 293 cells and purified by double caesium chloride density gradient centrifugation followed by column desalting (e.g. PD-10) into an excipient formulation. Excipient formulations are described below. The formulation may then be dried as described below.

The modified antigens of the invention preferably result in an increase in viral titre following culturing in the host cells compared with antigen which does not include the modifications of the invention. For example, the example of the present application shows that use of the modified SARS-CoV-2 spike protein of the invention resulted in a 40-fold increase in viral titre. Viral titres may be determined using methods known in the art.

In some instances, a modified antigen of the invention may result in a 10-, 20-, 30 or 40-fold increase in viral titre.

### Pharmaceutical Composition/Vaccine

The invention also provides a pharmaceutical composition comprising viral particles of the invention. The pharmaceutical composition is in particular a vaccine.

The pharmaceutical composition will normally be sterile and will typically include a pharmaceutically acceptable carriers and/or adjuvants. As used herein, “pharmaceutically acceptable carrier” includes any and all solvents, dispersion media, coatings, antibacterial and antifungal agents, isotonic and absorption delaying agents, and the like that are physiologically compatible. The pharmaceutical composition/vaccine may be suitable for parenteral, e.g. intravenous, intramuscular, intradermal, intraocular, intraperitoneal, subcutaneous, spinal or other parenteral routes of administration, for example by injection or infusion. Alternatively, the carrier may be suitable for non-parenteral administration, such as a topical, epidermal or mucosal route of administration. The carrier may in particular be suitable for oral administration.

Pharmaceutical compositions for the oral administration of viral particles are described in WO 2018/127702. As described in WO 2018/127702, the viral particles are formulated with excipients which provide thermal stability, including stabilising viral particles against damage caused by freezing, freeze drying and thawing.

In the present invention, the viral particles may be formulated in a composition which comprises a compound of formula (I) or a physiologically acceptable salt or ester thereof and/or a compound of formula (II) or a physiologically acceptable salt or ester thereof and optionally one, two or more sugars.

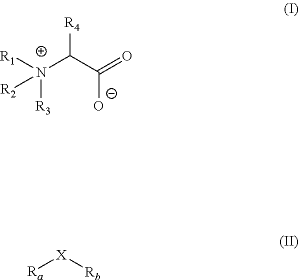

The viral particles are typically contacted with the compound of formula (I) or a physiologically acceptable salt or ester thereof and/or compound of formula (II) or a physiologically acceptable salt or ester thereof and optionally one or more sugars in an aqueous solution and the resulting solution in which the viral particles are present is then dried to form a composition incorporating the viral particles.

The viral particles may therefore be admixed with an aqueous solution of the compound of formula (I) or a physiologically acceptable salt or ester thereof and/or compound of formula (II) or a physiologically acceptable salt or ester thereof and optionally one or more sugars. The resulting solution is then dried to form a composition incorporating the viral particles. The dried composition may take the form of a cake or powder. The cake can be milled to a powder if required.

The viral particles are preserved in the aqueous solution prior to the drying step. This allows the aqueous solution to be stored after preparation, until such time as the drying step can be carried out, without undue loss of viral activity

The compounds of formula (I) and (II) may be present as a physiologically acceptable salt or ester thereof.

The salt is typically a salt with a physiologically acceptable acid and thus includes those formed with an inorganic acid such as hydrochloric or sulphuric acid or an organic acid such as citric, tartaric, malic, maleic, mandelic, fumaric or methanesulphonic acid. The hydrochloride salt is preferred.

The ester is typically a C₁₋₆ alkyl ester, preferably a C₁₋₄ alkyl ester. The ester may therefore be the methyl, ethyl, propyl, isopropyl, butyl, isobutyl or tert-butyl ester. The ethyl ester is preferred.

As used herein, a C₁₋₆ alkyl group is preferably a C₁₋₄ alkyl group. Preferred alkyl groups are selected from methyl, ethyl, propyl, isopropyl, butyl, isobutyl and tert-butyl. Methyl and ethyl are particularly preferred.

For the avoidance of doubt, the definitions of compounds of formula (I) and formula (II) also include compounds in which the carboxylate anion is protonated to give —COOH and the ammonium or sulfonium cation is associated with a pharmaceutically acceptable anion. Further, for the avoidance of doubt, the compounds defined above may be used in any tautomeric or enantiomeric form.

### Compounds of Formula (I)

Typically, R₁ represents hydrogen or C₁₋₆ alkyl and R₄ represents hydrogen. Typically, R₂ represents hydrogen or C₁₋₆ alkyl. Preferably, R₁ represents hydrogen or C₁₋₆ alkyl, R₄ represents hydrogen and R₂ represents hydrogen or C₁₋₆ alkyl. More preferably R₁ represents hydrogen or C₁₋₆ alkyl, R₄ represents hydrogen and R₂ represents C₁₋₆ alkyl.

Preferably, the compound of formula (I) is an N-C₁₋₆ alkyl-, N,N-di(C₁₋₆ alkyl)- or N,N,N-tri(C₁₋₆ alkyl)-glycine or physiologically acceptable salt or ester thereof, more preferably an N,N-di(C₁₋₆ alkyl)- or N,N,N-tri(C₁₋₆ alkyl)-glycine or physiologically acceptable salt or ester thereof. The alkyl group is typically a C₁₋₄ alkyl group. Preferred alkyl groups are selected from methyl, ethyl, propyl, isopropyl, butyl, isobutyl and tert-butyl. Methyl and ethyl are particularly preferred.

Preferred compounds of formula (I) are N-methylglycine, N,N-dimethylglycine or N,N,N-trimethylglycine or physiologically acceptable salts or esters thereof. N-Methyl-glycine is also called sarcosine. N,N-Dimethylglycine is also termed dimethylglycine (DMG) or 2-(dimethylamino)-acetic acid. N,N,N-trimethylglycine is termed trimethylglycine (TMG). The most preferred compound of formula (I) is DMG.

Alternatively, the compound of formula (I) is typically a glycine derivative of formula (IA) or a physiologically acceptable salt or ester thereof:

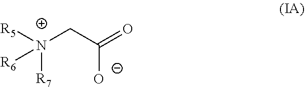

wherein R₅ and R₆ independently represent C₁₋₆ alkyl, for example C₁₋₄ alkyl such as methyl or ethyl; and Ry represents C₁₋₆ alkyl, for example C₁₋₄ alkyl such as methyl or ethyl, or —(CH₂)₂₋₅NHC(O)(CH₂)₅₋₁₅CH₃. Preferred compounds of formula (IA) are trimethylglycine (TMG) and cocamidopropyl betaine (CAPB) or physiologically acceptable salts or esters thereof. Trimethyglycine is preferred.

Alternatively, the compound of formula (I) is typically a proline derivative of formula (IB) or a physiologically acceptable salt or ester thereof:

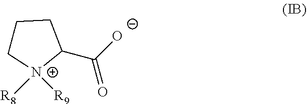

wherein R₈ and R₉ independently represent C₁₋₆ alkyl, for example C₁₋₄ alkyl such as methyl or ethyl. Preferably the compound of formula (IB) is an S-proline derivative. Preferably R₈ and R₉ both represent methyl; this compound is known as proline betaine. S-proline betaine or physiologically acceptable salt or ester thereof is particularly preferred:

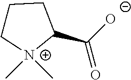

Compounds of formula (IA) or physiologically acceptable salts or esters thereof are preferred.

Preferably, the compound of formula (I) is N,N-dimethylglycine or N,N,N-trimethylglycine or physiologically acceptable salt or ester thereof. Most preferably, the compound of formula (I) is N,N-dimethylglycine or physiologically acceptable salt or ester thereof.

### Compounds of Formula (II)

Typically, the carboxylate and amine substituents of R_{c} are attached to the same carbon atom of the R_{c} alkyl moiety. Typically R_{c} is a C₂₋₄ or C₂₋₃ alkyl moiety.
The compound of formula (II) is typically a sulfone compound of formula (IIA) or a physiologically acceptable salt or ester thereof:

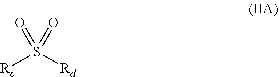

wherein R_{c} and R_{d} independently represent C₁₋₆ alkyl, for example C₁₋₄ alkyl. Preferred alkyl groups are selected from methyl, ethyl, propyl, isopropyl, butyl, isobutyl and tert-butyl. Methyl and ethyl are particularly preferred. The most preferred sulfone compound is methylsulfonylmethane (MSM), which is also known as dimethylsulfone (DMSO₂).

The compound of formula (II) may be a compound of formula (IIB) or a physiologically acceptable salt or ester thereof:

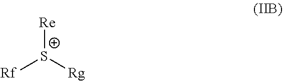

wherein Rₑ and R_{f} independently represent C₁₋₆ alkyl, for example C₁₋₄ alkyl such as methyl or ethyl, and R_{g} represents C₁₋₆ alkyl, for example C₁₋₄ alkyl such as methyl or ethyl, substituted with a carboxylate anion and with an amine (—NH₂) moiety. Preferably the carboxylate and amine substituents are attached to the same carbon atom. A preferred compound of formula (IIB) is S-methyl-L-methionine (SMM) or a physiologically acceptable salt or ester thereof.

In the present invention, it is most preferred that the compound of formula (I) is DMG or a physiologically acceptable salt or ester thereof and the compound of formula (II) is MSM or a physiologically acceptable salt or ester thereof.

### Sugars

Sugars suitable for use in the present invention include reducing sugars such as glucose, fructose, glyceraldehydes, lactose, arabinose and maltose; and preferably non-reducing sugars such as sucrose and raffinose, more preferably sucrose. The sugar may be a monosaccharide, disaccharide, trisaccharide, or other oligosaccharides. The term “sugar” includes sugar alcohols. In one embodiment, therefore, use of a non-reducing sugar or a sugar alcohol is preferred.
Monosaccharides such as galactose and mannose; dissaccharides such as sucrose, lactose and maltose; trisaccharides such as raffinose; and tetrasaccharides such as stachyose are envisaged. Trehalose, umbelliferose, verbascose, isomaltose, cellobiose, maltulose, turanose, melezitose and melibiose are also suitable for use in the present invention. A suitable sugar alcohol is mannitol. When mannitol is used, cakes of improved appearance can be obtained on freeze-drying.

The presence of sugar may act to improve stability. The addition of sugar may also provide other benefits such as an altered lyophilisation cake and improved solubility for faster reconstitution. Generally one or more sugars is present when freeze-drying is used. When one sugar is used, the sugar is preferably sucrose or mannitol.

Preservation of viral activity is particularly effective when two or more sugars are used in the preservation mixture. Two, three or four sugars may be used. Preferably, the aqueous solution is a solution of sucrose and raffinose. Sucrose is a disaccharide of glucose and fructose. Raffinose is a trisaccharide composed of galactose, fructose and glucose.

In the present invention, the compound of formula (I) is preferably DMG or a physiologically acceptable salt or ester thereof and the compound of formula (II) is preferably MSM or a physiologically acceptable salt or ester thereof. The composition preferably also comprises sucrose.

### Other Components of the Aqueous Composition

In the present invention, an aqueous solution comprising the viral particles, optionally one or more sugars and a compound of formula (I) or a physiologically acceptable salt or ester thereof and/or a compound of formula (II) or a physiologically acceptable salt or ester thereof is typically dried. Any suitable aqueous solution may be used. The solution may be buffered. The solution may be a HEPES, phosphate-buffered, Tris-buffered or pure water solution.

The solution may have a pH of from 2 to about 12 and may be buffered. The solution may be buffered with HEPES buffer, phosphate-buffer, Tris-buffer, sodium citrate buffer, bicine buffer (i.e. N,N-bis(2-hydroxyethyl) glycine buffer) or MOPS buffer (i.e. 3-(N-morpholino) propanesulfonic acid buffer). The solution may or may not contain NaCl. The solution may thus be a saline sodium citrate (SSC) buffered solution.

Generally a preparation of the viral particles is admixed with the preservation mixture, i.e. with an aqueous solution of a compound of formula (I) or a physiologically acceptable salt or ester thereof and/or a compound of formula (II) or a physiologically acceptable salt or ester thereof and optionally one, two or more sugars. The preservation mixture may itself be buffered. It may be a HEPES, phosphate-buffered, Tris-buffered or pure water solution.

Alternatively, the aqueous solution may typically consist, or consist essentially, of viral particles, a compound of formula (I) or a physiologically acceptable salt or ester thereof and/or a compound of formula (II) or a physiologically acceptable salt or ester thereof, and optionally one or more sugars.

The concentrations of the compound of formula (I) or a physiologically acceptable salt or ester thereof and/or a compound of formula (II) or a physiologically acceptable salt or ester thereof and of each optional sugar can be determined by routine experimentation. Optimised concentrations which result in the best stability can thus be selected. The compound of formula (I) or a physiologically acceptable salt or ester thereof and/or a compound of formula (II) or a physiologically acceptable salt or ester thereof compound may act synergistically to improve stability.

The concentration of sugar when present in the aqueous solution for drying is at least 0.01M, typically up to saturation. Generally the sugar concentration when present is at least 0.1M, at least 0.2M or at least 0.5M up to saturation e.g. saturation at room temperature or up to 3M, 2.5M or 2M. The sugar concentration may therefore range from, for example, 0.1M to 3M or 0.2M to 2M. Preferably a sugar is present. Alternatively, the sugar concentration or the total sugar concentration if more than one sugar is present may therefore range from 0.08M to 3M, from 0.15M to 2M or from 0.2M to 1M. A suitable range is from 0.05 to 1M.

When more than one sugar is present, preferably one of those sugars is sucrose. The sucrose may be present at a concentration of from 0.05M, 0.1M, 0.25M or 0.5M up to saturation e.g. saturation at room temperature or up to 3M, 2.5M or 2M.

The ratio of the molar concentration of sucrose relative to the molar concentration of the other sugar(s) is typically from 1:1 to 20:1 such as from 5:1 to 15:1. In the case when two sugars are present and in particular when sucrose and raffinose are present, therefore, the ratio of molar concentrations of sucrose is typically from 1:1 to 20:1 such as from 5:1 to 15:1 and preferably about 10:1.

The concentration of each compound of formula (I) or physiologically acceptable salt or ester thereof or compound of formula (II) or physiologically acceptable salt or ester thereof in the aqueous solution for drying is generally in the range of from 0.001M to 2.5M and more especially from 0.01M to 2.5M. For example, the concentration range may be from 0.1M to 2.5M.

Alternatively, for example when the compound of formula (I) is DMG or a salt or ester, the concentration of each compound of formula (I) or physiologically acceptable salt or ester thereof or compound of formula (II) or physiologically acceptable salt or ester thereof in the aqueous solution for drying is generally in the range of 0.1 mM to 3M or from 1mM to 2M. The concentration may be from 1 mM to 1.5M or from 5 mM to 1M or from 0.07M to 0.7M. Preferred concentrations are from 7 mM to 1.5M or from 0.07M to 1.2M. Another further preferred range is 0.5 to 1.5M, particularly when the compound of formula (I) is an N-alkylated glycine derivative such as DMG.

The particular concentration of compound of formula (I) or physiologically acceptable salt or ester thereof or compound of formula (II) or physiologically acceptable salt or ester thereof that is employed will depend on several factors including the type of viral particle to be preserved; the particular compound being used; whether one, two more sugars are present and the identity of the sugar(s); and the drying procedure and conditions. Thus:
The concentration of a compound of formula (II) in which X represents —S(O)₂— or a compound of formula (IIA), such as MSM, or a physiologically acceptable salt or ester thereof is preferably from 0.2 mM to IM such as from 0.35 mM to 1M, from 3.5mM to 0.5M, from 0.035M to 0.5M or from 0.035M to 0.25M.
The concentration of a compound of formula (I) or a compound of formula (IA) or formula (IB), such as TMG, or a physiologically acceptable salt or ester thereof is preferably used at a concentration from 0.01M to 2M such as from 0.07M to 2M, from 0.2M to 1.5M, from 0.23M to 1.5M or from 0.07M to 0.7M.

The concentration of a compound of formula (II) in which X represents —S⁺(R_{c})— or a compound of formula (IIB), such as S-methyl-L-methionine, or a physiologically acceptable salt or ester thereof is preferably from 0.005M to 2M such as from 0.007M to 2M, from 0.02M to 2M, from 0.023M to 1.5M or from 0.07M to 1M.
The concentration of a compound of formula (I), such as N,N-dimethylglycine (DMG) or a physiologically acceptable salt or ester thereof, when no sugar is present are from 5 mM to 1.5M or from 70 mM to 1.5M or to 1.2M or from 7 mM to 1M. More preferred concentrations are from 0.023M to 0.7M or 1M, or from 0.07M to 0.7M or 1M, such as about 0.7M
The concentration of a compound of formula (I), such as N,N-dimethylglycine (DMG) or a physiologically acceptable salt or ester thereof, when one or more sugars are present are generally lower and in the range of from 1 mM to 1M or 1.5M or from 5 mM to 1M. More preferred concentrations are from 0.007M to 0.7M or 1M such as about 0.007M. A particularly preferred range is 0.5 to 1.5M.

When a compound of formula (I) or physiologically acceptable salt or ester thereof and a compound of formula (II) or physiologically acceptable salt or ester thereof are present, and preferably when an N-alkylated glycine derivative or salt or ester thereof and a sulfone compound of formula (IIA) or (IIC) are present, the compounds can be present in amounts that result in synergy. For example:
The concentration of the N-alkylated glycine derivative or salt or ester thereof in the aqueous solution for drying is generally in the range of 0.1 mM to 3M or from 1 mM to 2M. The concentration may be from 1 mM to 1.5M or from 5 mM to 1M. Preferred concentrations are from 0.1M to 1.5M or from 0.5M to 1.25M.
The concentration of the sulfone compound of formula (IIA) or (IIC) in the aqueous solution for drying is generally in the range of 0.1 mM to 3M, from 1 mM to 2M or from 0.2 mM to 1M. The concentration may be from 0.1M to 1.5M or from 0.5M to 1.25M.

The composition may also comprise other preservatives such as antioxidants, lubricants and binders well known in the art.

The composition may be dried as described below.

### Drying

Typically, drying is achieved by freeze drying, vacuum drying, fluid bed drying or spray-drying. Freeze-drying is preferred. By reducing the water in the material and sealing the material in a vial, the material can be easily stored, shipped and later reconstituted to its original form. The drying conditions can be suitably optimized via routine experimentation.

On drying, a composition is formed which incorporates the viral particles. A matrix incorporating the viral particles is produced. The composition is typically an amorphous solid. A solid matrix, generally an amorphous solid matrix, is thus generally formed. By “amorphous” is meant non-structured and having no observable regular or repeated organization of molecules (i.e. non-crystalline).

The sugar or sugars when present provide the amorphous matrix in the dried composition. The compound of formula (I) or a physiologically acceptable salt or ester thereof and/or a compound of formula (II) or physiologically acceptable salt or ester thereof is dispersed in the sugar matrix. The compound of formula (I) or a physiologically acceptable salt or ester thereof and/or compound of formula (II) or physiologically acceptable salt or ester thereof is thus incorporated within the sugar matrix. The viral particles are incorporated within the sugar matrix too. The drying procedure can thus be effected e.g. by freeze-drying to form an amorphous cake within which the viral particles are incorporated.

The drying step is generally performed as soon as the aqueous solution has been prepared or shortly afterwards. Alternatively, the aqueous solution is typically stored prior to the drying step. The viral particle in the aqueous solution is preserved by the compound of formula (I) or a physiologically acceptable salt or ester thereof and/or a compound of formula (II) or physiologically acceptable salt or ester thereof and, optionally, one or more sugars during storage.

The aqueous solution, or bulk intermediate solution, is generally stored for up to 5 years, for example up to 4 years, 3 years, 2 years or 1 year. Preferably the solution is stored for up to 6 months, more preferably up to 3 months or up to 2 months, for example 1 day to 1 month or 1 day to 1 week. Prior to drying, the solution is typically stored in a refrigerator or in a freezer. The temperature of a refrigerator is typically 2 to 8° C., preferably 4 to 6° C., or for example about 4° C. The temperature of a freezer is typically −10 to −80° C., preferably −10 to −30° C., for example about −20° C.

The solution is typically stored in a sealed container, preferably a sealed inert plastic container, such as a bag or a bottle. The container is typically sterile. The volume of the bulk intermediate solution is typically 0.1 to 100 litres, preferably 0.5 to 100 litres, for example 0.5 to 50 litres, 1 to 20 litres or 5 to 10 litres. The container typically has a volume of 0.1 to 100 litres, preferably 0.5 to 100 litres, for example 0.5 to 50 litres, 1 to 20 litres or 5 to 10 litres.

If the stored bulk intermediate solution is to be freeze-dried, it is typically poured into a freeze-drying tray prior to the drying step.

There are three main stages to freeze-drying namely freezing, primary drying and secondary drying. Freezing is typically performed using a freeze-drying machine. In this step, it is important to cool the biological material below its eutectic point, (Teu) in the case of simple crystalline products or glass transition temperature (Tg′) in the case of amorphous products, i.e. below the lowest temperature at which the solid and liquid phase of the material can coexist. This ensures that sublimation rather than melting will occur in the following primary drying stage.

During primary drying the pressure is controlled by the application of appropriate levels of vacuum whilst enough heat is supplied to enable the water to sublimate. At least 50%, typically 60 to 70%, of the water in the material is sublimated at this stage. Primary drying may be slow as too much heat could degrade or alter the structure of the biological material. A cold condenser chamber and/or condenser plates provide surfaces on which the water vapour is trapped by resolidification.

In the secondary drying process, water of hydration is removed by the further application of heat. Typically, the pressure is also lowered to encourage further drying. After completion of the freeze-drying process, the vacuum can either be broken with an inert gas such as nitrogen prior to sealing or the material can be sealed under vacuum.

In certain embodiments, vacuum drying is carried out using vacuum desiccation at around 1300 Pa. However vacuum desiccation is not essential to the invention and in other embodiments, the preservation mixture contacted with the viral particle is spun (i.e. rotary desiccation) or freeze-dried. Advantageously, the method of the invention further comprises subjecting the preservation mixture containing the viral particle to a vacuum. Conveniently, the vacuum is applied at a pressure of 20,000 Pa or less, preferably 10,000 Pa or less. Advantageously, the vacuum is applied for a period of at least 10 hours, preferably 16 hours or more. As known to those skilled in the art, the period of vacuum application will depend on the size of the sample, the machinery used and other parameters.

In another embodiment, drying is achieved by spray-drying or spray freeze-drying the viral particles admixed with the preservation mixture of the invention. These techniques are well known to those skilled in the art and involve a method of drying a liquid feed through a gas e.g. air, oxygen-free gas or nitrogen or, in the case of spray freeze-drying, liquid nitrogen. The liquid feed is atomized into a spray of droplets. The droplets are then dried by contact with the gas in a drying chamber or with the liquid nitrogen.

In a further embodiment, drying is achieved by fluid bed drying the viral particles admixed with the preservation mixture. This technique is well known to those skilled in the art and typically involves passing a gas (e.g. air) through a product layer under controlled velocity conditions to create a fluidized state. The technique can involve the stages of drying, cooling, agglomeration, granulation and coating of particulate product materials. Heat may be supplied by the fluidization gas and/or by other heating surfaces (e.g. panels or tubes) immersed in the fluidized layer. Cooling can be achieved using a cold gas and/or cooling surfaces immersed in the fluidized layer. The steps of agglomeration and granulation are well known to those skilled in the art and can be performed in various ways depending on the product properties to be achieved. Coating of particulate products such as powders, granules or tablets can be achieved by spraying a liquid on the fluidized particles under controlled conditions.

A composition having a low residual moisture content can therefore be obtained. A level of residual moisture content is achieved which offers long term preservation at greater than refrigeration temperatures e.g. within the range from 40° C. to 56° C. or more, or lower than refrigeration temperatures e.g. within the range from 0 to −70° C. or below. The dried composition may thus have residual moisture content of 10% or less, 5% or less, 2% or less or 1% or less by weight. Preferably the residual moisture content is 0.5% or more 1% or more. Typically a dried composition has residual moisture content of from 0.5 to 10% by weight and preferably from 1 to 5% by weight.

The composition can be obtained in a dry powder form. A cake resulting from e.g. freeze-drying can be milled into powder form. A solid composition according to the invention thus may take the form of free-flowing particles.

In the invention, a powder may be compressed into tablet form. Tablets are described below. The powder may also be filled into capsules. Once again, capsules are described below.

### Tablets and Capsules

The pharmaceutical compositions described herein may be administered in the form of an aqueous suspension or solution or troche, but are typically administered as a tablet or capsule. The compositions may also be administered as gelatin wafers. Tablets may be coated or un-coated. Preferably, the composition is incorporated into a capsule, such as a gelatine capsule.

Pharmaceutically compatible binding agents, and/or adjuvant materials can be included in oral formulations. The tablets, capsules, troches and the like can contain any of the following ingredients, or compounds of a similar nature: a binder such as microcrystalline cellulose, gum tragacanth or gelatine; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, or flavouring.

Capsules and tablets are typically enterically coated. The skilled person would readily be able to select and apply an appropriate enteric coating depending on the transgene to be delivered using methods known in the art. For example, the enteric coating may target delivery to the duodenum. An example of such a coating is poly(methacyclic acid-co-thyl acrylate) 1:1 copolymer. The enteric coating may have a threshold of pH 5.8-6.8

### Method of Therapy

The invention also comprises methods of therapy comprising administering the viral particles, or a pharmaceutical composition comprising the viral particles, to a patient in need thereof. The patient is typically a human, but could also be an animal such as a domestic, companion (such as a dog or cat) or livestock animal (sheep, pigs, cows).

Administration of the viral particles may be for either prophylactic or therapeutic purposes. The viral particles of the invention are, however, typically used in a vaccine which is intended to prevent a disease occurring, or reduce the severity of such a disease. In the case of a viral infection, prevention of disease typically means that the infection is prevented from occurring in a patient. A reduction in severity of disease could mean that the patient is asymptomatic for the disease. or a reduction in the severity of symptoms is observed.

Doses and frequency of dosing will depend on the disease and the desired outcome and can be readily ascertained by the skilled person.

The invention in particular provides methods for preventing SARS-CoV-2 infection, or reducing the severity of SARS-CoV-2 infection, said method comprising administering viral particles or a composition/vaccine of the invention to a patient in need thereof.

The invention also provides viral particles/vaccines of the invention for use in the manufacture of a medicament for preventing SARS-CoV-2 infection, or reducing the severity of SARS-CoV-2 infection.

Furthermore, the invention provides viral particles or a vaccine of the invention for use in a method for preventing SARS-CoV-2 infection, or reducing the severity of SARS-CoV-2 infection.

In all of these cases, the viral particles will express a SARS-CoV-2 antigen, in particular a S protein as described above.

The following examples are presented below so as to provide those of ordinary skill in the art with a complete disclosure and description of how to make and use the methods and compositions of the invention. The examples are not intended to limit the scope of what the inventors regard as their invention.

### EXAMPLES

Experiments were performed using SARS-CoV-2 unmodified spike protein (“GEN1”) and with a modified spike protein with the sequence as shown in FIG. 1 (“GEN3”). Adenoviral vectors (serotype 5, ΔE1, ΔE3) were prepared and introduced into HEK293 cells.

### Example 1—Viral Yields

Plated HEK-293 cells were infected with adenoviral vectors encoding GEN1 and GEN3 spike protein (0.1 ppc-2 T175 flasks per virus) for bulking up (and for comparison of virus yields):

The following was observed:
GEN3=>0.1 ppc×10×10⁶ cells=10×10⁵ particles/5.4×10⁷ pfu/ml=18.5 μl
GEN1=>0.1 ppc×10×10⁶ cells=10×10⁵ particles/1.0×10⁸ pfu/ml=10 μl
GEN3 and GEN1 infected T175 flasks of HEK-293 cells were checked (0.1 ppc 96 h). For both GEN3 and GEN1 more areas of CPE were observed. Each flask was topped up with another 10 ml DMEM.

Two days later, the GEN3/GEN1 infected HEK-293 cells were checked again. More plaques/CPE had formed and more cells had detached from flask. GEN3 and GEN1 infected HEK-293 cells were then harvested by centrifugation at 4100rpm for 1 hour at 4° C., re-suspension of cell pellets in 4 ml supernatant. The remaining supernatant was kept and stored at −80° C.

Titres of harvested GEN1 and GEN3 viruses were then tested using AdenoX. Initially, a serial dilution of samples was produced by adding to 20 μl stock to DMEM (10⁻¹ dilution to 10⁻¹² dilution). 100 μl of each dilution was added to the HEK-293 cells and incubated at 37° C. for 48 h.

Results were as follows:
GEN3 (HEK-293):
2 (10⁻⁷ dilution)=>2 particles in 100 μl (100 μl of 10⁻⁷ dilution was added to 100 μl of HEK-293 cells for AdenoX)=>2×10⁷ particles in 100 μl of original sample=>2×108 pfu/ml
4 (10⁻⁶ dilution)=>4 particles in 100 μl (100 μl of 10⁻⁶ dilution was added to 100 μl of HEK-293 cells for AdenoX)=>4×10⁶ particles in 100 μl of original sample=>4×10⁷ pfu/ml
20 (10⁻⁵ dilution)=>20 particles in 100 μl (100 μl of 10⁻⁵ dilution was added to 100 μl of HEK-293 cells for AdenoX)=>20×10⁵ particles in 100 μl of original sample=>20×10⁶ pfu/ml
Mean GEN3 titre=>8.67×10⁷ pfu/ml
GEN1 (HEK-293):
5 (10⁻⁵ dilution)=>5 particles in 100 μl (100 μl of 10⁻⁵ dilution was added to 100 μl HEK-293 cells for AdenoX)=>5×10⁵ particles in 100 μl of original sample=>5 10⁶ pfu/ml
11 (10⁻⁴ dilution)=>11 particles in 100 μl (100 μl of 10⁻⁴ dilution was added to 100 μl of HEK-293 cells for AdenoX)=>11×10⁴ particles in 100 μl of original sample=>11×10⁵ pfu/ml
69 (10⁻³ dilution)=>69 particles in 100 μl (100 μl of 10⁻³ dilution was added to 100 μl of HEK-293 cells for AdenoX)=>69×10³ particles in 100 μl of original sample=>69×10⁴ pfu/ml
Mean GEN1 titre=>2.26×10⁶ pfu/ml

The mean titre for GEN3 was therefore approximately 40-fold higher than for GEN1.

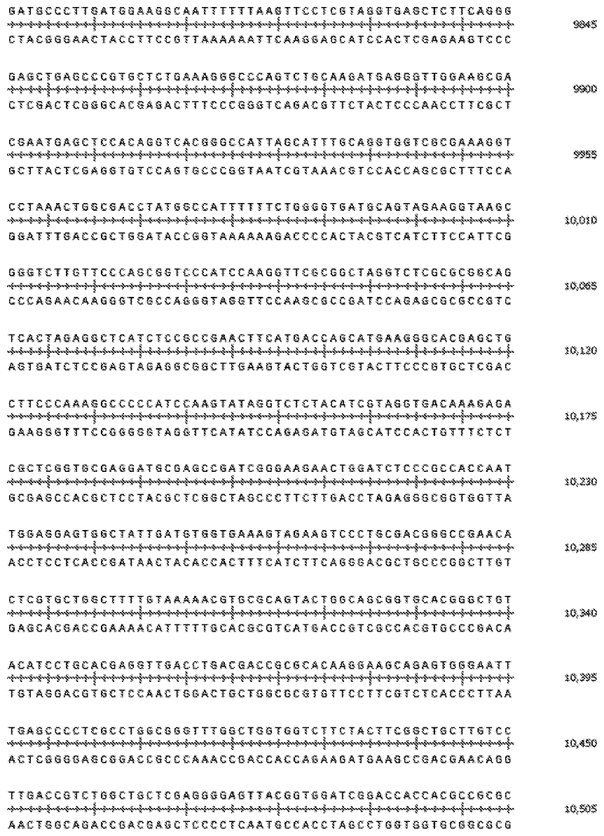

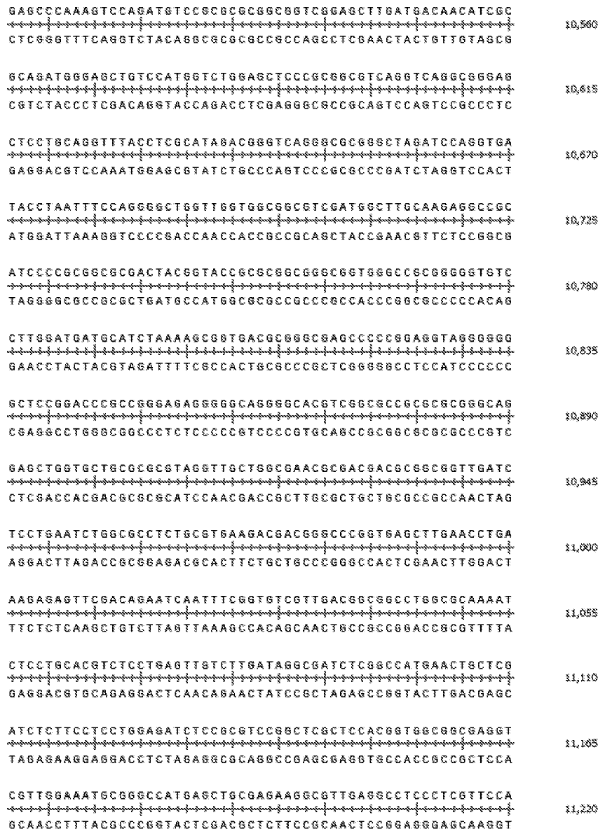

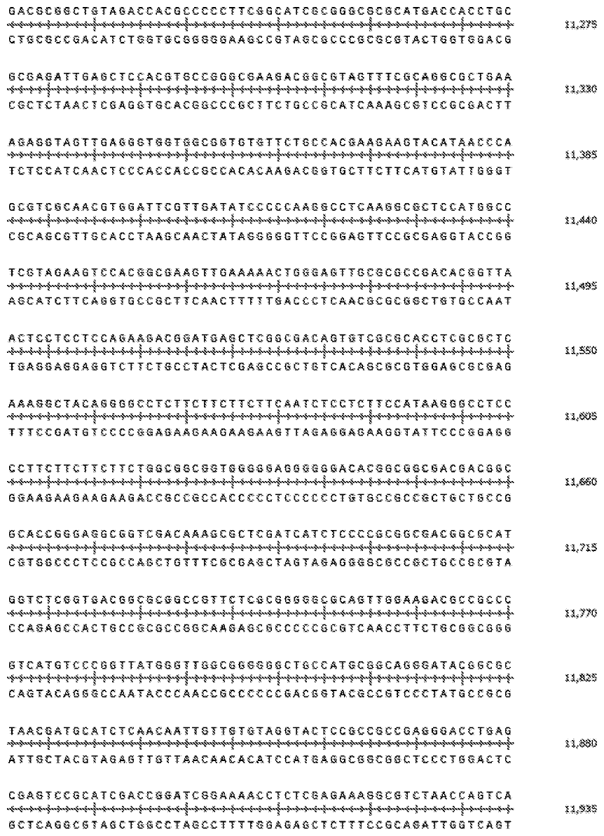

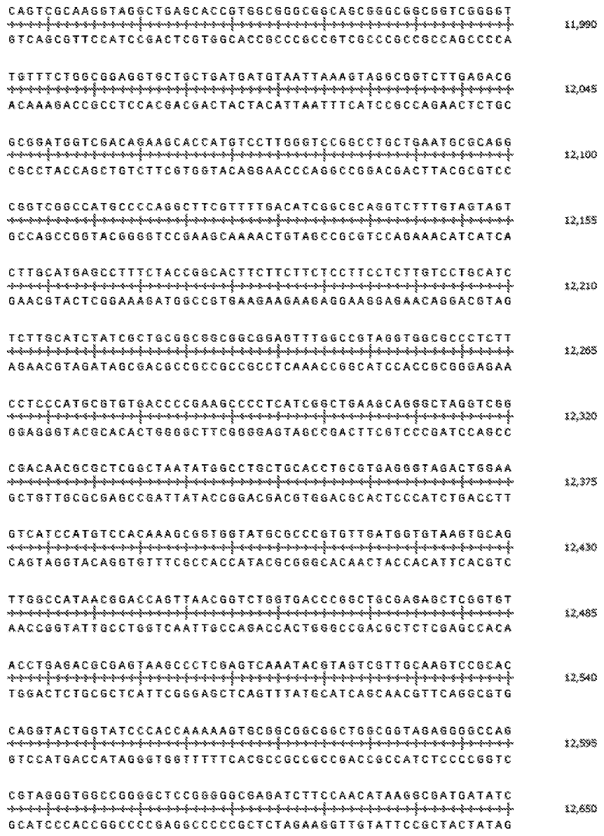

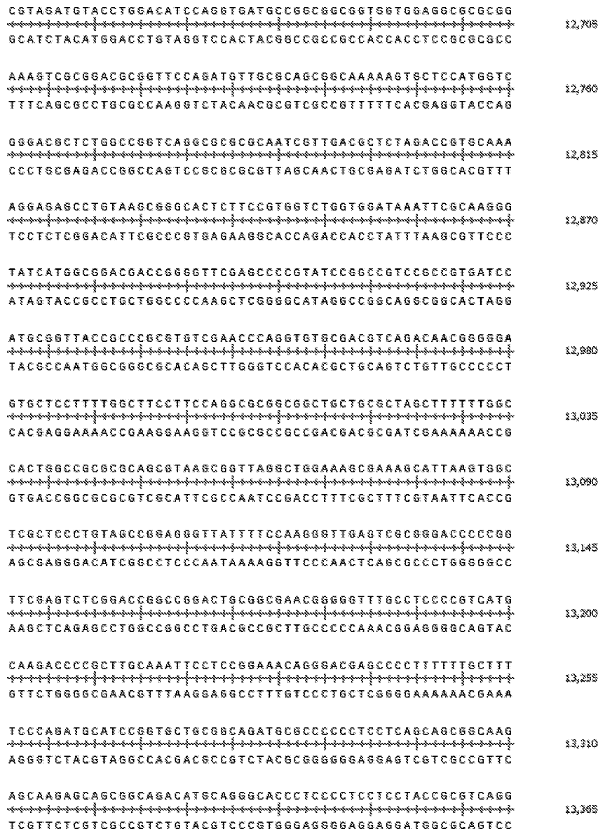

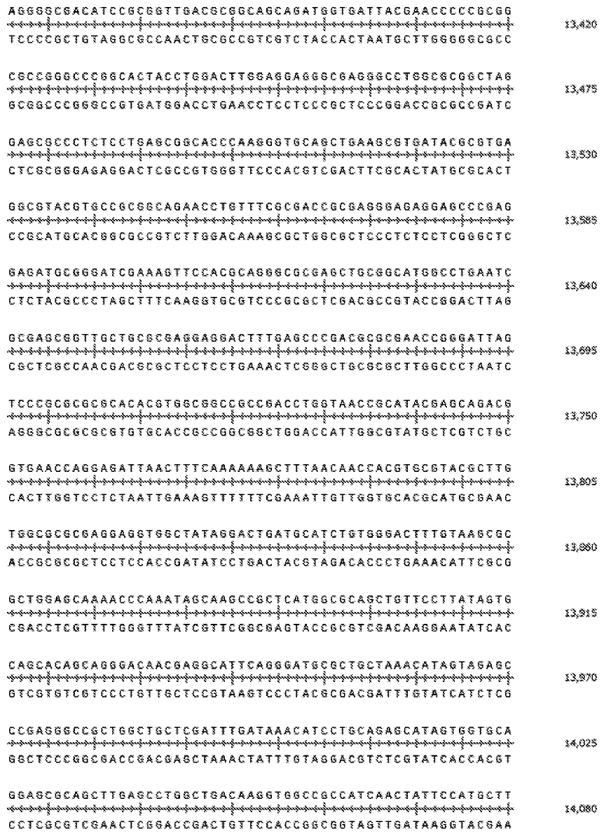

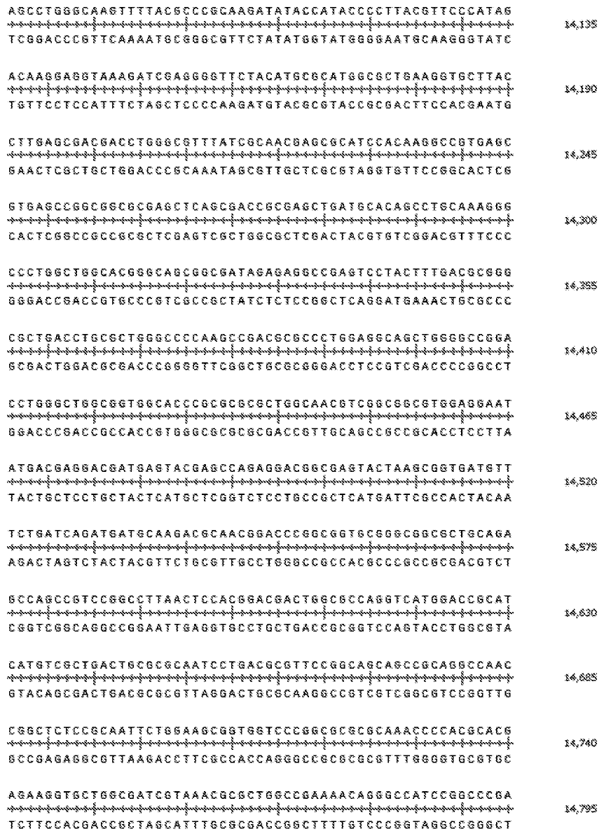

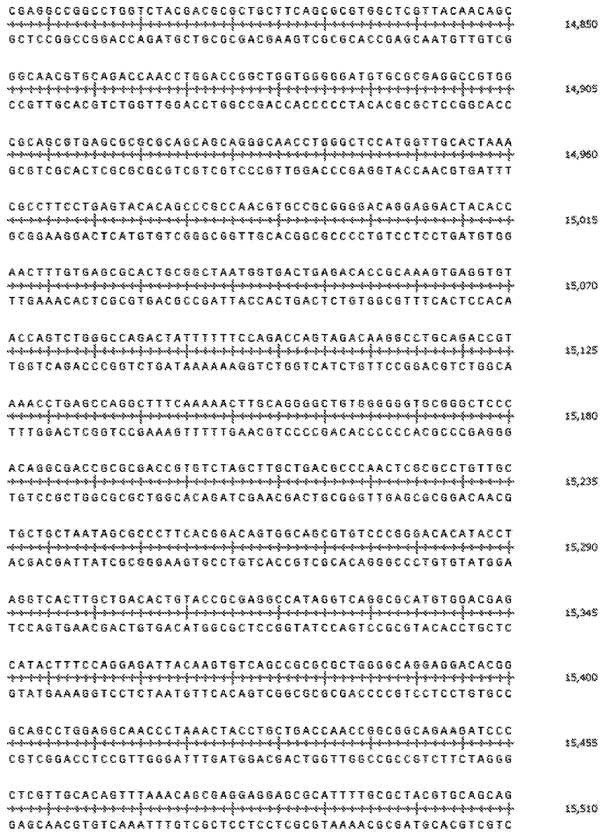

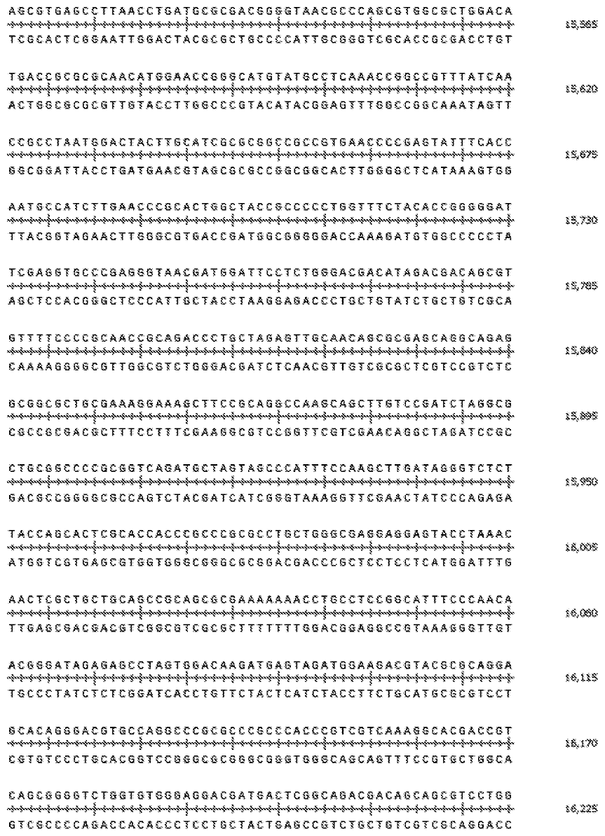

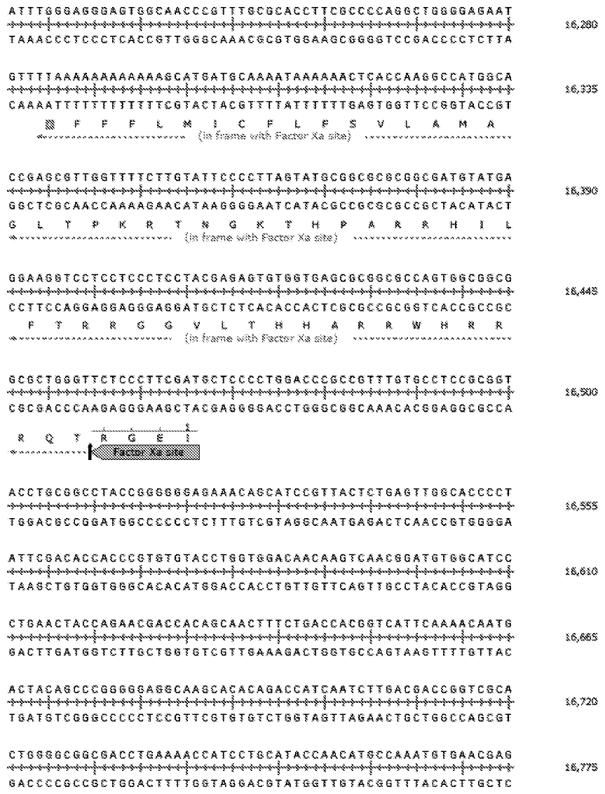

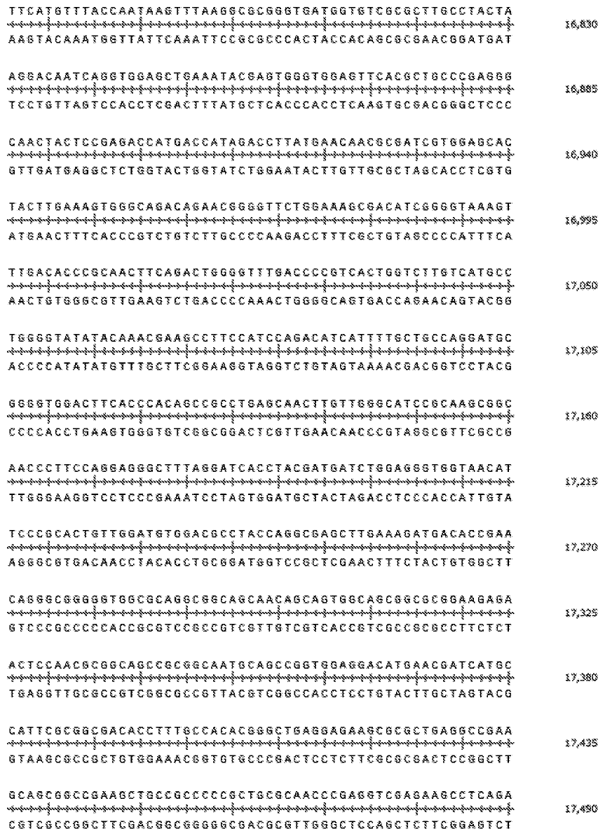

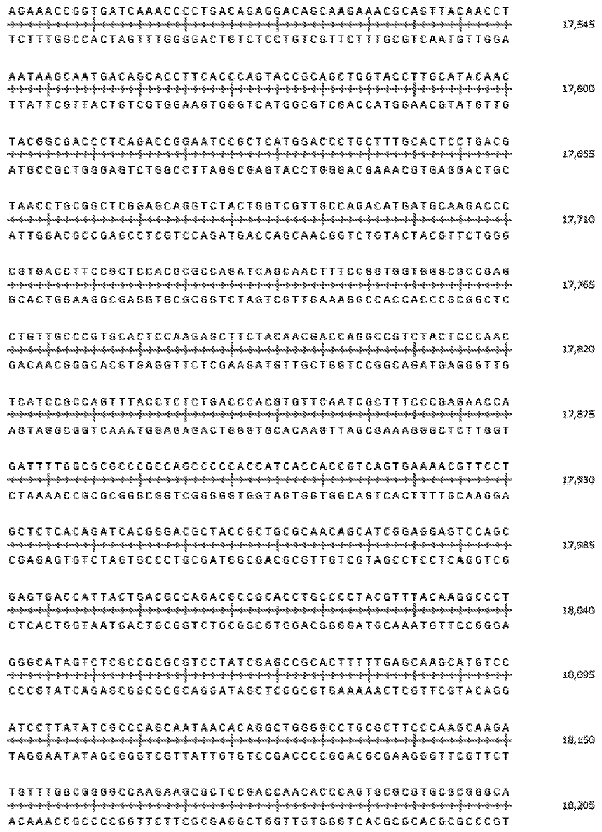

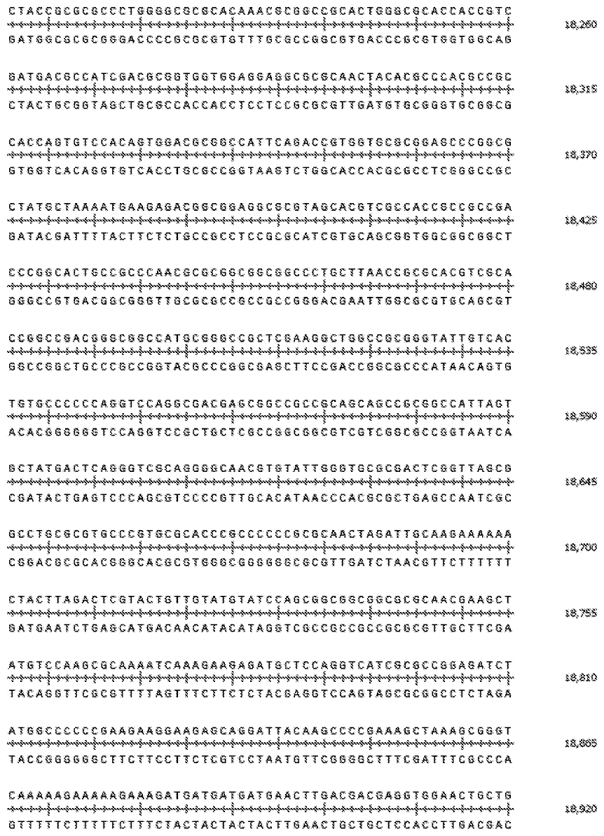

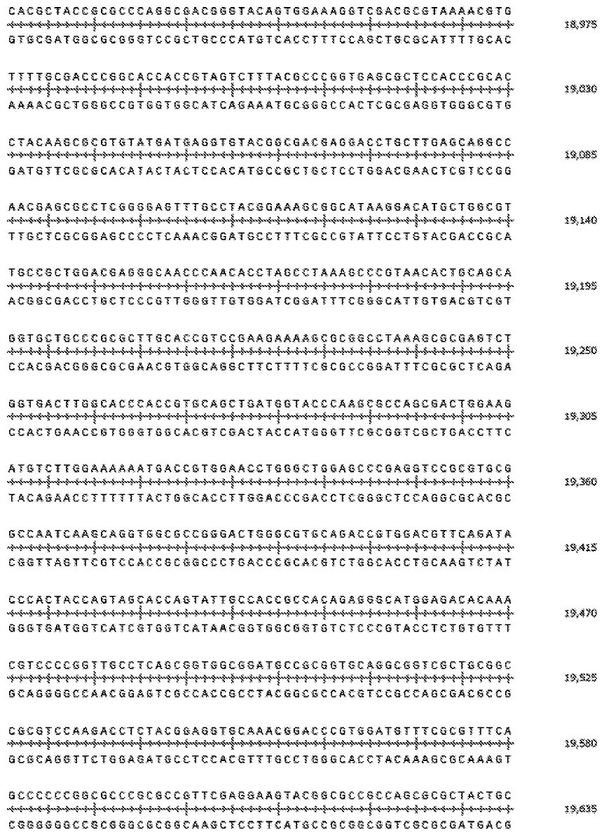

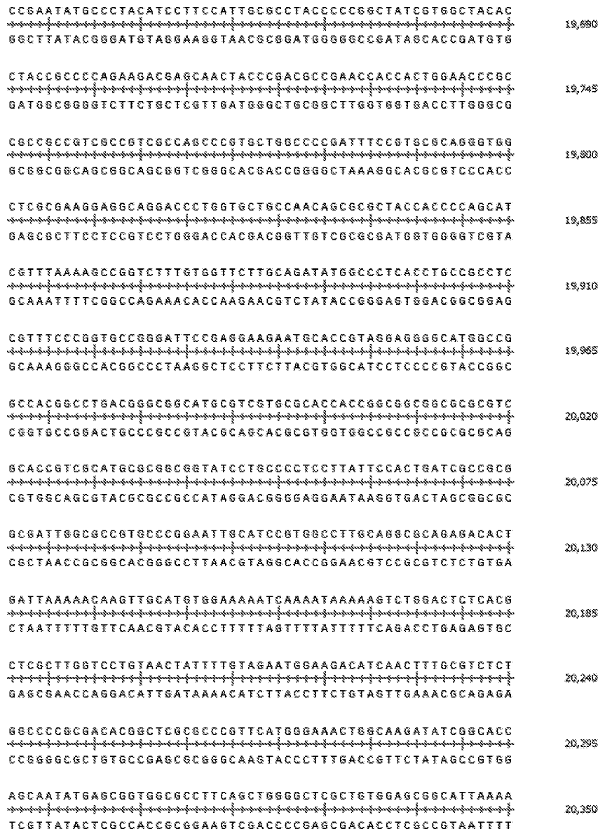

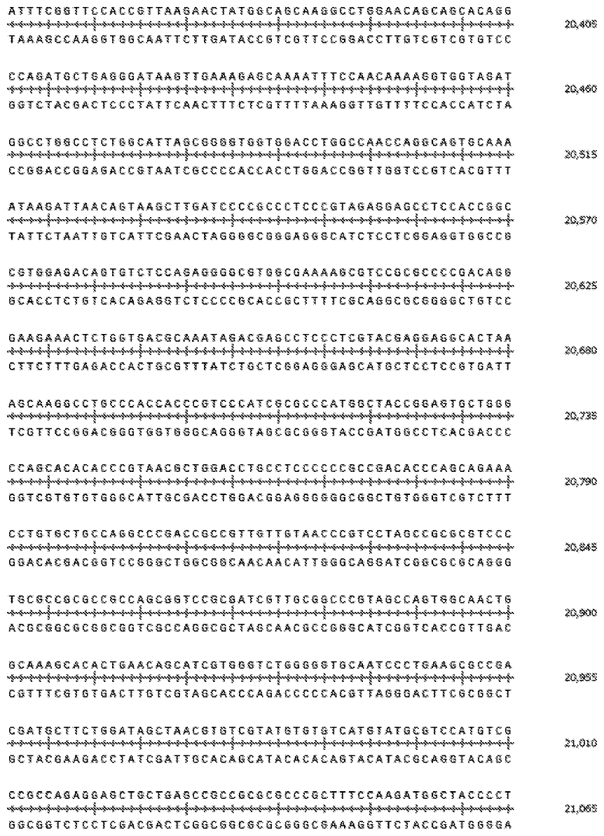

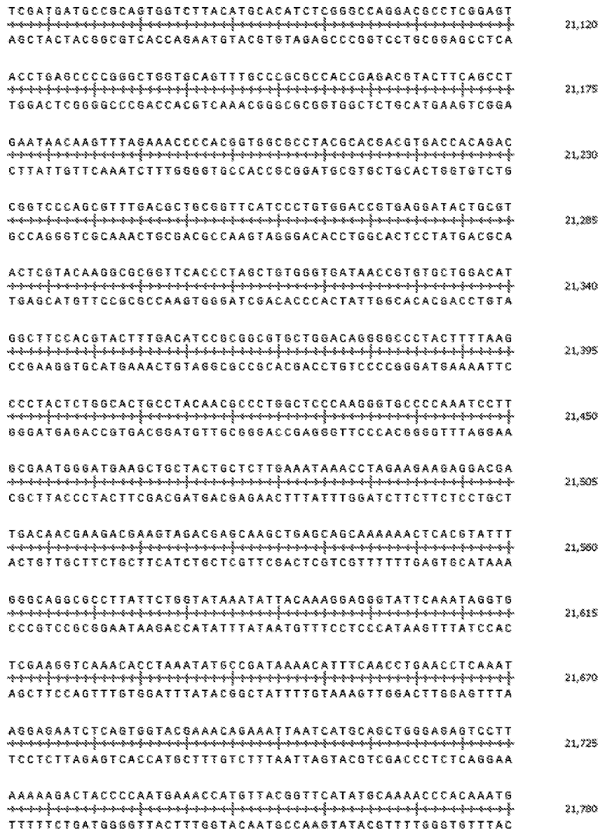

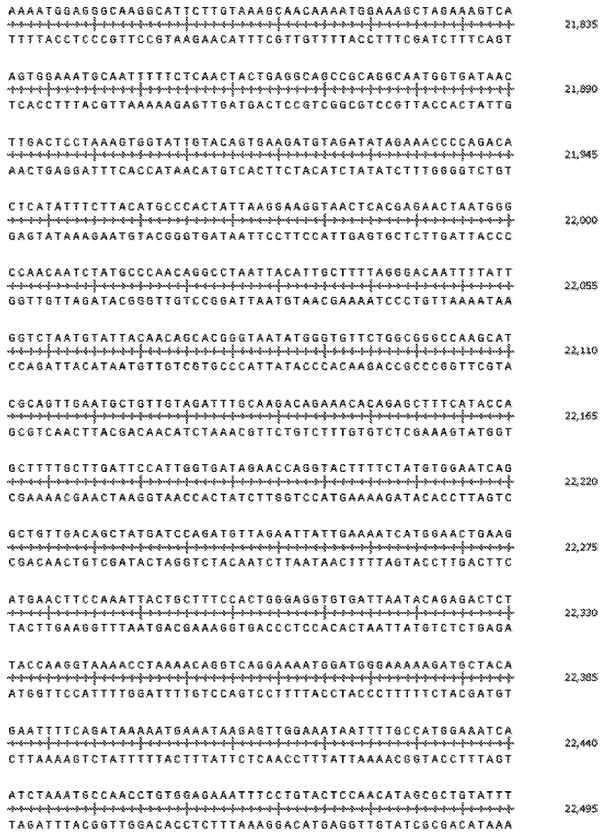

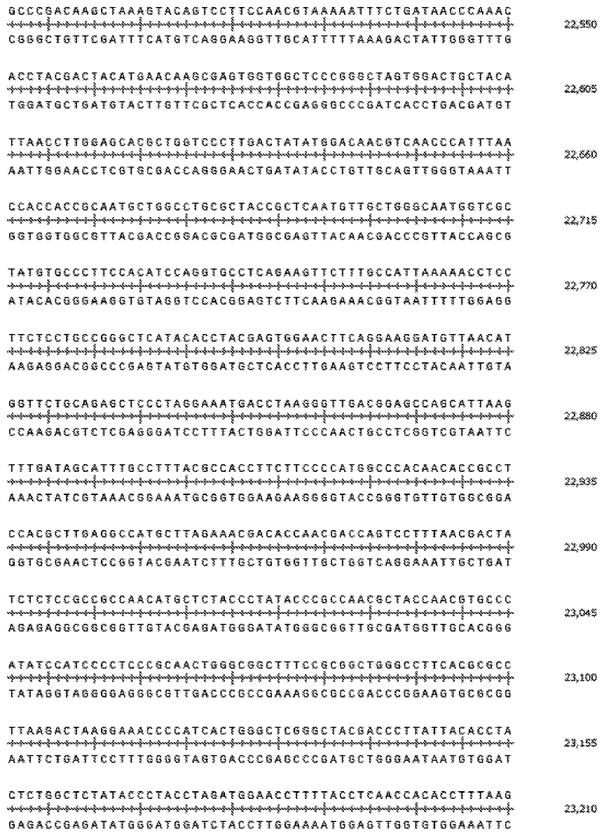

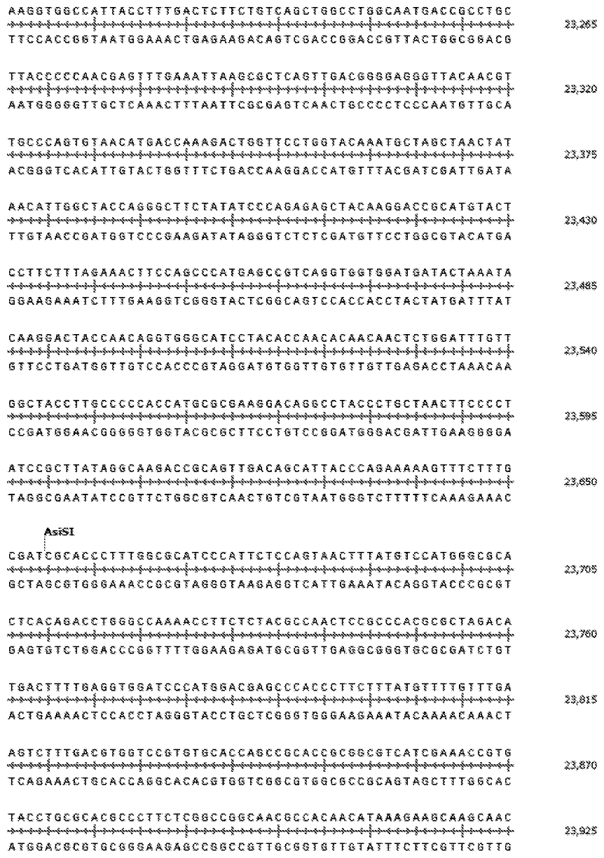

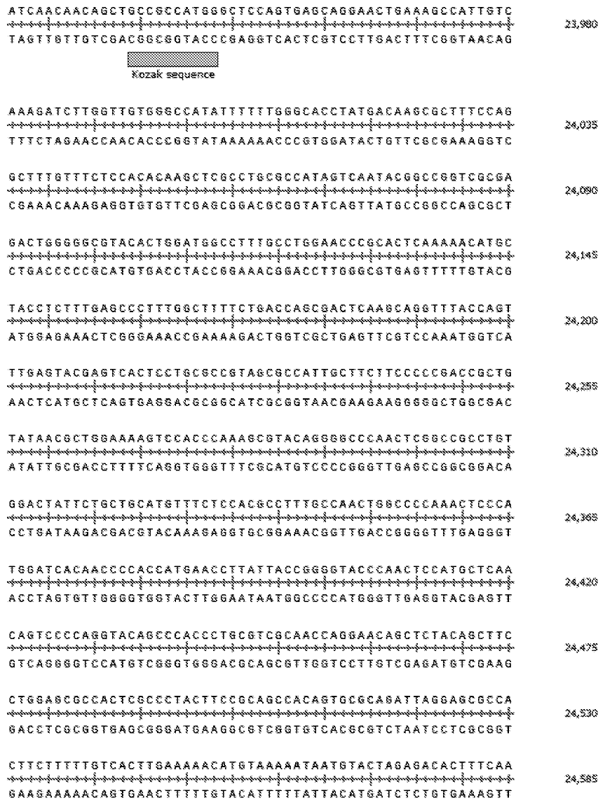

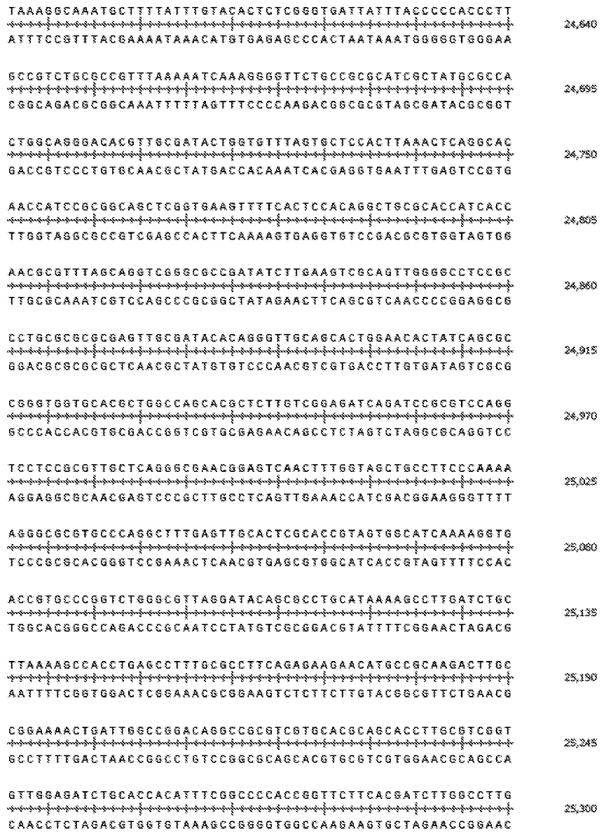

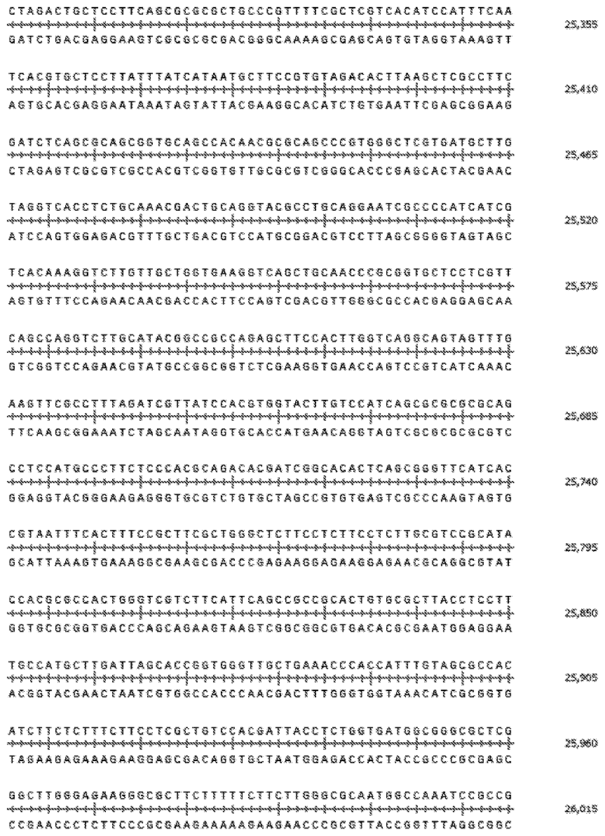

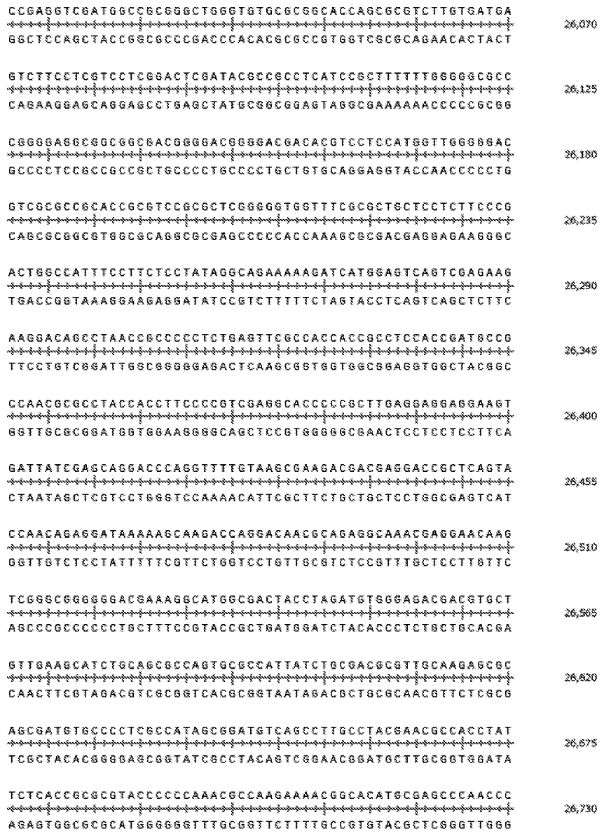

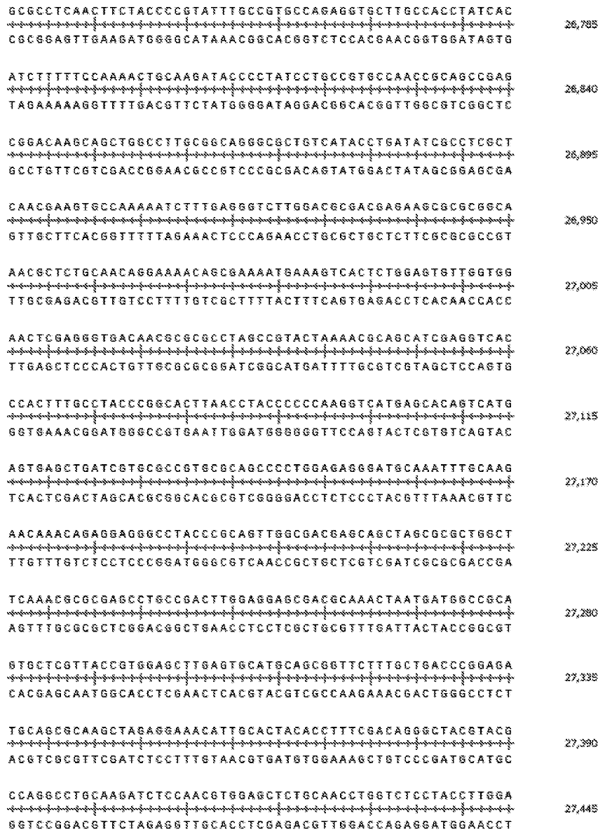

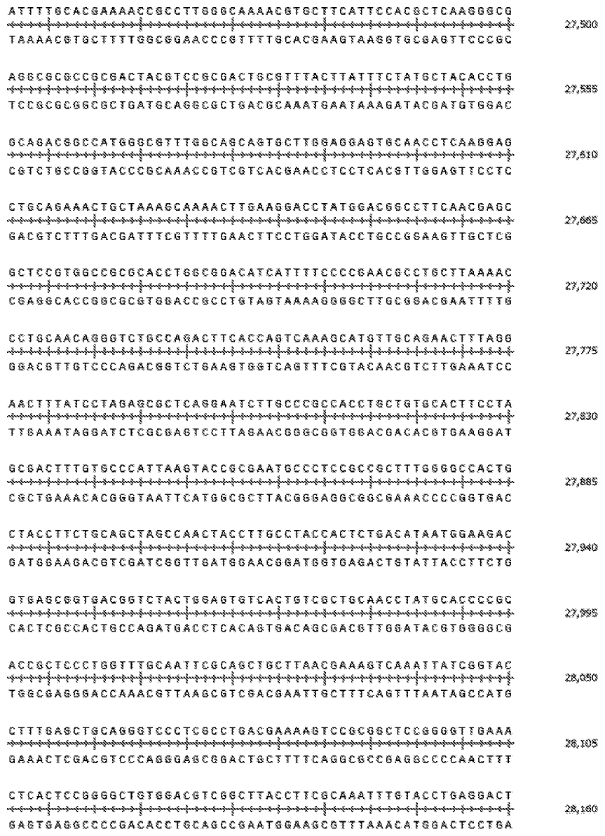

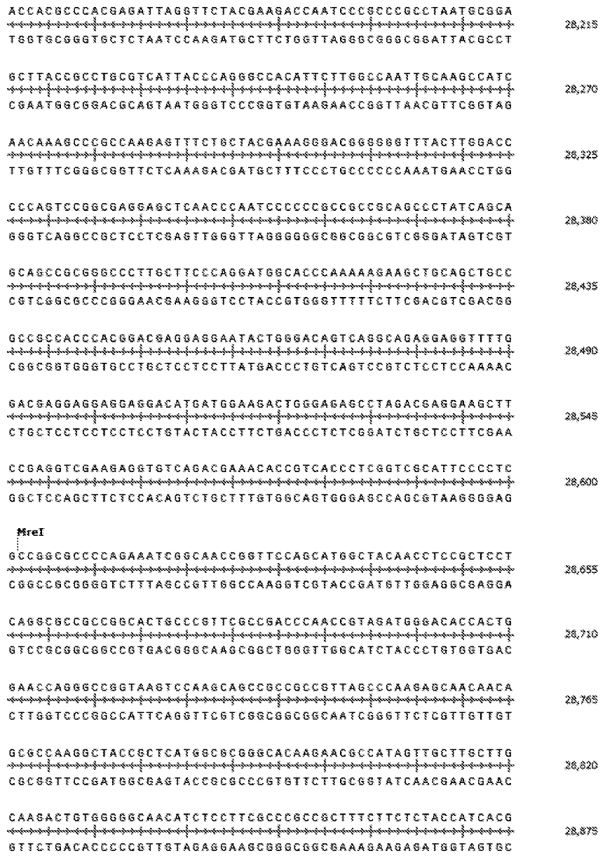

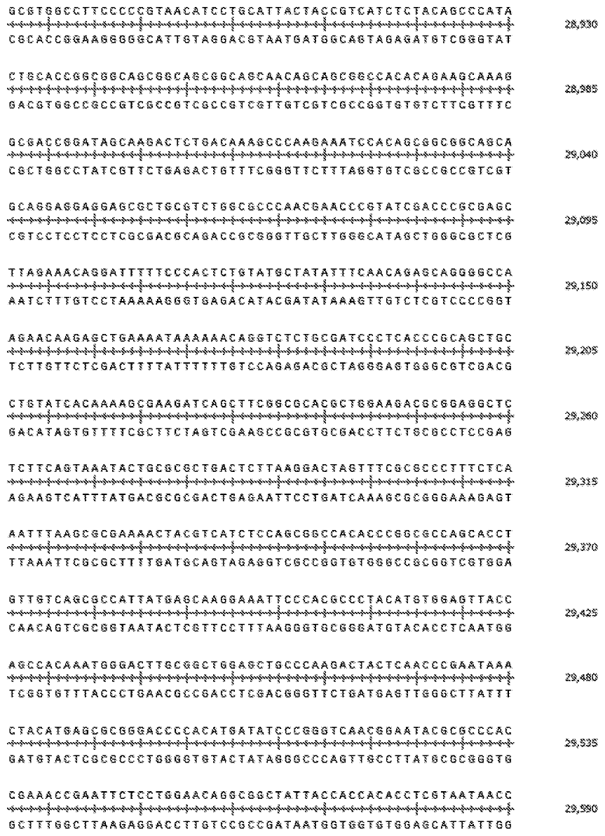

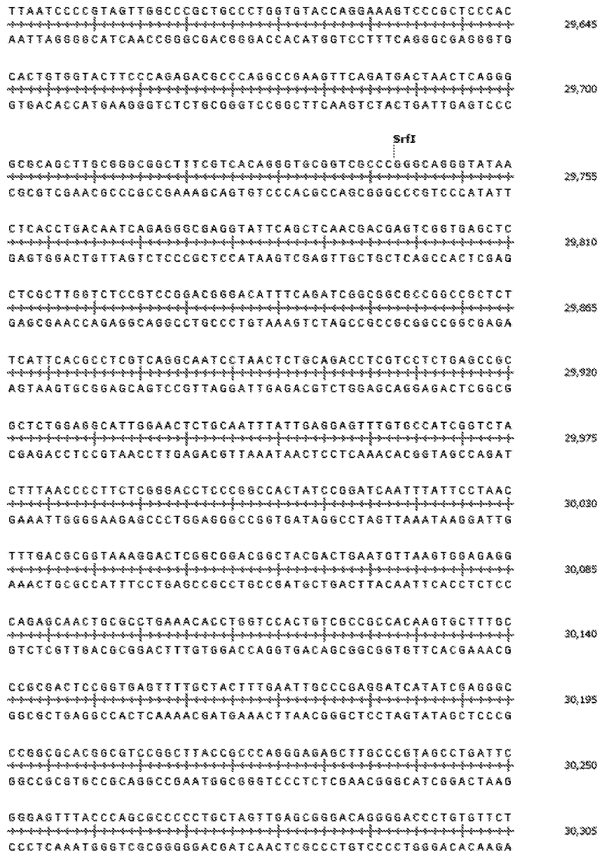

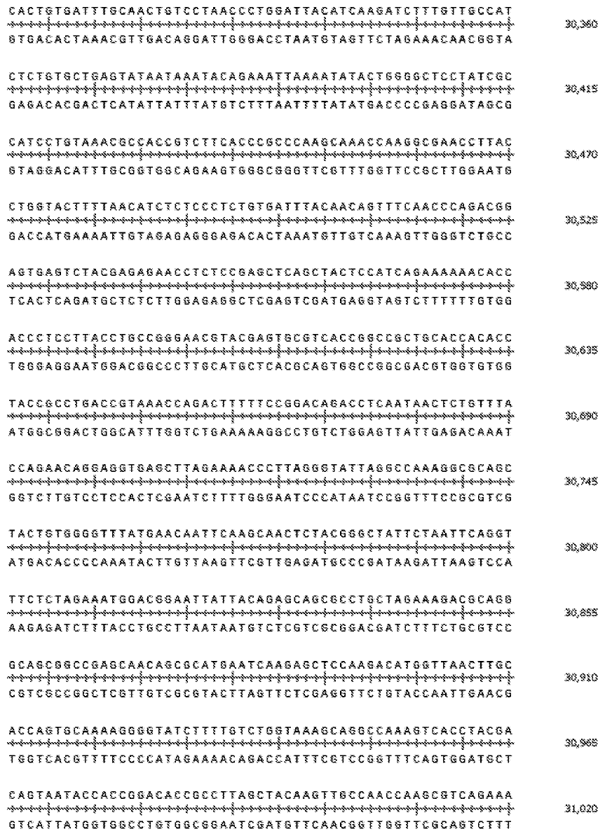

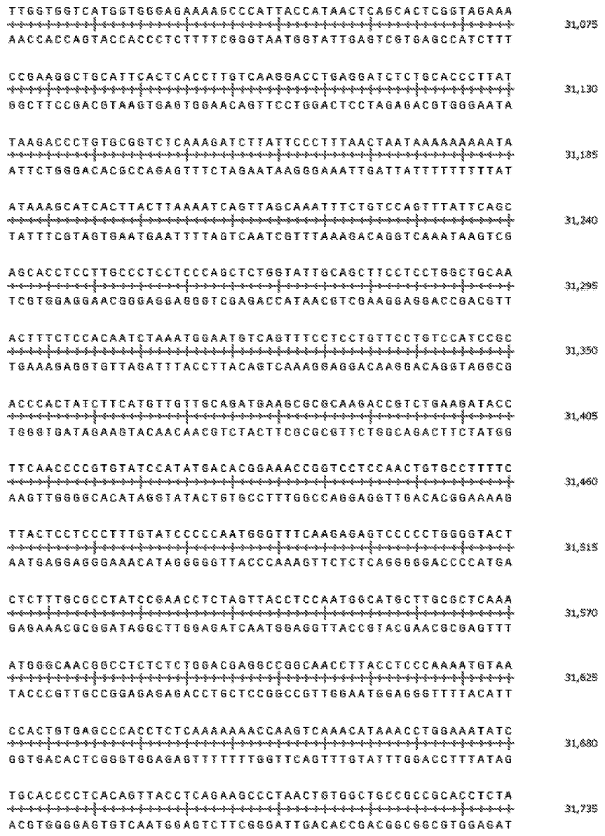

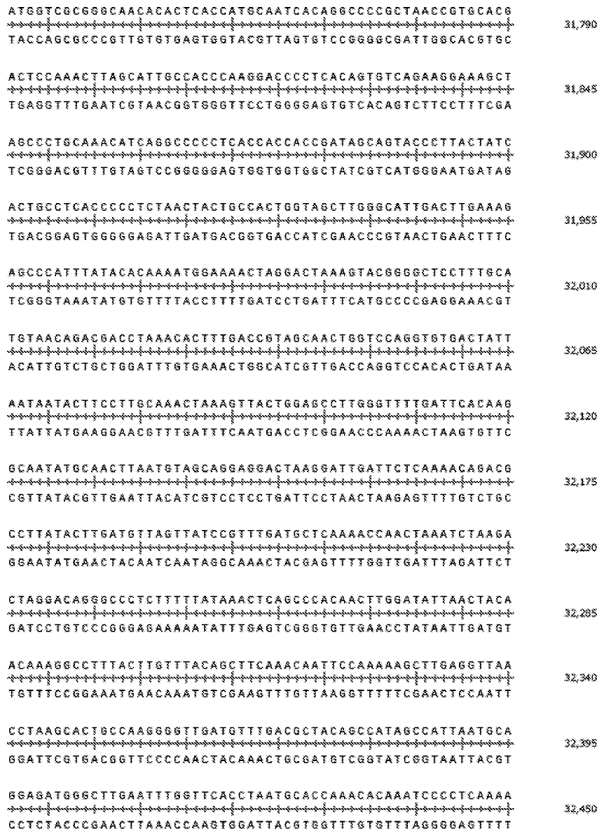

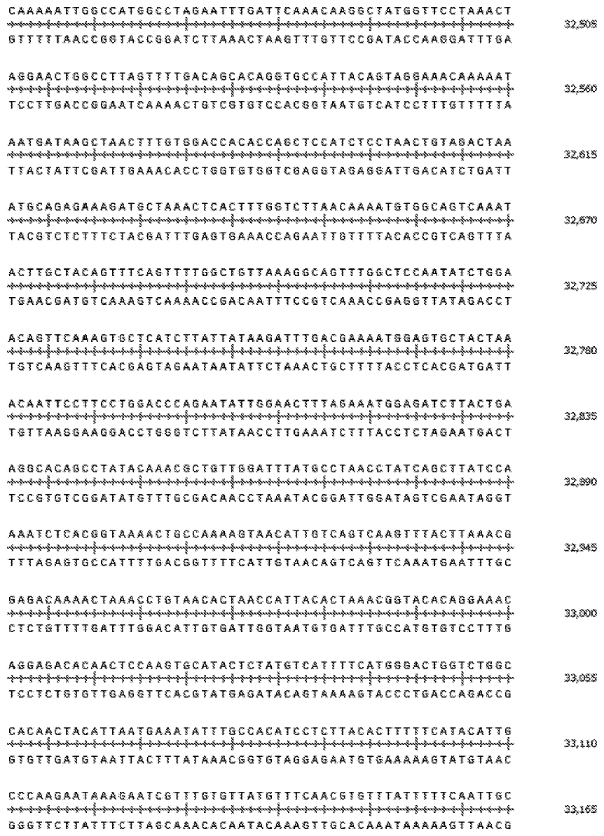

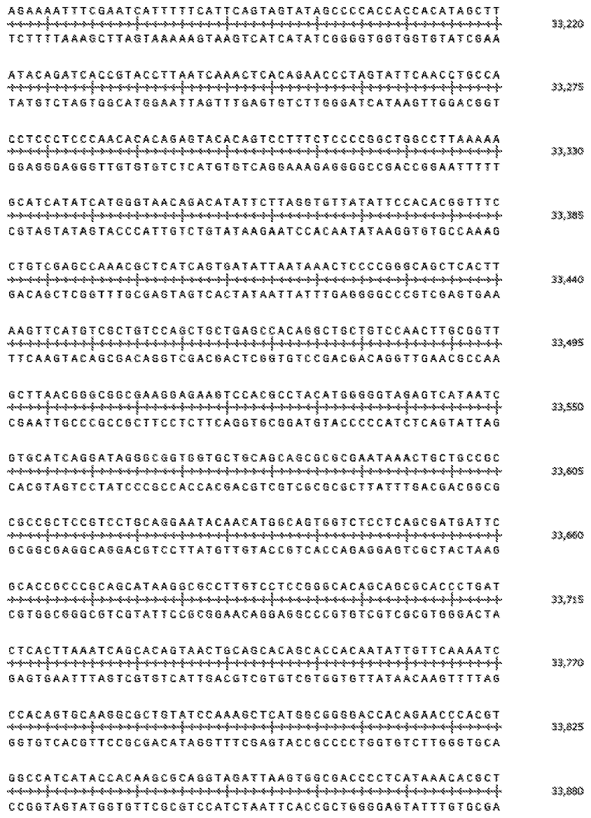

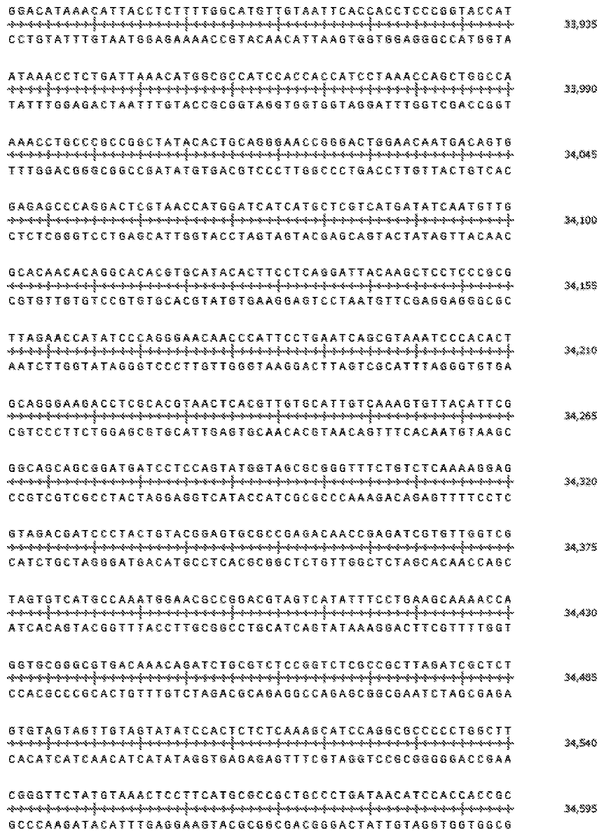

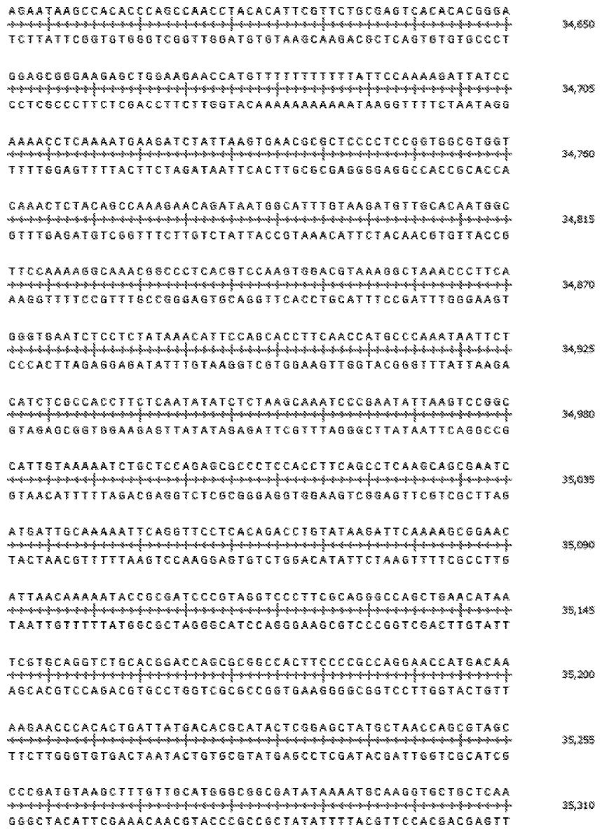

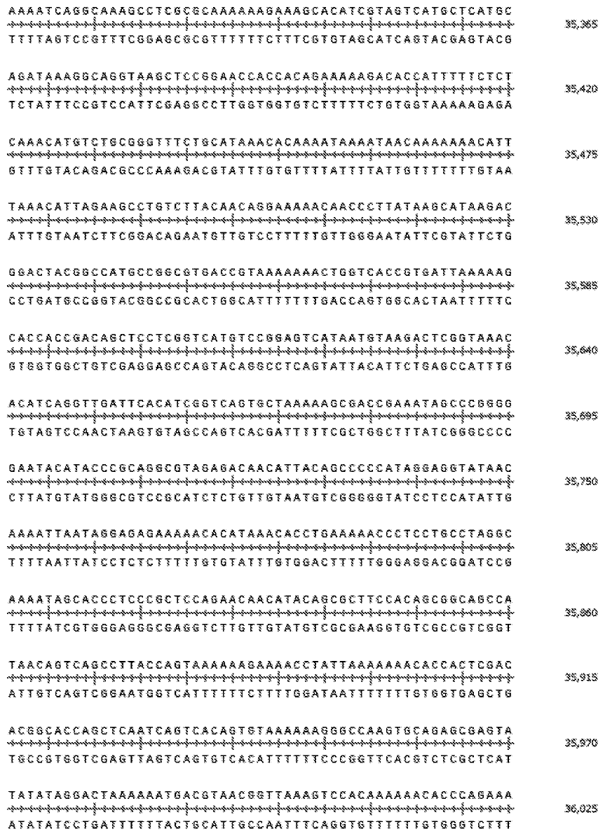

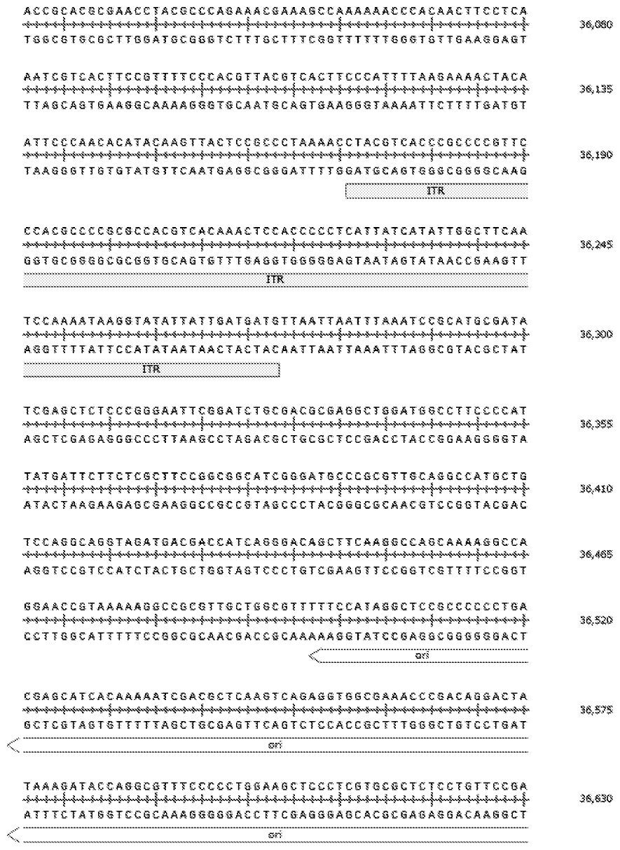

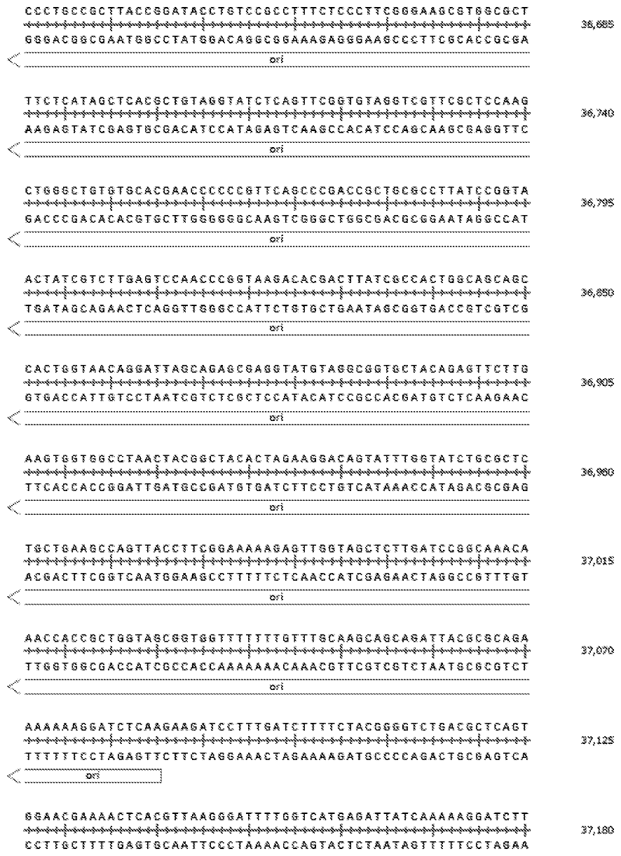

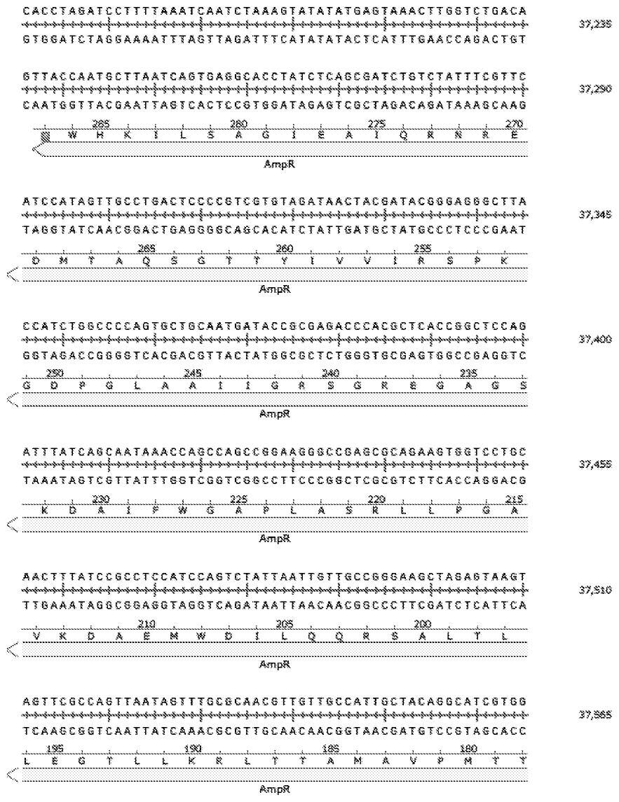

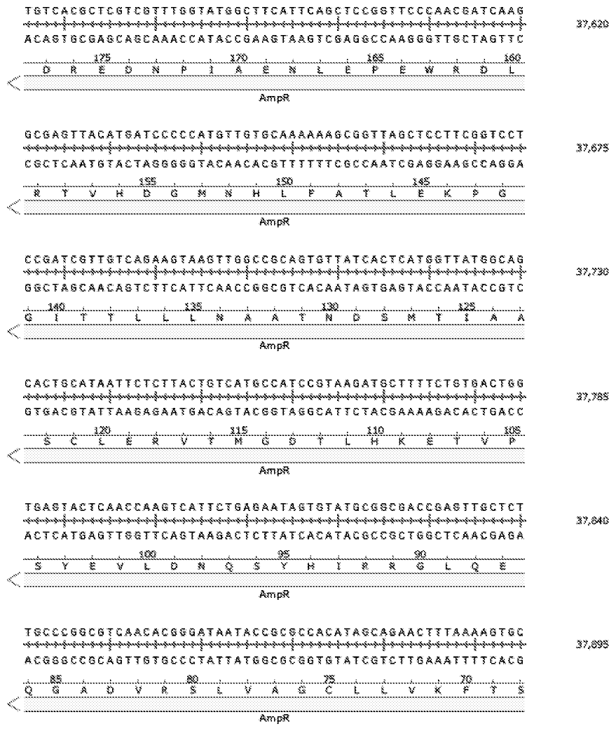

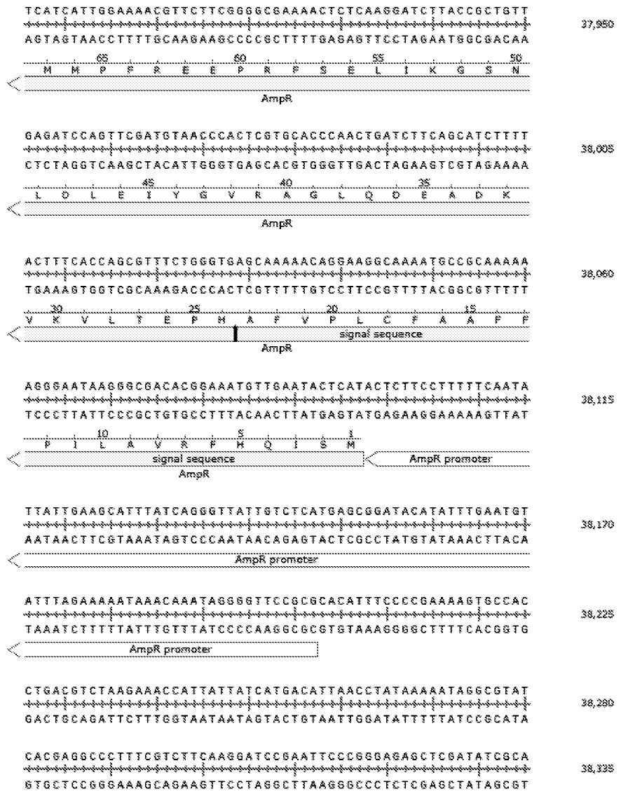

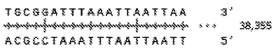

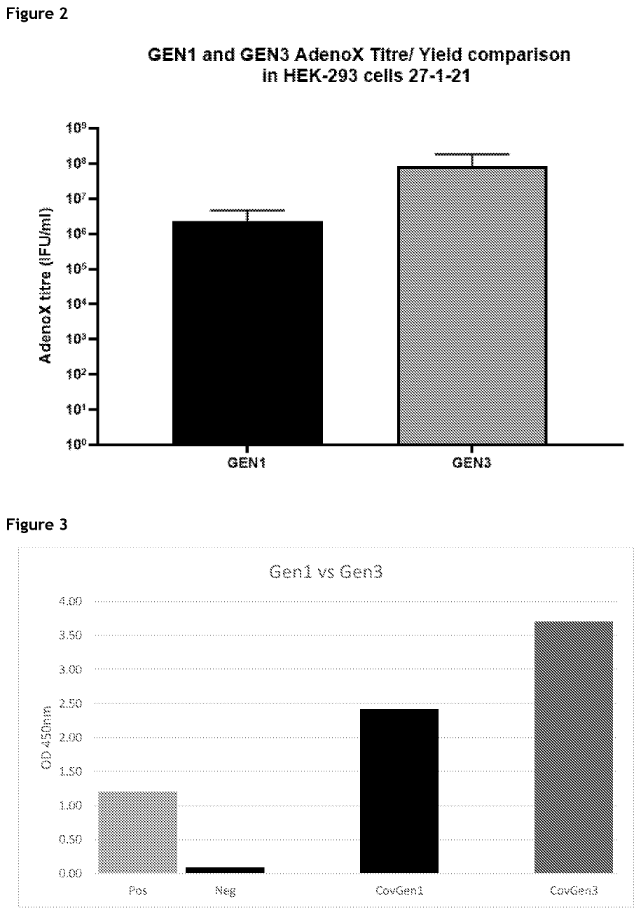

FIG. 2 presents a comparison of the GEN1 and GEN3 yields.

### Example 2—Immunological Responses

Immunological responses were investigated following administration of the GEN1 and GEN3 viruses to rats using the EUROIMMUN anti-SARS-CoV2 ELISA kit. The EUROIMMUN anti-SARS-CoV2 IgG ELISA kit is designed to provide semiquantitative in vitro determination of human antibodies (IgG) against SARS-CoV-2 in serum or plasma. Deviations from the kit protocol were though made in order to detect rat IgG antibodies as described below:

Rat plasma samples were diluted 1:10 (11 μl in 99 μl Euroimmun kit buffer). Samples were mixed by vortexing. 100 μl of diluted sample was added to the relevant well on a Euroimmun Sars Cov-2 IgG ELISA plate. Plates were covered and incubated for 1.5 h at 37° C. Plates were washed 3× with 300 μl Euroimmun kit wash buffer (1×). Plates were banged dry. Anti-Rat IgG (whole molecule) biotin (SIGMA, B7139, SLBM7228V) was diluted 1:20000 in PBS and added at 100 μl/well. Plates were covered and incubated for 1 h at room temperature. Plates were washed 3× with 300 μl Euroimmun kit wash buffer (1×). Plated were banged dry, 100 μl/well streptavidin-HRP reagent 1× was added (VIRUSYS, SA-HRP, LOT #826503, 09/2020). Plates were covered and incubated for 30 minutes at room temperature. Plates were washed 3× with 300 μl Euroimmun kit wash buffer (1×). Plates were banged dry. 100 μl/well Euroimmun kit substrate solution was added. Plates were covered and incubated for 15 minutes at room temperature in the dark. 100 μl/well Euroimmun kit stop solution was added and plates were read at 450 nm.

Results are presented in FIG. 3, which shows higher levels of IgG for the GEN3 antigen, compared with GEN1.

1. A polynucleotide encoding an antigen from a virus, wherein the antigen has been modified (i) to target the antigen to the plasma membrane and/or (ii) to reduce membrane fusion and/or the formation of syncytia.

2. The polynucleotide of claim 1, wherein the antigen is a Class I, Class II or Class III membrane fusion protein.

3. The polynucleotide of claim 1 or 2, wherein targeting to the plasma membrane is increased by modifying the cytoplasmic tail of the antigen, such as wherein a polybasic motif is substituted, optionally a dibasic motif.

4. The polynucleotide of claim 3, wherein the polybasic motif is substituted with alanine.

5. The polynucleotide of any one of the preceding claims, wherein the cytoplasmic tail is replaced with the cytoplasmic tail from infectious bronchitis virus in which the dilysine motif is substituted, such as wherein the dilysine motif is substituted with alanine, optionally wherein:
(a) the tyrosine internalisation sequence is also mutated to replace the tyrosines, optionally with alanine; and/or
(b) the cytoplasmic tail comprises one or more additional amino acid substitutions, deletions or insertions.

6. The polynucleotide of claim 5, wherein the cytoplasmic tail of the modified antigen comprises the following sequence:
(SEQ ID NO: 7)
GCCGCCCGCFGIIPLMSKCGKKSSAATTFDNDVVTEQYRPAASV.

7. The polynucleotide of any one of the preceding claims, wherein the modifications to reduce membrane fusion and/or the formation of syncytia comprise the substitution of tryptophan residues, optionally wherein the tryptophan residues are in a transmembrane domain of the antigen, such as in an aromatic stretch of the transmembrane domain.

8. The polynucleotide of claim 7, wherein the tryptophan residues are substituted with alanine.

9. The polynucleotide of claim 8, wherein the motif WXWXXW (SEQ ID NO: 11) is replaced with AXAXXA (SEQ ID NO: 12).

10. The polynucleotide of any one of the preceding claims, wherein the antigen is from a coronavirus, such as wherein the antigen is the spike (S) protein of SARS-CoV-2.

11. The polynucleotide of claim 10, which encodes a SARS-CoV-2 spike protein comprising the sequence KTYIKAPAYVALAIAFATIIFILILGWLFFMTGCCGCCCGCFGIIPLMSKCGK KSSAATTFDNDVVTEQYRPAASV (SEQ ID NO: 16) or YIKAPAYVALAIAFATIIFILILGWLFFMTGCCGCCCGCFGIIPLMSKCGKKS SAATTFDNDVVTEQYRPAASV (SEQ ID NO: 42), or comprising:
(a) a variant with at least 90% identity to the above sequences; or
(b) a variant of the above sequences with up to 10 deletions, substitutions or additions.

12. The polynucleotide of claim 11, wherein the variant retains the AXAXXA (SEQ ID NO: 12) motif and the AA residues at the −3 and −4 positions.

13. The polynucleotide of any one of the preceding claims, which encodes a SARS-CoV-2 spike protein comprising: (a) the following sequence, or encodes a spike protein with at least 90% identity to the following sequence FVFLVLLPLVSSQCVNLTTRTQLPPAYTNSFTRGVYYPDKVFRSSVLHSTQD LFLPFFSNVTWFHAIHVSGTNGTKRFDNPVLPFNDGVYFASTEKSNIIRGWI FGTTLDSKTQSLLIVNNATNVVIKVCEFQFCNDPFLGVYYHKNNKSWMESE FRVYSSANNCTFEYVSQPFLMDLEGKQGNFKNLREFVFKNIDGYFKIYSKH TPINLVRDLPQGFSALEPLVDLPIGINITRFQTLLALHRSYLTPGDSSSGWTA GAAAYYVGYLQPRTFLLKYNENGTITDAVDCALDPLSETKCTLKSFTVEKG IYQTSNFRVQPTESIVRFPNITNLCPFGEVFNATRFASVYAWNRKRISNCVA DYSVLYNSASFSTFKCYGVSPTKLNDLCFTNVYADSFVIRGDEVRQIAPGQ TGKIADYNYKLPDDFTGCVIAWNSNNLDSKVGGNYNYLYRLFRKSNLKPF ERDISTEIYQAGSTPCNGVEGENCYFPLQSYGFQPTNGVGYQPYRVVVLSFE LLHAPATVCGPKKSTNLVKNKCVNFNFNGLTGTGVLTESNKKFLPFQQFG RDIADTTDAVRDPQTLEILDITPCSFGGVSVITPGTNTSNQVAVLYQDVNCT EVPVAIHADQLTPTWRVYSTGSNVFQTRAGCLIGAEHVNNSYECDIPIGAGI CASYQTQTNSPRRARSVASQSIIAYTMSLGAENSVAYSNNSIAIPTNFTISVT TEILPVSMTKTSVDCTMYICGDSTECSNLLLQYGSFCTQLNRALTGIAVEQD KNTQEVFAQVKQIYKTPPIKDFGGFNFSQILPDPSKPSKRSFIEDLLFNKVTL ADAGFIKQYGDCLGDIAARDLICAQKFNGLTVLPPLLTDEMIAQYTSALLA GTITSGWTFGAGAALQIPFAMQMAYRENGIGVTQNVLYENQKLIANQFNS AIGKIQDSLSSTASALGKLQDVVNQNAQALNTLVKQLSSNFGAISSVLNDIL SRLDKVEAEVQIDRLITGRLQSLQTYVTQQLIRAAEIRASANLAATKMSECV LGQSKRVDFCGKGYHLMSFPQSAPHGVVFLHVTYVPAQEKNFTTAPAICH DGKAHFPREGVFVSNGTHWFVTQRNFYEPQIITTDNTFVSGNCDVVIGIVN NTVYDPLQPELDSFKEELDKYFKNHTSPDVDLGDISGINASVVNIQKEIDRL NEVAKNLNESLIDLQELGKYKTYIKAPAYVALAIAFLTIIFILILGWVFFMTG CCGCCCGCFGIIPLMSKCSKKSSAATTFDNDVVTEQYRPAASV (SEQ ID NO: 17), or (b) the sequence as shown in FIG. 1 or with at least 90% identity to the sequence shown in FIG. 1.

14. A viral vector comprising the polynucleotide of any one of claims 1-13.

15. The viral vector of claim 14, which is an adenoviral vector.

16. The viral vector of claim 15 , which is a type 5 adenoviral vector.

17. The viral vector of claim 16, which is a ΔE1, ΔE3 vector.

18. The viral vector of any one of claims 14-17, wherein the polynucleotide encoding the modified antigen is under control of a CMV promoter.

19. The viral vector of any one of claims 14-18, which encodes a modified SARS-CoV-2 spike protein and which additionally encodes a further SARS-CoV-2 antigen, such as the SARS-CoV-2 nucleocapsid.

20. A polypeptide encoded by the polynucleotide of any one of claims 1-13.

21. Viral particles carrying the polynucleotide of any one of claims 1-13.

22. Viral particles of claim 21, which comprise a vector of any one of claims 14-19.

23. The viral particles of claim 22, wherein the virus is an adenovirus.

24. The viral particles of claim 23, wherein the virus is a type 5 adenovirus.

25. The viral particles of claim 23 or 24, wherein the virus is replication incompetent, such as a ΔE1, ΔE3 virus.

26. A vaccine comprising the viral particles of any one of claims 21-25.

27. The vaccine of claim 26, which is for oral administration to a subject in need thereof.

28. A method of producing viral particles of claim 21, said method comprising culturing host cells comprising a vector of any one of claims 14-19 under conditions permitting production of the virus and harvesting the virus produced by the host cells.

29. The method of claim 28, wherein the viral particles are adenoviral particles, such as adenoviral particles as defined in any one of claims 23-25.

30. The method of claim 28 or 29, wherein the method further comprises expanding and/or purifying the virus particles.

31. The method of claim 28, 29 or 30, wherein the method further comprises formulating the viral particles for administration to a subject in need thereof.

32. The method of any one of claims 28-31, wherein the host cells are HEK293 cells.
